(19) United States
(12) Patent Application Publication
(10) Pub. No.: US 2022/0214282 A1
(43) Pub. Date: Jul. 7, 2022

(54) SENSORS AND METHODS USING ELECTROCHEMILUMINESCENCE OF METAL NANOCLUSTERS

(71) Applicant: Georgia State University Research Foundation, Inc., Atlanta, GA (US)

(72) Inventors: Gangli Wang, Atlanta, GA (US); Shuang Chen, Atlanta, GA (US); Tanyu Wang, Atlanta, GA (US)

(21) Appl. No.: 17/610,892

(22) PCT Filed: May 15, 2020
(86) PCT No.: PCT/US2020/033128

§ 371 Date: Nov. 12, 2021

Related U.S. Application Data
(60) Provisional application No. 62/848,152, filed on May 15, 2019.
(60) Provisional application No. 62/853,549, filed on May 28, 2019.
(60) Provisional application No. 62/854,668, filed on May 30, 2019.

(51) Int. Cl. G01N 21/66; G01N 21/76; G01N 33/487

(52) U.S. Cl. CPC G01N 21/66; G01N 2201/067; G01N 33/487; G01N 21/76

(57) ABSTRACT

Disclosed are sensors and methods using electrochemiluminescence (ECL) of metal nanoclusters. The ECL sensors containing metal nanoclusters disclosed herein have high signal output and high signal/noise ratio. Highly effective sensing methods using these ECL sensors that is rapid, simple, and allows for sensitive and specific detection of analytes of interest at a low cost are also disclosed.

### CROSS-REFERENCE TO RELATED APPLICATIONS

This application claims the benefit of and priority to U.S. Provisional Application No. 62/848,152 filed May 15, 2019, U.S. Provisional Application No. 62/853,549 filed May 28, 2019, and U.S. Provisional Application No. 62/854,668 filed May 30, 2019, which are hereby incorporated by reference in their entirety.

### FIELD OF THE INVENTION

The invention is generally in the field of sensors and methods using electrochemiluminescence, more particularly to sensors and methods using electrochemiluminescence of metal nanoclusters.

### BACKGROUND OF THE INVENTION

Electrogenerated chemiluminescence or electrochemiluminescence (ECL) has attracted significant research interests for decades. Fundamental research spans from the synthetic efforts on developing new ECL reagents to physical and analytical studies on establishing better reaction pathways and analysis strategies (Miao, Chem. Rev., 108(7):2506-2553 (2008); Gross, et al., Bioanalysis, 8(19):2071-2089 (2016); Hesari, et al., Acc. Chem. Res., 50(2):218-230 (2017)). ECL is a subclass of chemiluminescence (CL), in which electroactive materials are oxidized or reduced at an electrode under appropriate potentials to form active species that relax to ground state and emit light (Richter, Chem. Rev., 104(6):3003-3036 (2004); Bertoncello, et al., Chem Electro Chem, 4(7):1663-1676 (2017)). ECL is often favored over homogeneous CL because of the active control and localized signal generation at the electrode-solution interface, a much more versatile detection platform (Tan, et al., Angew. Chem. Int. Ed., 53(37):9822-9826 (2014)). An essential demand for basic research is to achieve high contrast, mostly focused on improving the relatively low signal, driven by the widespread applications for ECL in sensors and assays because of the greatly simplified ECL instrumentation (Miao, Chem. Rev., 108(7):2506-2553 (2008); Gross, etal., Bioanalysis, 8(19):2071-2089 (2016); Tan, et al., Angew. Chem. Int. Ed., 53(37):9822-9826 (2014); Miao, et al., Anal. Chem., 75(21):5825-5834 (2003); Li, et al., Sensor Actuat. B-Chem., 210:468-474 (2015); Li, et al., Anal. Chem., 89(1):358-371 (2017); Xu, et al., Chem. Commun., 50(65):9097-9100 (2014); Lv, etal., J. Am. Chem. Soc., 140(8):2801-2804 (2018); Zhou, et al., Anal. Chem., 90(16):10024-10030 (2018); Zhao, et al., Anal. Chem., 91(3):1989-1996 (2019)).

ECL in the near infrared (near-IR) range has received far less attention compared to near-IR fluorescence (Ding, et al., Science, 296:1293-1296 (2002)). It is considered challenging to enhance ECL signal due to the complex reactions involved in ECL generation. Therefore, the low background noise in the near-IR range is significant for ECL to achieve a high signal/noise ratio. There are two types of pathways to generate ECL, annihilation pathways and coreactant pathways. Since the introduction of the coreactant pathway, specifically the seminal work of tripropyl-amine (TPrA) as coreactant to enhance the oxidative reduction ECL of tris(bipyridine)ruthenium(II) complex (Ru(bpy)₃), the Ru(bpy)₃-TPrA system has been the predominant option for real applications and as a standard to evaluate new ECL reagents/system (Miao, et al., J. Am. Chem. Soc., 124:14478-14485 (2002)). However, the addition of millimolar to sub-molar coreactants complicates the detection system and can greatly limit the applicability in both basic research and practical applications. An annihilation ECL results from the self-quenching of radical species generated from the oxidation and reduction of the same ECL reagents, which is better suited for studies of fundamental mechanisms but not real applications due to the low signals compared to the coreactant mechanism (Lee, et al., ACS Appl. Mater. Interfaces, 10(48):41562-41569 (2018)).

There remains a need to develop electrochemiluminescence (ECL) sensors that have high signal output and high signal/noise ratio. There is also a need for an effective sensing method using ECL that is rapid, simple, and/or allows for sensitive and specific detection of analytes of interest at a low cost.

Therefore, it is the object of the present invention to provide electrochemiluminescence sensors.

It is another object of the present invention to provide methods of making such electrochemiluminescence sensors.

It is another object of the present invention to provide methods of using such electrochemiluminescence sensors.

It is yet another object of the present invention to provide kits for detection using such electrochemiluminescence sensors.

### SUMMARY OF THE INVENTION

Disclosed are sensors and methods using electrochemiluminescence (ECL). In particular, disclosed are ECL sensors containing metal nanoclusters. In some instances, the metal nanoclusters can be organo-soluble or aqueous soluble. In some instances, the metal nanoclusters can be organo-soluble. In some instances, the metal nanoclusters can be aqueous soluble.

In some instances, the metal nanoclusters contain a metal core and a plurality of ligands. In some instances, the metal core of the metal nanoclusters can contain metal atoms or a mixture of metal atoms. In some instances, the metal core of the metal nanoclusters can contain metal atoms. In some instances, the metal core of the metal nanoclusters can contain a mixture of metal atoms. In some instances, the ligands of the metal nanoclusters can contain thiolates, phosphines, other non-metallic elements, or combinations thereof. In some instances, the ligands of the metal nanoclusters can contain thiolates, phosphines, halogens, or combinations thereof. In some instances, the ligands of the metal nanoclusters can contain thiolates. In some instances, the ligands of the metal nanoclusters can contain phosphines. In some instances, the ligands of the metal nanoclusters can contain halogens. In some instances, the ligands of the metal nanoclusters can contain a mixture of thiolates and halogens. In some instances, the ligands of the metal nanoclusters can contain a mixture of phosphines and halogens. In some instances, the ligands of the metal nanoclusters can contain a mixture of thiolates and phosphines. In some instances, the ligands of the metal nanoclusters can contain a mixture of thiolates, phosphines, and halogens.

In some instances, the metal atoms of the core are gold, silver, aluminum, tin, magnesium, copper, nickel, iron, cobalt, magnesium, platinum, palladium, iridium, vanadium, rhodium, or ruthenium. In some instances, the metal atoms of the core are gold. In some instances, the mixture of metal atoms of the core contains gold and silver. In some instances, the mixture of metal atoms of the core contains 12 gold atoms and 13 silver atoms.

In some instances, the metal nanoclusters further contain targeting moieties bound to the core and/or the ligands of the metal nanoclusters. In some instances, the targeting moieties can bound to the core of the metal nanoclusters. In some instances, the targeting moieties can bound to the ligands of the metal nanoclusters. In some instances, the targeting moieties can bound to both the core and the ligands of the metal nanoclusters.

In some instances, the ECL sensor further contains a conductive substrate. In some instances, the metal nanoclusters can be assembled on the surface of the conductive substrate. In some instances, the metal nanoclusters can be assembled on the surface of the conductive substrate in the form of a film.

In some instances, the ECL sensor can further contain coreactants. In some instances, the coreactant can be associated with the metal nanoclusters covalently or non-covalently. In some instances, the coreactants can be associated with the metal nanoclusters covalently. In some instances, the coreactants can be associated with the metal nanoclusters non-covalently. In some instances, the coreactants can be amines, oxalates, persulfates, hydrogen peroxide, nitrile, unsubstituted cyano, substituted cyano, unsubstituted benzophenone, substituted benzophenone, unsubstituted benzoic acid, substituted benzoic acid, unsubstituted naphthalene, substituted naphthalene, unsubstituted biphenyl, or substituted biphenyl. In some instances, the coreactant is an amine. In some instances, the coreactant is a tertiary amine.

In some instances, the metal nanoclusters can display near-IR ECL. In some instances, the metal nanoclusters can display ECL higher than tris(bipyridine)ruthenium(II) complex (Rubpy) under the same conditions.

In some instances, the metal nanoclusters can be rod-shaped metal nanoclusters.

An ECL sensing array containing two or more ECL sensors is also disclosed.

The ECL sensors disclosed herein can be utilized in a method of testing the presence, absence, or concentration of an analyte of interest in a sample. The method includes: (i) contacting the sample with the ECL sensor, (ii) applying a potential to the ECL sensor, and (iii) detecting the ECL and/or a redox current of the metal nanoclusters.

The ECL sensors of an ECL sensing array can be utilized in a method of screening the presence, absence, or concentration of a plurality of analytes of interest in a sample. The method includes: (i) contacting the sample with the ECL sensors of the ECL sensing array, (ii) applying a potential to each of the ECL sensors, and (iii) detecting the ECL and/or redox currents of the metal nanoclusters.

In some instances, the potential applied (such as in the method of screening for the presence, absence, or concentration of a plurality of analytes of interest in a sample) can be the same or different for each ECL sensor in the ECL sensing array. In some instances, the potential applied can be the same for each ECL sensor in the ECL sensing array. In some instances, the potential applied can be different for each ECL sensor in the ECL sensing array. In some instances, a first potential can be applied for two or more ECL sensors in the ECL sending array and a second potential that is different from the first potential can be applied for one or more ECL sensors in the ECL array.

In some instances, the potential applied in the disclosed methods can be by linear sweeping from a first potential to a second potential, cyclic sweeping between a first potential and a second potential, or stepping between a first potential to a second potential. In some instances, the potential can be applied by linear sweeping from a first potential to a second potential. In some instances, the potential can be applied by cyclic sweeping between a first potential and a second potential. In some instances, the potential can be applied by stepping between a first potential to a second potential.

In some instances, the applied potential can be sufficient to provide enough energy to activate the corresponding energy states of the metal nanoclusters, the coreactant, the analyte, or combinations thereof. In some instances, the applied potential can be sufficient to provide enough energy to activate the corresponding energy states of the metal nanoclusters. In some instances, the applied potential can be sufficient to provide enough energy to activate the corresponding energy states of the coreactants. In some instances, the applied potential can be sufficient to provide enough energy to activate the corresponding energy states of the analytes. In some instances, the applied potential can be sufficient to provide enough energy to activate the corresponding energy states of the metal nanoclusters and the coreactants. In some instances, the applied potential can be sufficient to provide enough energy to activate the corresponding energy states of the metal nanoclusters and the analytes. In some instances, the applied potential can be sufficient to provide enough energy to active the corresponding energy states of the metal nanoclusters, the coreactants, and the analytes.

In some instances, the analytes can interact with the metal nanoclusters and/or the coreactant. In some instances, the analytes can interact with the metal nanoclusters. In some instances, the analytes can interact with the coreactants. In some instances, the analytes can interact with the metal nanoclusters and the coreactants.

In some instances, the ECL of the metal nanoclusters can increase or decrease upon an interaction between the analyte and the metal nanoclusters and/or the coreactant as compared to the ECL of the metal nanoclusters in the absence of the analyte. In some instances, the ECL of the metal nanoclusters can increase or decrease upon an interaction between the analyte and the metal nanoclusters as compared to the ECL of the metal nanoclusters in the absence of the analyte. In some instances, the ECL of the metal nanoclusters can increase or decrease upon an interaction between the analyte, the metal nanoclusters, and the coreactant as compared to the ECL of the metal nanoclusters in the absence of the analyte. In some instances, the level of increase or decrease of the ECL of the metal nanoclusters can be correlated to the concentration and/or amount of the analyte.

In some instances, the redox current of the metal nanoclusters can increase or decrease upon an interaction between the analyte and the metal nanoclusters and/or the coreactant as compared to the redox current of the metal nanoclusters in the absence of the analyte. In some instances, the redox current of the metal nanoclusters can increase or decrease upon an interaction between the analyte and the metal nanoclusters as compared to the redox current of the metal nanoclusters in the absence of the analyte. In some instances, the redox current of the metal nanoclusters can increase or decrease upon an interaction between the analyte, the metal nanoclusters, and the coreactant as compared to the redox current of the metal nanoclusters in the absence of the analyte. In some instances, the level of increase or decrease of the redox current of the metal nanoclusters can be correlated to the concentration and/or amount of the analyte.

In some instances, the sample can be a buffer solution, a biological sample, or a combination of both. In some instances, the sample can be a buffer solution. In some instances, the sample can be a biological sample. In some instances, the sample can be a combination of buffer solution and biological sample. In some instances, the biological sample can be a bodily fluid or mucus selected from the group consisting of saliva, sputum, tear, sweat, urine, exudate, blood, serum, plasma, and vaginal discharge.

In some instances, the analyte can be a drug, metabolite, biomarker, metal ion, or combinations thereof. In some instances, the analyte can be a drug. In some instances, the analyte can be a piperazine derivative drug.

In some instances, the ECL of the metal nanoclusters can be detected by a photon detector. Photon detectors are known and commercially available (e.g., camera). In some instances, the ECL of the metal nanoclusters can be detected by a camera.

### BRIEF DESCRIPTION OF THE DRAWINGS

FIGS. 1A-1D are graphs showing the cyclic voltammograms (left axis, curve 1) and ECL-potential curves (right axis, curve 2) of Au₁₂Ag₁₃. Arrows on the cyclic voltammograms (CV) indicate the initial scan direction. The Au₁₂Ag₁₃ concentration is ca. ˜10 μM in 1:1 toluene:acetonitrile (TOL:ACN) with 0.1 M TBAP electrolyte. Potential scan rate is 0.1 V/s. FIG. 1A shows the first cycle of the CV and ECL-potential curve with the first segment scanned in the positive direction. FIG. 1B shows the second cycle of the CV and ECL-potential curve with the first segment scanned in the positive direction. FIG. 1C shows the first cycle of the CV and ECL-potential curve with the first segment scanned in the negative direction. FIG. 1D shows the second cycle of the CV and ECL-potential curve with the first segment scanned in the negative direction.

FIGS. 2A and 2B are graphs showing the differential pulse voltammogram (DPV) (FIG. 2A) and CV (FIG. 2B) of 1 mM Au₁₂Ag₁₃ in 1:1 TOL:ACN solution at room temperature, with 0.1 M tetra-n-butylammonium perchlorate (TBAP) as supporting electrolyte, respectively. The DPV and CV were recorded at a Pt disk electrode (d ˜0.5 mm) as working electrode. An Ag/AgCl wire and a Pt foil were used as reference and counter electrodes. A 20 min purging with Ar process was executed before measurement. FIG. 2C is a graph showing the photon energy spectrum of Au₁₂Ag₁₃.

FIGS. 3A-3D are graphs showing the self-annihilation ECL of Au₁₂Ag₁₃ generated by potential step. The Au₁₂Ag₁₃ concentration is ca. ˜10 μM in 1:1 TOL:ACN with 0.1 M TBAP electrolyte. FIG. 3A shows the ECL profile under potential steps between −1.2 V and +1.0 V. The electrode potential was held for 5 s at the denoted potentials and stepped cyclically (3 cycles shown). No potential was applied in the first and final 5 s. FIG. 3B is in log scale for the ECL intensity to better illustrate the gradual decay. FIG. 3C shows the first 0.3 s data points of the ECL peak after the potential step at +1.0 V for three cycles in log scale. FIG. 3D shows the first 0.3 s data points of the ECL peak after the potential step at −1.2 V for three cycles in linear scale. The data sampling rate is 13.3 ms determined by the camera exposure time.

FIG. 4 are graphs showing the comparison of Au₁₂Ag₁₃ CVs measured before and after bulk electrolysis at −1.2 V. The potential range is limited within the positive region (0 to 1.1 V, curves 1 and 2) and the negative region (0 to −1.2 V, curves 3 and 4) respectively. Both curves before electrolysis are control groups (curves 2 and 4). A new peak appears at +0.7 V in the first segment and decreases in the following cycles (curve 1). The Au₁₂Ag₁₃ concentration is ca. ˜1 mM in 1:1 TOL:ACN with 0.1 M TBAP electrolyte.

FIG. 5 are graphs showing the CVs of Au₁₂Ag₁₃ on positive (0 to 1.1 V, curves 1 and 2) and negative (0 to −1.2 V, curves 3 and 4) regions before and after bulk electrolysis at +1.1 V. Both of the before electrolysis curves are control groups (curves 2 and 4). After electrolysis, there is no big difference on either the negative or positive region comparing to before electrolysis.

FIGS. 6A and 6B are graphs showing the self-annihilation ECL reaction pathways for the oxidative (FIG. 6A) and reductive (FIG. 6B) ECL and the corresponding energy states. The numbers 1 and 2 in FIGS. 6A and 6B indicate the order of potential steps, with applied potential from negative to positive and from positive to negative respectively.

FIG. 7 is a graph showing the ECL profiles after the excitation of the HOMO state of Au₁₂Ag₁₃, the LUMO state of Au₁₂Ag₁₃, and none. The Au₁₂Ag₁₃ concentration is ca. about 10 μM in 1:1 TOL:ACN with 0.1 M TBAP electrolyte. The electrode potential was held for 5 s at the denoted potentials and stepped cyclically (3 cycles shown). No potential was applied in the first and final 5 s.

FIG. 8A is a graph showing the step-annihilation ECL of 10 μM Au₁₂Ag₁₃ and 10 μM Rubpy without coreactants at denoted potentials respectively. The electrode potential is held for 5 s in each step over three cycles. The first 5 s and last 5 s provide the baseline. FIG. 8B is in log scale for the ECL intensity to better illustrate the gradual decay. FIG. 8C are graphs showing the CVs of Rubpy (curve 1), TPrA (curve 2), and Rubpy with TPrA (curve 3) in 1:1 TOL/ACN with 0.1 M TBAP at a 0.1 V/s scan rate respectively. The CV was recorded at a Pt disk electrode (d 0.5 mm) as working electrode. An Ag/AgCl wire and a Pt foil were used as reference and counter electrodes. A 20 min purging with Ar process was executed before measurement. FIG. 8D are graphs showing the CV (left axis, curve 1) and ECL-potential (right axis, curve 2) curves for Rubpy-only. For CV and ECL measurements, a Pt mesh was used as working electrode in a 20 mL cuvette and purging with Ar. The purging process is continued to 20 min before testing. The concentration of Rubpy is ca. ˜10 μM. The supporting electrolyte is 0.1 M TBAP. The potential scan rate is 0.1 V/s. FIG. 8E are graphs showing the coreactant oxidative-reduction ECL of 10 μM Au₁₂Ag₁₃ and 10 μM Rubpy. Different TPrA coreactant concentrations are used in the Rubpy measurements as multi-point standard/reference.

FIGS. 9A and 9B are graphs showing the ECL profiles of [AgₓAu₂₅₋ₓ(PPh₃)₁₀(SC₂H₄Ph)₅Cl₂]²⁺ (x≤13) nanoclusters (FIG. 9A) and Au₂₅(SC₂H₄Ph)₁₈⁻ nanoclusters (FIG. 9B) respectively. The only appreciable ECL signals (curve 1) were recorded after the excitation of both the HOMO and LUMO states via electron transfer reactions. Other curves are LUMO activation (curve 2), HOMO activation (curve 3) and none (curve 4; the electrode potential was held within the HOMO & LUMO states and insufficient to drive electron transfer reactions). The concentration of the nanoclusters is ca. about 10 μM in 1:1 TOL:ACN with 0.1 M TBAP electrolyte. The electrode potential was held for 5 s at the denoted potentials and stepped cyclically (3 cycles shown). No potential was applied in the first and final 5 s. The camera exposure time is 13.3 ms (same as one in Au₁₂Ag₁₃ tests) for the measurements of [AgₓAu₂₅₋ₓ(PPh₃)₁₀(SC₂H₄Ph)₅Cl₂]²⁺ (×≤13) nanoclusters. Longer exposure time (50 ms) was used to collect the signal with adequate/comparable signal/noise ratio for the measurements of Au₂₅(SC₂H₄Ph)₁₈⁻ nanoclusters.

FIG. 10 are graphs showing the ECL (left axis, curve 1) and PL (right axis, curve 2) spectra of Au₁₂Ag₁₃. The ECL spectrum was collected with 10 μM Au₁₂Ag₁₃ and 1 mM TPrA under +1.0V. The PL sample is generally about ten times less concentrated and without TPrA. The PL spectrum profile was corrected.

FIG. 11 is a graph showing the UV-vis spectrum of Au₁₂Ag₁₃ in 1:1 TOL/ACN.

FIG. 12 is a graph showing the self-annihilation ECL profile of [Ag₁₃Au₁₂(PPh₃)₁₀(SC₂H₄Ph)₅Cl₂]²⁺ nanoclusters assembled on ITO electrode measured from 10 cycles of potential stepping in linear scale. The ECL of Ag₁₃Au₁₂ film on ITO was measured in PBS at pH 7.4 under ambient condition. The potential was stepped every 0.2 seconds between −1.0 V and 0.9 V. The first and last 0.2 s was plotted as a baseline.

FIG. 13 is a graph showing the self-annihilation ECL profile of [Ag₁₃Au₁₂(PPh₃)₁₀(SC₂H₄Ph)₅Cl₂]²⁺ nanoclusters assembled on ITO electrode measured from 240 cycles of potential stepping in log scale. The ECL of Ag₁₃Au₁₂ film on ITO was measured in PBS at pH 7.4 under ambient condition. The potential was stepped every 0.2 seconds between −1.0 V and 0.9 V. The first and last 1 second was plotted as a baseline.

FIG. 14 is a graph showing the fluorescence emission intensities in an area at the edge of the NC film (Border) and an interior area of the NC film (Interior). NC film was prepared by spin-coating NCs in a 1:1 DCM:chloroform mixed solvent.

FIGS. 15A and 15B are graphs showing the CV-ECL curves of Ag₁₃Au₁₂ assembled on ITO working electrode tested without (FIG. 15A) and with (FIG. 15B) 1 mM cetirizine in PBS pH 7.4. Cyclic voltammogram current is the curve on top and ECL intensity is the curve on the bottom.

FIG. 16A is a graph showing ECL signals with and without 1 mM cetirizine in PBS pH 7.4. The electrode potential was held for 0.3 s at −1.0 V (starting from 0 second) and then stepped to 1.0 V for 0.1 s cyclically. Data with four repeated cycles are plotted.

The NCs were assembled on ITO by spin-coating NCs in DCM. FIG. 16B is a zoom-in view of the ECL signals generated in the 2^{nd}, 3^{rd} and 4^{th} cycles.

FIG. 17 is a graph showing ECL profile of the NCs microcrystals in 1 mM cetirizine and without cetirizine in pH 7.4 PBS buffer. The electrode potential was held for 0.3 s at −1.0 V and then step to 1.0 V for 0.1 s cyclically. The NCs were assembled on ITO by spin-coating NCs in DCM.

### DETAILED DESCRIPTION OF THE INVENTION

The disclosed sensors, metal nanoclusters, kits, and methods can be understood more readily by reference to the following detailed description of particular embodiments and the Examples included therein and to the Figures and their previous and following description.

All methods described herein can be performed in any suitable order unless otherwise indicated herein or otherwise clearly contradicted by context. The use of any and all examples or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The disclosed sensors, metal nanoclusters, and kits, can be used for, can be used in conjunction with, can be used in preparation for, or are products of the disclosed methods. It is understood that when combinations, subsets, interactions, groups, etc. of these sensors, metal nanocluster, and kits are disclosed, while specific reference of each various individual and collective combinations of these materials may not be explicitly disclosed, each is specifically contemplated and described herein. For example, if a metal nanocluster is disclosed and discussed and a number of modifications that can be made to a number of molecules including the metal nanoclusters are discussed, each and every combination and permutation of the metal nanoclusters and the modifications that are possible are specifically contemplated unless specifically indicated to the contrary.

Further, each of the sensors, metal nanoclusters, kits, components, etc. contemplated and disclosed herein can also be specifically and independently included or excluded from any group, subgroup, list, set, etc. of such materials. These concepts apply to all aspects of this application including, but not limited to, steps in methods of making and using the disclosed sensors, metal nanoclusters, components, and kits. Thus, if there are a variety of additional steps that can be performed it is understood that each of these additional steps can be performed with any specific embodiment or combination of embodiments of the disclosed methods, and that each such combination is specifically contemplated and should be considered disclosed.

### I. Definitions

The singular forms “a,” “an,” and “the” include plural reference unless the context clearly dictates otherwise. For example, reference to “a compound” includes a plurality of compounds and reference to “the compound” is a reference to one or more compounds and equivalents thereof known to those skilled in the art.

The terms “can,” and “can be,” and related terms are intended to convey that the subject matter involved is optional (that is, the subject matter is present in some forms and is not present in other forms), not a reference to a capability of the subject matter or to a probability, unless the context clearly indicates otherwise.

The terms “optional” and “optionally” mean that the subsequently described event, circumstance, or material may or may not occur or be present, and that the description includes instances where the event, circumstance, or material occurs or is present and instances where it does not occur or is not present.

As used herein, the term “derivative” refers to a compound that possesses the same core as a parent compound, but differs from the parent compound in bond order, in the absence or presence of one or more atoms and/or groups of atoms, and combinations thereof. The derivative can differ from the parent compound, for example, in one or more substituents present on the core, which may include one or more atoms, functional groups, or substructures. The derivative can also differ from the parent compound in the bond order between atoms within the core. In general, a derivative can be formed, at least theoretically, from the parent compound via chemical and/or physical processes.

As used herein, the term “substituted,” means that the chemical group or moiety contains one or more substituents replacing the hydrogen atoms in the chemical group or moiety. The substituents include, but are not limited to:

a halogen atom, an alkyl group, a cycloalkyl group, a heteroalkyl group, a cycloheteroalkyl group, an alkenyl group, a heteroalkenyl group, an alkynyl group, a heteroalkynyl group, an aryl group, a heteroaryl group, a polyaryl group, a polyheteroaryl group, —OH, —SH, —NH₂, —N₃, —OCN, —NCO, —ONO₂, —CN, —NC, —ONO, —CONH₂, —NO, —NO₂, —ONH₂, —SCN, —SNCS, —CF₃, —CH₂CF₃, —CH₂Cl, —CHCl₂, —CH₂NH₂, —NHCOH, —CHO, —COCl, —COF, —COBr, —COOH, —SO₃H, —CH₂SO₂CH₃, —PO₃H₂, —OPO₃H₂, —P(═O)(OR^{T1′})(OR^{T2′}), —OP(═O)(OR^{T1′})(OR^{T2′}), —BR^{T1′}(OR^{T2′}), —B(OR^{T1′})(OR^{T2′}), or -G′R^{T1′} in which -T′ is —O—, —S—, —NR^{T2′}—, —C(═O)—, —S(═O)—, —SO₂—, —C(═O)O—, —C(═O)NR^{T2′}—, —OC(═O)—, —NR^{T2′}C(═O)—, —OC(═O)O—, —OC(═O)NR^{T2′}—, —NR^{T2′}C(═O)O—, —NR^{T2′}C(═O)NR^{T3′}—, —C(═S)—, —C(═S)S—, —SC(═S)—, —SC(═S)S—, —C(═NR^{T2′})—, —C(═NR^{T2′})O—, —C(═NR^{T2′})NR^{T3′}—, —OC(═NR^{T2′})—, —NR^{T2′}C(═NR^{T3′})—, —NR^{T2′}SO₂—, —C(═NR^{T2′})NR^{T3′}—, —OC(═NR^{T2′})—, —NR^{T2′}C(═NR^{T3′})—, —NR^{T2′}SO₂—, —NR^{T2′}SO₂NR^{T3′}—, —NR^{T2′}C(═S)—, —SC(═S)NR^{T2′}—, —NR^{T2′}C(═S)S—, —NR^{T2′}C(═S)NR^{T3′}—, —SC(═NR^{T2′})—, —C(═S)NR^{T2′}—, —OC(═S)NR^{T2′}—, —NR^{T2′}C(═S)O—, —SC(═O)NR^{T2′}—, —NR^{T2′}C(═O)S—, —C(═O)S—, —SC(═O)—, —SC(═O)S—, —C(═S)O—, —OC(═S)—, —OC(═S)O—, —SO₂NR^{T2′}—, —BR^{T2′}—, or —PR^{T2′}—; where each occurrence of R^{T1′}, R^{T2′}, and R^{T3′} is, independently, a hydrogen atom, a halogen atom, an alkyl group, a heteroalkyl group, an alkenyl group, a heteroalkenyl group, an alkynyl group, a heteroalkynyl group, an aryl group, or a heteroaryl group.

In some instances, “substituted” also refers to one or more substitutions of one or more of the carbon atoms in a carbon chain (e.g., alkyl, alkenyl, alkynyl, and aryl groups) by a heteroatom, such as, but not limited to, nitrogen, oxygen, and sulfur.

It is understood that “substitution” or “substituted” includes the implicit proviso that such substitution is in accordance with permitted valence of the substituted atom and the substituent, and that the substitution results in a stable compound, i.e., a compound that does not spontaneously undergo transformation such as by rearrangement, cyclization, elimination, etc.

As used herein, the term “alkyl” refers to univalent groups derived from alkanes by removal of a hydrogen atom from any carbon atom. Alkanes represent saturated hydrocarbons, including those that are cyclic (either monocyclic or polycyclic). Alkyl groups can be linear or branched. “Cycloalkyl group” refers to an alkyl group that is cyclic. Preferred alkyl groups have one to 30 carbon atoms, i.e., C₁-C₃₀ alkyl. In some forms, a C₁-C₃₀ alkyl can be a linear C₁-C₃₀ alkyl, a branched C₁-C₃₀ alkyl, or a linear or branched C₁-C₃₀ alkyl. More preferred alkyl groups have one to 20 carbon atoms, i.e., C₁-C₂₀ alkyl. In some forms, a C₁-C₂₀ alkyl can be a linear C₁-C₂₀ alkyl, a branched C₁-C₂₀ alkyl, or a linear or branched C₁-C₂₀ alkyl. Still more preferred alkyl groups have one to 10 carbon atoms, i.e., C₁-C₁₀ alkyl. In some forms, a C₁-C₁₀ alkyl can be a linear C₁-C₁₀ alkyl, a branched C₁-C₁₀ alkyl, or a linear or branched C₁-C₁₀ alkyl. The most preferred alkyl groups have one to 6 carbon atoms, i.e., C₁-C₆ alkyl. In some forms, a C₁-C₆ alkyl can be a linear C₁-C₆ alkyl, a branched C₁-C₆ alkyl, or a linear or branched C₁-C₆ alkyl. Preferred C₁-C₆ alkyl groups have one to four carbons, i.e., C₁-C₄ alkyl. In some forms, a C₁-C₄ alkyl can be a linear C₁-C₄ alkyl, a branched C₁-C₄ alkyl, or a linear or branched C₁-C₄ alkyl. Any C₁-C₃₀ alkyl, C₁-C₂₀ alkyl, C₁-C₁₀ alkyl, C₁-C₆ alkyl, and/or C₁-C₄ alkyl groups can, alternatively, be cyclic. If the alkyl is branched, it is understood that at least four carbons are present. If the alkyl is cyclic, it is understood that at least three carbons are present.

As used herein, the term “heteroalkyl” refers to alkyl groups where one or more carbon atoms are replaced with a heteroatom, such as, O, N, or S. Heteroalkyl groups can be linear or branched. “Cycloheteroalkyl group” refers to a heteroalkyl group that is cyclic. Preferred heteroalkyl groups have one to 30 carbon atoms, i.e., C₁-C₃₀ heteroalkyl. In some forms, a C₁-C₃₀ heteroalkyl can be a linear C₁-C₃₀ heteroalkyl, a branched C₁-C₃₀ heteroalkyl, or a linear or branched C₁-C₃₀ heteroalkyl. More preferred heteroalkyl groups have one to 20 carbon atoms, i.e., C₁-C₂₀ heteroalkyl. In some forms, a C₁-C₂₀ heteroalkyl can be a linear C₁-C₂₀ heteroalkyl, a branched C₁-C₂₀ heteroalkyl, or a linear or branched C₁-C₂₀ heteroalkyl. Still more preferred heteroalkyl groups have one to 10 carbon atoms, i.e., C₁-C₂₀ heteroalkyl. In some forms, a C₁-C₁₀ heteroalkyl can be a linear C₁-C₁₀ heteroalkyl, a branched C₁-C₁₀ heteroalkyl, or a linear or branched C₁-C₁₀ heteroalkyl. The most preferred heteroalkyl groups have one to 6 carbon atoms, i.e., C₁-C₆ heteroalkyl. In some forms, a C₁-C₆ heteroalkyl can be a linear C₁-C₆ heteroalkyl, a branched C₁-C₆ heteroalkyl, or a linear or branched C₁-C₆ heteroalkyl. Preferred C₁-C₆ heteroalkyl groups have one to four carbons, i.e., C₁-C₄ heteroalkyl. In some forms, a C₁-C₄ heteroalkyl can be a linear C₁-C₄ heteroalkyl, a branched C₁-C₄ heteroalkyl, or a linear or branched C₁-C₄ heteroalkyl. If the heteroalkyl is branched, it is understood that at least four carbons are present. If the heteroalkyl is cyclic, it is understood that at least three carbons are present.

As used herein, the term “alkenyl” refers to univalent groups derived from alkenes by removal of a hydrogen atom from any carbon atom. Alkenes are unsaturated hydrocarbons that contain at least one carbon-carbon double bond. Alkenyl groups can be linear, branched, or cyclic. Preferred alkenyl groups have two to 30 carbon atoms, i.e., C₂-C₃₀ alkenyl. In some forms, a C₂-C₃₀ alkenyl can be a linear C₂-C₃₀ alkenyl, a branched C₂-C₃₀ alkenyl, a cyclic C₂-C₃₀ alkenyl, a linear or branched C₂-C₃₀ alkenyl, a linear or cyclic C₂-C₃₀ alkenyl, a branched or cyclic C₂-C₃₀ alkenyl, or a linear, branched, or cyclic C₂-C₃₀ alkenyl. More preferred alkenyl groups have two to 20 carbon atoms, i.e., C₂-C₂₀ alkenyl. In some forms, a C₂-C₂₀ alkenyl can be a linear C₂-C₂₀ alkenyl, a branched C₂-C₂₀ alkenyl, a cyclic C₂-C₂₀ alkenyl, a linear or branched C₂-C₂₀ alkenyl, a branched or cyclic C₂-C₂₀ alkenyl, or a linear, branched, or cyclic C₂-C₂₀ alkenyl. Still more preferred alkenyl groups have two to 10 carbon atoms, i.e., C₂-C₁₀ alkenyl. In some forms, a C₂-C₁₀ alkenyl can be a linear C₂-C₁₀ alkenyl, a branched C₂-C₁₀ alkenyl, a cyclic C₂-C₁₀ alkenyl, a linear or branched C₂-C₁₀ alkenyl, a branched or cyclic C₂-C₁₀ alkenyl, or a linear, branched, or cyclic C₂-C₂₀ alkenyl. The most preferred alkenyl groups have two to 6 carbon atoms, i.e., C₂-C₆ alkenyl. In some forms, a C₂-C₆ alkenyl can be a linear C₂-C₆ alkenyl, a branched C₂-C₆ alkenyl, a cyclic C₂-C₆ alkenyl, a linear or branched C₂-C₆ alkenyl, a branched or cyclic C₂-C₆ alkenyl, or a linear, branched, or cyclic C₂-C₆ alkenyl. Preferred C₂-C₆ alkenyl groups have two to four carbons, i.e., C₂-C₄ alkenyl. In some forms, a C₂-C₄ alkenyl can be a linear C₂-C₄ alkenyl, a branched C₂-C₄ alkenyl, a cyclic C₂-C₄ alkenyl, a linear or branched C₂-C₄ alkenyl, a branched or cyclic C₂-C₄ alkenyl, or a linear, branched, or cyclic C₂-C₄ alkenyl. If the alkenyl is branched, it is understood that at least four carbons are present. If the alkenyl is cyclic, it is understood that at least three carbons are present.

As used herein, the term “heteroalkenyl” refers to alkenyl groups in which one or more doubly bonded carbon atoms are replaced by a heteroatom. Heteroalkenyl groups can be linear, branched, or cyclic. Preferred heteroalkenyl groups have two to 30 carbon atoms, i.e., C₂-C₃₀ heteroalkenyl. In some forms, a C₂-C₃₀ heteroalkenyl can be a linear C₂-C₃₀ heteroalkenyl, a branched C₂-C₃₀ heteroalkenyl, a cyclic C₂-C₃₀ heteroalkenyl, a linear or branched C₂-C₃₀ heteroalkenyl, a linear or cyclic C₂-C₃₀ heteroalkenyl, a branched or cyclic C₂-C₃₀ heteroalkenyl, or a linear, branched, or cyclic C₂-C₃₀ heteroalkenyl. More preferred heteroalkenyl groups have two to 20 carbon atoms, i.e., C₂-C₂₀ heteroalkenyl. In some forms, a C₂-C₂₀ heteroalkenyl can be a linear C₂-C₂₀ heteroalkenyl, a branched C₂-C₂₀ heteroalkenyl, a cyclic C₂-C₂₀ heteroalkenyl, a linear or branched C₂-C₂₀ heteroalkenyl, a branched or cyclic C₂-C₂₀ heteroalkenyl, or a linear, branched, or cyclic C₂-C₂₀ heteroalkenyl. Still more preferred heteroalkenyl groups have two to 10 carbon atoms, i.e., C₂-C₁₀ heteroalkenyl. In some forms, a C₂-C₁₀ heteroalkenyl can be a linear C₂-C₁₀ heteroalkenyl, a branched C₂-C₁₀ heteroalkenyl, a cyclic C₂-C₁₀ heteroalkenyl, a linear or branched C₂-C₁₀ heteroalkenyl, a branched or cyclic C₂-C₁₀ heteroalkenyl, or a linear, branched, or cyclic C₂-C₂₀ heteroalkenyl. The most preferred heteroalkenyl groups have two to 6 carbon atoms, i.e., C₂-C₆ heteroalkenyl. In some forms, a C₂-C₆ heteroalkenyl can be a linear C₂-C₆ heteroalkenyl, a branched C₂-C₆ heteroalkenyl, a cyclic C₂-C₆ heteroalkenyl, a linear or branched C₂-C₆ heteroalkenyl, a branched or cyclic C₂-C₆ heteroalkenyl, or a linear, branched, or cyclic C₂-C₆ heteroalkenyl. Preferred C₂-C₆ heteroalkenyl groups have two to four carbons, i.e., C₂-C₄ heteroalkenyl. In some forms, a C₂-C₄ heteroalkenyl can be a linear C₂-C₄ heteroalkenyl, a branched C₂-C₄ heteroalkenyl, a cyclic C₂-C₄ heteroalkenyl, a linear or branched C₂-C₄ heteroalkenyl, a branched or cyclic C₂-C₄ heteroalkenyl, or a linear, branched, or cyclic C₂-C₄ heteroalkenyl. If the heteroalkenyl is branched, it is understood that at least four carbons are present. If heteroalkenyl is cyclic, it is understood that at least three carbons are present.

As used herein, the term “alkynyl” refers to univalent groups derived from alkynes by removal of a hydrogen atom from any carbon atom. Alkynes are unsaturated hydrocarbons that contain at least one carbon-carbon triple bond. Alkynyl groups can be linear, branched, or cyclic. Preferred alkynyl groups have two to 30 carbon atoms, i.e., C₂-C₃₀ alkynyl. In some forms, a C₂-C₃₀ alkynyl can be a linear C₂-C₃₀ alkynyl, a branched C₂-C₃₀ alkynyl, a cyclic C₂-C₃₀ alkynyl, a linear or branched C₂-C₃₀ alkynyl, a linear or cyclic C₂-C₃₀ alkynyl, a branched or cyclic C₂-C₃₀ alkynyl, or a linear, branched, or cyclic C₂-C₃₀ alkynyl. More preferred alkynyl groups have two to 20 carbon atoms, i.e., C₂-C₂₀ alkynyl. In some forms, a C₂-C₂₀ alkynyl can be a linear C₂-C₂₀ alkynyl, a branched C₂-C₂₀ alkynyl, a cyclic C₂-C₂₀ alkynyl, a linear or branched C₂-C₂₀ alkynyl, a branched or cyclic C₂-C₂₀ alkynyl, or a linear, branched, or cyclic C₂-C₂₀ alkynyl. Still more preferred alkynyl groups have two to 10 carbon atoms, i.e., C₂-C₁₀ alkynyl. In some forms, a C₂-C₁₀ alkynyl can be a linear C₂-C₁₀ alkynyl, a branched C₂-C₁₀ alkynyl, a cyclic C₂-C₁₀ alkynyl, a linear or branched C₂-C₁₀ alkynyl, a branched or cyclic C₂-C₁₀ alkynyl, or a linear, branched, or cyclic C₂-C₂₀ alkynyl. The most preferred alkynyl groups have two to 6 carbon atoms, i.e., C₂-C₆ alkynyl. In some forms, a C₂-C₆ alkynyl can be a linear C₂-C₆ alkynyl, a branched C₂-C₆ alkynyl, a cyclic C₂-C₆ alkynyl, a linear or branched C₂-C₆ alkynyl, a branched or cyclic C₂-C₆ alkynyl, or a linear, branched, or cyclic C₂-C₆ alkynyl. Preferred C₂-C₆ alkynyl groups have two to four carbons, i.e., C₂-C₄ alkynyl. In some forms, a C₂-C₄ alkynyl can be a linear C₂-C₄ alkynyl, a branched C₂-C₄ alkynyl, a cyclic C₂-C₄ alkynyl, a linear or branched C₂-C₄ alkynyl, a branched or cyclic C₂-C₄ alkynyl, or a linear, branched, or cyclic C₂-C₄ alkynyl. If the alkynyl is branched, it is understood that at least four carbons are present. If alkynyl is cyclic, it is understood that at least three carbons are present.

As used herein, the term “heteroalkynyl” refers to alkynyl groups in which one or more triply bonded carbon atoms are replaced by a heteroatom. Heteroalkynyl groups can be linear, branched, or cyclic. Preferred heteroalkynyl groups have two to 30 carbon atoms, i.e., C₂-C₃₀ heteroalkynyl. In some forms, a C₂-C₃₀ heteroalkynyl can be a linear C₂-C₃₀ heteroalkynyl, a branched C₂-C₃₀ heteroalkynyl, a cyclic C₂-C₃₀ heteroalkynyl, a linear or branched C₂-C₃₀ heteroalkynyl, a linear or cyclic C₂-C₃₀ heteroalkynyl, a branched or cyclic C₂-C₃₀ heteroalkynyl, or a linear, branched, or cyclic C₂-C₃₀ heteroalkynyl. More preferred heteroalkynyl groups have two to 20 carbon atoms, i.e., C₂-C₂₀ heteroalkynyl. In some forms, a C₂-C₂₀ heteroalkynyl can be a linear C₂-C₂₀ heteroalkynyl, a branched C₂-C₂₀ heteroalkynyl, a cyclic C₂-C₂₀ heteroalkynyl, a linear or branched C₂-C₂₀ heteroalkynyl, a branched or cyclic C₂-C₂₀ heteroalkynyl, or a linear, branched, or cyclic C₂-C₂₀ heteroalkynyl. Still more preferred heteroalkynyl groups have two to 10 carbon atoms, i.e., C₂-C₁₀ heteroalkynyl. In some forms, a C₂-C₁₀ heteroalkynyl can be a linear C₂-C₁₀ heteroalkynyl, a branched C₂-C₁₀ heteroalkynyl, a cyclic C₂-C₁₀ heteroalkynyl, a linear or branched C₂-C₁₀ heteroalkynyl, a branched or cyclic C₂-C₁₀ heteroalkynyl, or a linear, branched, or cyclic C₂-C₂₀ heteroalkynyl. The most preferred heteroalkynyl groups have two to 6 carbon atoms, i.e., C₂-C₆ heteroalkynyl. In some forms, a C₂-C₆ heteroalkynyl can be a linear C₂-C₆ heteroalkynyl, a branched C₂-C₆ heteroalkynyl, a cyclic C₂-C₆ heteroalkynyl, a linear or branched C₂-C₆ heteroalkynyl, a branched or cyclic C₂-C₆ heteroalkynyl, or a linear, branched, or cyclic C₂-C₆ heteroalkynyl. Preferred C₂-C₆ heteroalkynyl groups have two to four carbons, i.e., C₂-C₄ heteroalkynyl. In some forms, a C₂-C₄ heteroalkynyl can be a linear C₂-C₄ heteroalkynyl, a branched C₂-C₄ heteroalkynyl, a cyclic C₂-C₄ heteroalkynyl, a linear or branched C₂-C₄ heteroalkynyl, a branched or cyclic C₂-C₄ heteroalkynyl, or a linear, branched, or cyclic C₂-C₄ heteroalkynyl. If the heteroalkynyl is branched, it is understood that at least four carbons are present. If heteroalkynyl is cyclic, it is understood that at least three carbons are present.

As used herein, the term “aryl” refers to univalent groups derived from arenes by removal of a hydrogen atom from a ring atom. Arenes are monocyclic and polycyclic aromatic hydrocarbons. In polycyclic aryl groups, the rings can be attached together in a pendant manner or can be fused. Preferred aryl groups have six to 50 carbon atoms, i.e., C₆-C₅₀ aryl. In some forms, a C₆-C₅₀ aryl can be a branched C₆-C₅₀ aryl, a monocyclic C₆-C₅₀ aryl, a polycyclic C₆-C₅₀ aryl, a branched polycyclic C₆-C₅₀ aryl, a fused polycyclic C₆-C₅₀ aryl, or a branched fused polycyclic C₆-C₅₀ aryl. More preferred aryl groups have six to 30 carbon atoms, i.e., C₆-C₃₀ aryl. In some forms, a C₆-C₃₀ aryl can be a branched C₆-C₃₀ aryl, a monocyclic C₆-C₃₀ aryl, a polycyclic C₆-C₃₀ aryl, a branched polycyclic C₆-C₃₀ aryl, a fused polycyclic C₆-C₃₀ aryl, or a branched fused polycyclic C₆-C₃₀ aryl. Even more preferred aryl groups have six to 20 carbon atoms, i.e., C₆-C₂₀ aryl. In some forms, a C₆-C₂₀ aryl can be a branched C₆-C₂₀ aryl, a monocyclic C₆-C₂₀ aryl, a polycyclic C₆-C₂₀ aryl, a branched polycyclic C₆-C₂₀ aryl, a fused polycyclic C₆-C₂₉ aryl, or a branched fused polycyclic C₆-C₂₉ aryl. The most preferred aryl groups have six to twelve carbon atoms, i.e., C₆-C₁₂ aryl. In some forms, a C₆-C₁₂ aryl can be a branched C₆-C₁₂ aryl, a monocyclic C₆-C₁₂ aryl, a polycyclic C₆-C₁₂ aryl, a branched polycyclic C₆-C₁₂ aryl, a fused polycyclic C₆-C₁₂ aryl, or a branched fused polycyclic C₆-C₁₂ aryl. Preferred C₆-C₁₂ aryl groups have six to eleven carbon atoms, i.e., C₆-C₁₁ aryl. In some forms, a C₆-C₁₁ aryl can be a branched C₆-C₁₁ aryl, a monocyclic C₆-C₁₁ aryl, a polycyclic C₆-C₁₁ aryl, a branched polycyclic C₆-C₁₁ aryl, a fused polycyclic C₆-C₁₁ aryl, or a branched fused polycyclic C₆-C₁₁ aryl. More preferred C₆-C₁₂ aryl groups have six to nine carbon atoms, i.e., C₆-C₉ aryl. In some forms, a C₆-C₉ aryl can be a branched C₆-C₉ aryl, a monocyclic C₆-C₉ aryl, a polycyclic C₆-C₉ aryl, a branched polycyclic C₆-C₉ aryl, a fused polycyclic C₆-C₉ aryl, or a branched fused polycyclic C₆-C₉ aryl. The most preferred C₆-C₁₂ aryl groups have six carbon atoms, i.e., C₆ aryl. In some forms, a C₆ aryl can be a branched C₆ aryl or a monocyclic C₆ aryl.

As used herein, the term “heteroaryl” refers to univalent groups derived from heteroarenes by removal of a hydrogen atom from a ring atom. Heteroarenes are heterocyclic compounds derived from arenes by replacement of one or more methine (—C═) and/or vinylene (—CH═CH—) groups by trivalent or divalent heteroatoms, respectively, in such a way as to maintain the continuous i-electron system characteristic of aromatic systems and a number of out-of-plane i-electrons corresponding to the Hückel rule (4n+2). In polycyclic heteroaryl groups, the rings can be attached together in a pendant manner or can be fused. Preferred heteroaryl groups have three to 50 carbon atoms, i.e., C₃-C₅₀ heteroaryl. In some forms, a C₃-C₅₀ heteroaryl can be a branched C₃-C₅₀ heteroaryl, a monocyclic C₃-C₅₀ heteroaryl, a polycyclic C₃-C₅₀ heteroaryl, a branched polycyclic C₃-C₅₀ heteroaryl, a fused polycyclic C₃-C₅₀ heteroaryl, or a branched fused polycyclic C₃-C₅₀ heteroaryl. More preferred heteroaryl groups have six to 30 carbon atoms, i.e., C₆-C₃₀ heteroaryl. In some forms, a C₆-C₃₀ heteroaryl can be a branched C₆-C₃₀ heteroaryl, a monocyclic C₆-C₃₀ heteroaryl, a polycyclic C₆-C₃₀ heteroaryl, a branched polycyclic C₆-C₃₀ heteroaryl, a fused polycyclic C₆-C₃₀ heteroaryl, or a branched fused polycyclic C₆-C₃₉ heteroaryl. Even more preferred heteroaryl groups have six to 20 carbon atoms, i.e., C₆-C₂₀ heteroaryl. In some forms, a C₆-C₂₀ heteroaryl can be a branched C₆-C₂₀ heteroaryl, a monocyclic C₆-C₂₀ heteroaryl, a polycyclic C₆-C₂₀ heteroaryl, a branched polycyclic C₆-C₂₀ heteroaryl, a fused polycyclic C₆-C₂₀ heteroaryl, or a branched fused polycyclic C₆-C₂₀ heteroaryl. The most preferred heteroaryl groups have six to twelve carbon atoms, i.e., C₆-C₁₂ heteroaryl. In some forms, a C₆-C₁₂ heteroaryl can be a branched C₆-C₁₂ heteroaryl, a monocyclic C₆-C₁₂ heteroaryl, a polycyclic C₆-C₁₂ heteroaryl, a branched polycyclic C₆-C₁₂ heteroaryl, a fused polycyclic C₆-C₁₂ heteroaryl, or a branched fused polycyclic C₆-C₁₂ heteroaryl. Preferred C₆-C₁₂ heteroaryl groups have six to eleven carbon atoms, i.e., C₆-C₁₁ heteroaryl. In some forms, a C₆-C₁₁ heteroaryl can be a branched C₆-C₁₁ heteroaryl, a monocyclic C₆-C₁₁ heteroaryl, a polycyclic C₆-C₁₁ heteroaryl, a branched polycyclic C₆-C₁₁ heteroaryl, a fused polycyclic C₆-C₁₁ heteroaryl, or a branched fused polycyclic C₆-C₁₁ heteroaryl. More preferred C₆-C₁₂ heteroaryl groups have six to nine carbon atoms, i.e., C₆-C₉ heteroaryl. In some forms, a C₆-C₉ heteroaryl can be a branched C₆-C₉ heteroaryl, a monocyclic C₆-C₉ heteroaryl, a polycyclic C₆-C₉ heteroaryl, a branched polycyclic C₆-C₉ heteroaryl, a fused polycyclic C₆-C₉ heteroaryl, or a branched fused polycyclic C₆-C₉ heteroaryl. The most preferred C₆-C₁₂ heteroaryl groups have six carbon atoms, i.e., C₆ heteroaryl. In some forms, a C₆ heteroaryl can be a branched C₆ heteroaryl, a monocyclic C₆ heteroaryl, a polycyclic C₆ heteroaryl, a branched polycyclic C₆ heteroaryl, a fused polycyclic C₆ heteroaryl, or a branched fused polycyclic C₆ heteroaryl.

As used herein, the term “thiolate” refers to any derivatives of thiols, in which a metal or other cation replaces the hydrogen attached to the sulfur.

As used herein, the term “phosphine” refers to organophosphorus compounds with the formula R₃P, R₄P₂, or R₅P₃, where R represents an organic substituent.

As used herein, the term “amine” refers to compounds and functional groups that contain a basic nitrogen atom with a lone pair. Primary amines arise when one of three hydrogen atoms in ammonia is substituted by an organic substituent. Secondary amines have two organic substituents bound to the nitrogen together with one hydrogen. In tertiary amine, nitrogen has three organic substituents.

As used herein, the term “signal/noise ratio” refers to the level of a desired signal to the level of background noise.

As used herein, the term “conductive substrate” refers to a substance capable of conducting an electric current.

As used herein, the term “near-IR” refers to the region of the electromagnetic spectrum from about 650 nm to about 1500 nm.

As used herein, the term “room temperature” refers to about 293 K, under atmospheric pressure.

As used herein, the term “same conditions” refers to both environmental conditions and experimental conditions for performing a reaction. Environmental conditions include temperature, pressure, and solvent. Experimental conditions include sample, setup, and optimal parameters under which a reaction progresses optimally.

As used herein, the term “linear sweeping” refers to a method where the applied potential is swept linearly in time from one value to another value.

As used herein, the term “cyclic sweeping” refers to a method where the applied potential is swept linearly in time from one value to another value and then swept linearly in time to return to the initial value.

As used herein, the term “stepping” refers to a method where the applied potential is instantaneously jumped from one value to another value. “Instantaneously” refers to the fastest time a step could be applied and detected, which depends on the time constant of a measurement system (e.g. solution, electrodes, etc.). The time constant of the measurement system can be seconds, milliseconds, or microseconds. Thus, for example, if the results of the step can be applied and detected to a resolution of a second, then instantaneously refers to the results being detected within two seconds of the start or reference time.

As used herein, the term “piperazine” refers to an organic compound that consists of a six-membered ring containing two nitrogen atoms at opposite positions in the ring.

Use of the term “about” is intended to describe values either above or below the stated value in a range of approx. +/−10%; in other instances the values can range in value either above or below the stated value in a range of approx. +/−5%; in other instances the values can range in value either above or below the stated value in a range of approx. +/−2%; in other instances the values can range in value either above or below the stated value in a range of approx. +/−1%. The preceding ranges are intended to be made clear by context, and no further limitation is implied.

Numerical ranges disclosed in the present application of any type, disclose individually each possible number that such a range could reasonably 5 encompass, as well as any sub-ranges and combinations of sub-ranges encompassed therein.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed.

No language in the specification should be construed as indicating any non-claimed element as essential to the practice of the invention.

### II. Electrochemiluminescence Sensors

ECL sensors containing metal nanoclusters that are capable of detecting analytes in a sample have been discovered to produce high ECL signal output and high signal/noise ratio. In some instances, the ECL is in near-IR range. In some instances, the ECL is in a range between about 700 nm and about 1000 nm. In some instances, the ECL is in a range between about 650 nm and about 1000 nm. In some instances, the ECL signal is higher than tris(bipyridine)ruthenium(II) complex (Rubpy) under the same conditions. In some instances, the ECL is at least 2 times higher than Rubpy under the same conditions (Rubpy has been the ECL standard since its establishment). In some instances, the ECL is at least 5 times higher than Rubpy under the same conditions. In some instances, the ECL is at least 10 times higher than Rubpy under the same conditions. In some instances, the ECL is at least 20 times higher than Rubpy under the same conditions. In some instances, In some instances, the ECL is at least 50 times higher than Rubpy under the same conditions. In some instances, the ECL is at least 100 times higher than Rubpy under the same conditions. In some instances, the ECL is at least 120 times higher than Rubpy under the same conditions. In some instances, the ECL is at least 150 times higher than Rubpy under the same conditions. In some instances, the ECL is at least 200 times higher than Rubpy under the same conditions. In some instances, the ECL is at least 250 times higher than Rubpy under the same conditions. In some instances, the ECL is at least 300 times higher than Rubpy under the same conditions. In some instances, the ECL is at least 350 times higher than Rubpy under the same conditions. In some instances, the ECL is at least 400 times higher than Rubpy under the same conditions.

In some instances, the metal nanoclusters are capable of generating ECL in the absence of coreactants (i.e. self-annihilation ECL). In an exemplary case, the self-annihilation ECL intensity from AgₓAu₂₅₋ₓ nanoclusters (x is a positive integer ≤13) (e.g., Au₁₂Ag₁₃ nanoclusters) is about ten times higher than that from Ru(bpy)₃ under the same conditions. In some instances, the metal nanoclusters are capable of generating ECL in the presence of a coreactant (i.e. coreactant ECL). In an exemplary case, with a coreactant such as tripropylamine (TPrA), the coreactant ECL of AgₓAu₂₅₋ₓ nanoclusters (x is a positive integer ≤13) (e.g., Au₁₂Ag₁₃ nanoclusters) is about 400 times higher than Ru(bpy)₃ under the same conditions.

In some instances, the strong ECL of the metal nanoclusters can be attributed to one or more metal atoms in the metal core of the metal nanoclusters that produce stability of the metal core. For example, it has been discovered that the strong ECL of Au₁₂Ag₁₃ nanoclusters can be attributed to the 13th Ag atom at the central position. Without being bound to a particular theory of operation, this central Ag atom appears to stabilize the charges on LUMO orbital and makes the rod-shape Ag₁₃Au₁₂ core more rigid. Thus, arrangements of metal atoms that produce similar stability can also produce higher ECL. Such metal nanoclusters with high ECL provide new tools in applications such as sensing and assay analysis.

The metal nanoclusters can be organo-soluble or aqueous soluble. In some instances, the metal nanoclusters are organo-soluble. In some instances, the metal nanoclusters are aqueous soluble. Two or more ECL sensors can be used together as an ECL sensing array. The ECL sensors in the sensing array can contain the same or different metal nanoclusters. In some instances, each of the ECL sensors in the sensing array contains the same metal nanoclusters. In some instances, the ECL sensors in the sensing array contain different metal nanoclusters, such as two or more different metal nanoclusters. For example, two or more of the ECL sensors contain a first metal nanocluster and one or more ECL sensors contain a second metal nanocluster that is different from the first metal nanocluster.

In some instances, the ECL sensors can include a conductive substrate. In some instances, the metal nanoclusters can be assembled on the surface of the conductive substrate. In some instances, the metal nanoclusters are not assembled on the surface of the conductive substrate but can reach a close proximity to the surface of the conductive substrate to allow electron transfers between the metal nanoclusters and the conductive substrate. In some instances, the metal nanoclusters are organo-soluble metal nanoclusters and assembled on the surface of the conductive substrate. In some instances, the metal nanoclusters are aqueous soluble and assembled on the surface of the conductive substrate. In some instances, the metal nanoclusters are aqueous soluble metal nanoclusters and not assembled on the surface of the conductive substrate but can reach a close proximity to the surface of the conductive substrate to allow electron transfers between the metal nanoclusters and the conductive substrate. In some instances, the metal nanoclusters are organo-soluble metal nanoclusters and not assembled on the surface of the conductive substrate but can reach a close proximity to the surface of the conductive substrate to allow electron transfers between the metal nanoclusters and the conductive substrate.

In instances where metal nanoclusters are assembled on the surface of the conductive substrate, a high surface coverage and uniform metal nanocluster film on the conductive substrate surface is desired to generate strong and consistent signals for detection applications. In some instances, organo-soluble metal nanoclusters are assembled on the conductive substrate surface such that the formed metal nanocluster film can remain on the surface of the conductive substrate in an aqueous environment, such as an aqueous buffer solution (e.g. phosphate buffered solution). Typically, ECL generation from a metal nanocluster film assembled on the conductive substrate surface can eliminate the diffusion process involving metal nanoclusters, thereby simply the ECL generation mechanism and enhance the ECL signal and/or current signals of the metal nanoclusters.

In some instances, the ECL sensors can include coreactants. In some instances, the coreactants can be assembled on the surface of the conductive substrate together with the metal nanoclusters. In some instances, the coreactants can be assembled on the surface of the conductive substrate without the metal nanoclusters on the surface of the conductive substrate. In some instances, the coreactants are not assembled on the surface of the conductive substrate but can reach a close proximity to the surface of the conductive substrate to allow electron transfers between the coreactants and the conductive substrate. In some instances, the coreactants are not assembled on the surface of the conductive substrate and the metal nanoclusters are assembled on the surface of the conductive substrate. In some instances, both the coreactants and the metal nanoclusters are not assembled on the surface of the conductive substrate but can reach a close proximity to the surface of the conductive substrate to allow electron transfers between the coreactants and the conductive substrate and/or between the metal nanoclusters and the conductive substrate. The coreactants can be associated with the metal nanoclusters covalently or non-covalently. In some instances, the coreactants are covalently bound to the metal nanoclusters. In some instances, the coreactant can associate with the metal nanoclusters through non-covalent interactions such as electrostatic, π effects, van der Waals forces, hydrogen bonding, and combinations thereof. In some instances, the coreactants are not associated with the nanoclusters but can reach a close proximity to the metal nanoclusters to allow electron transfer between the coreactants and the metal nanoclusters.

In some instances, the ECL sensors include organo-soluble metal nanoclusters and do not include coreactants. In some instances, the ECL sensors include organo-soluble metal nanoclusters and coreactants. In some instances, the ECL sensors include organo-soluble metal nanoclusters and coreactants attached on the nanoclusters covalently or non-covalently. In some instances, the ECL sensors include organo-soluble metal nanoclusters and coreactants attached on the nanoclusters covalently. In some instances, the ECL sensors include organo-soluble metal nanoclusters and coreactants attached on the nanoclusters non-covalently. In some instances, the ECL sensors include organo-soluble metal nanoclusters assembled on the surface of the conductive substrate and does not include coreactants. In some instances, the ECL sensors include coreactants and organo-soluble metal nanoclusters assembled on the surface of the conductive substrate.

In some instances, the ECL sensors include aqueous soluble metal nanoclusters and do not include coreactants. In some instances, the ECL sensors include aqueous soluble metal nanoclusters and coreactants. In some instances, the ECL sensors include aqueous soluble metal nanoclusters and coreactants attached on the nanoclusters covalently or non-covalently. In some instances, the ECL sensors include aqueous soluble metal nanoclusters and coreactants attached on the nanoclusters covalently. In some instances, the ECL sensors include aqueous soluble metal nanoclusters and coreactants attached on the nanoclusters non-covalently. In some instances, the ECL sensors include aqueous soluble metal nanoclusters assembled on the surface of the conductive substrate and does not include coreactants. In some instances, the ECL sensors include coreactants and aqueous soluble metal nanoclusters assembled on the surface of the conductive substrate. In some instances, the ECL sensors include aqueous soluble metal nanoclusters assembled on the surface of the conductive substrate and coreactants attached on the nanoclusters covalently or non-covalently. In some instances, the ECL sensors include aqueous soluble metal nanoclusters assembled on the surface of the conductive substrate and coreactants attached on the nanoclusters covalently. In some instances, the ECL sensors include aqueous soluble metal nanoclusters assembled on the surface of the conductive substrate and coreactants attached on the nanoclusters non-covalently.

The ECL sensors and ECL sensing arrays can be attached to a reader containing an acquisition system and/or a display component to form an ECL sensing system. The sensing system can be portable, and the acquisition system and/or a display component can be attached or disconnected from the ECL sensors and ECL sensing arrays as needed. The ECL sensors and ECL sensing arrays can be disposable. In some instances, the ECL sensors and ECL sensing arrays can be disposed after a single use or multiple uses. In some instances, the ECL sensors and ECL sensing array are disposed after a single use. In some instances, the ECL sensors and ECL sensing array can be reused for at least one time, at least 2 times, at least 4 times, at least 5 times, or at least 10 times before disposal.

A. Metal Nanoclusters

The metal nanoclusters typically include a core and a plurality of ligands. The core can contain metal atoms of the same metal or a mixture of metal atoms of more than one metal. In some instances, the core contains metal atoms of the same metal. In some instances, the core contains a mixture of different types of metal atoms. The ligands can be bound to the core covalently or semi-covalently. The ligands can be in the form of a monolayer or multilayers. In some instances, the ligands are in the form of a monolayer. In some instances, the metal nanoclusters further include targeting moieties bound to the core and/or the ligands of the metal nanoclusters. In some instances, the metal nanoclusters further include targeting moieties bound to the ligands of the metal nanoclusters. The ligands can be thiolates, phosphines, other non-metallic elements, or combinations thereof. In some instances, the ligands bound to the core are thiolates. In some instances, the ligands bound to the nanoclusters are phosphines. In some instances, the ligands bound to the nanoclusters are a mixture of thiolates and phosphines. In some instances, the ligands bound to the nanoclusters are a mixture of thiolates and other non-metallic elements. In some instances, the ligands bound to the nanoclusters are a mixture of phosphines and other non-metallic elements. In some instances, the ligands bound to the nanoclusters are a mixture of thiolates, phosphines, and other non-metallic elements. The non-metallic elements in the nanoclusters can be bound to the core and/or the thiolates and/or phosphines of the metal nanoclusters. In some instances, the non-metallic elements are bound to the core of the metal nanoclusters. In some instances, the non-metallic elements are bound to the thiolates and/or phosphines of the metal nanoclusters. In some instances, the non-metallic elements are bound to the core and the thiolates and/or phosphines of the metal nanoclusters. In some instances, the non-metallic elements can be oxygen, sulfur, selenium, phosphorous, halogens, or combinations thereof.

In some instances, the ligands can be thiolates, phosphines, halogens, or combinations thereof. In some instances, the ligands are thiolates. In some instances, the ligands are phosphines. In some instances, the ligands of the metal nanoclusters are halogens. In some instances, the ligands of the metal nanoclusters can are a mixture of thiolates and halogens. In some instances, the ligands of the metal nanoclusters are a mixture of phosphines and halogens. In some instances, the ligands are a mixture of thiolates and phosphines. In some instances, the ligands of the metal nanoclusters can contain a mixture of thiolates, phosphines, and halogens. The metal nanoclusters can further include targeting moieties for reaching desired site(s) in vivo or for capturing an analyte or a target containing and/or producing the analyte in vivo and in vitro. In some instances, the metal nanoclusters can include coreactants bound to the core and/or the ligands covalently or non-covalently. In some instances, the coreactants are covalently bound to the ligands. In some instances, the coreactants are bound to the ligands though non-covalent interactions such as electrostatic, π effects, van der Waals forces, hydrogen bonding, and combinations thereof.

The metal nanoclusters can have any suitable shapes (e.g., regular shapes such as rod-shaped, spherical, oval, cylindrical, cubical, and irregular shapes). In some instances, the metal nanoclusters can have regular shapes including, but not limited to, rod-shaped, spherical, oval, cylindrical, and cubical. In some instances, the metal nanoclusters are rod-shaped or spherical. In some instances, the metal nanoclusters are spherical. In some instances, the metal nanoclusters are rod-shaped.

The metal nanoclusters can be organo-soluble or aqueous soluble. In some instances, the metal nanoclusters are organo-soluble. In some instances, the metal nanoclusters are aqueous soluble. The solubility of the metal nanoclusters generally depends on the polarity and/or hydrophilicity of the ligands. In some instances, the solubility of the metal nanoclusters can be tuned by functionalizing the ligands with hydrophilic or hydrophobic groups. In some instances, the metal nanoclusters are tuned to be organo-soluble by functionalizing the ligands with hydrophobic groups. In some instances, the metal nanoclusters are tuned to be aqueous soluble by functionalizing the ligands with hydrophilic groups. In some instances, the functionalization can be performed before ligand-attachment to the core or after ligand-attachment to the core of the metal nanoclusters.

The energetics of metal nanoclusters generally depend on their specific compositions and structures. Their electronic properties can be tuned from molecular-liked behavior, such as HOMO-LUMO (highest occupied and lowest unoccupied molecular orbital) transitions (Murray, Chem. Rev., 108(7):2688-2720 (2008)), to semiconductor or metallic-like quantized single-electron charging (Wang, et al., ACS Nano, 9(8):8344-8351 (2015)).

Metal nanoclusters described herein show high ECL signal. The ECL signal can be in the near-IR range or visible range. In some instances, the ECL signal is in the near-IR range. In some instances, the ECL signal is in a range between about 650 nm and about 1500 nm. In some instances, the ECL signal is in a range between about 650 nm and about 1000 nm. In some instances, the ECL signal is in a range between about 700 nm and about 1000 nm. In some instances, the ECL signal is in a range between about 700 nm and about 1500 nm. In some instances, the ECL signal is in the visible range. In some instances, the metal nanoclusters show ECL signal higher than Rubpy under the same conditions. In some instances, the ECL signal is at least 2 times higher than Rubpy under the same conditions. In some instances, the ECL is at least 5 times higher than Rubpy under the same conditions. In some instances, the ECL is at least 10 times higher than Rubpy under the same conditions. In some instances, the ECL is at least 20 times higher than Rubpy under the same conditions. In some instances, In some instances, the ECL is at least 50 times higher than Rubpy under the same conditions. In some instances, the ECL is at least 100 times higher than Rubpy under the same conditions. In some instances, the ECL is at least 120 times higher than Rubpy under the same conditions. In some instances, the ECL is at least 150 times higher than Rubpy under the same conditions. In some instances, the ECL is at least 200 times higher than Rubpy under the same conditions. In some instances, the ECL is at least 250 times higher than Rubpy under the same conditions. In some instances, the ECL is at least 300 times higher than Rubpy under the same conditions. In some instances, the ECL is at least 350 times higher than Rubpy under the same conditions. In some instances, the ECL is at least 400 times higher than Rubpy under the same conditions.

In some instances, the metal nanoclusters are organo-soluble gold (Au) nanoclusters. In some instances, the metal nanoclusters are organo-soluble Au nanoclusters protected by a monolayer of monothiolates. In some instances, the metal nanoclusters are organo-soluble Au nanoclusters protected by a monolayer of ligands selected from 4-tert-butylbenzyl mercaptan (TBBM), 4-tert-butylbenzenethiol (TBBT), phenylethylenethiol (PET), naphthalenethiol (NT), and combinations thereof.

In some instances, the metal nanoclusters are organo-soluble gold/silver (AuAg) nanoclusters. In some instances, the metal nanoclusters are organo-soluble AuAg nanoclusters protected by a monolayer of thiolates, phosphines, halogens, or combinations thereof. In some instances, the metal nanoclusters are organo-soluble AuAg nanoclusters protected by a monolayer of a mixture of thiolates and phosphines. In some instances, the metal nanoclusters are organo-soluble AuAg nanoclusters protected by a monolayer of thiolates. In some instances, the metal nanoclusters are organo-soluble AuAg nanoclusters protected by a monolayer of monothiolates. In some instances, the metal nanoclusters are organo-soluble AuAg nanoclusters protected by a monolayer of phosphines. In some instances, the metal nanoclusters are organo-soluble AuAg nanoclusters protected by a monolayer of a mixture of thiolates and phosphines and include one or more non-metallic elements bound to the Au and/or Ag atoms of the core. In some instances, the metal nanoclusters are organo-soluble AuAg nanoclusters protected by a monolayer of a mixture of thiolates, phosphines, and halogens, where the halogens are bound to the Au and/or Ag atoms of the core. In some instances, the metal nanoclusters are organo-soluble AuAg nanoclusters protected by a monolayer of a mixture of thiolates, phosphines, and chlorines, where the chlorines are bound to the Au and/or Ag atoms of the core. In some instances, the metal nanoclusters are organo-soluble AuAg nanoclusters protected by a monolayer of a mixture of thiolates, phosphines, and chlorines, where the chlorines are bound to the Ag atoms of the core. In some instances, the metal nanoclusters are organo-soluble AuAg nanoclusters protected by a monolayer of a mixture of sulfydryl, triphenylphosphine, and chorines, where the chlorines are bound to the Ag atoms of the core. In some instances, the number of Au and Ag atoms in the organo-soluble AuAg nanoclusters has an Au:Ag ratio of (25-x):x, x is a positive integer ≤13. In some instances, the number of Au and Ag atoms in the organo-soluble AuAg nanoclusters has an Au:Ag ratio of 12:13. In some instances, the number of thiolates and phosphines in the organo-soluble AuAg nanoclusters has a thiolates:phosphines ratio of 1:2. In some instances, the organo-soluble AuAg nanoclusters have an Au:Ag ratio of 12:13 and a thiolates:phosphines ratio of 1:2. In some instances, the organo-soluble AuAg nanoclusters have a core containing 25 metal atoms. In some instances, the organo-soluble AuAg nanoclusters have 12 Au atoms and 13 Ag atoms.

1. Core

The core can contain metal atoms of the same metal (also referred therein as “same type,” i.e., with no metal atoms of other metals present) or a mixture of metal atoms of different types. In some instances, the core contains metal atoms of the same type. In some instances, the core contains a mixture of metal atoms of different types. For example, the core contains two or more metal atoms of a first metal and one or more metal atoms of a second metal that is different from the first metal. In some instances, the metal atoms can be transitional metals, Group I metals, Group II metals, Group XIII metals, or combinations thereof. Exemplary metals for the metal atoms in the core include, but are not limited to, gold, silver, aluminum, tin, magnesium, copper, nickel, iron, cobalt, magnesium, platinum, palladium, iridium, vanadium, rhodium, and ruthenium. In some instances, the metal atoms are gold metal atoms. In some instances, a mixture of metal atoms can contain gold metal atoms and silver metal atoms.

The core can have a largest dimension less than about 10 nm, less than about 9 nm, less than about 8 nm, less than about 7 nm, less than about 6 nm, less than about 5 nm, less than about 4 nm, less than about 3 nm, less than about 2.5 nm, less than about 2.2 nm, less than about 2 nm, less than about 1.5 nm, or less than about 1 nm. Preferably, the core can have a largest dimension less than about 2.2 nm. The size of the core can affect the physical properties of the metal nanocluster such as electronic, magnetic, and/or optical properties of the metal nanocluster.

The total number of metal atoms in the core can affect the size of the core. The core can contain between 3 and about 1000 metal atoms. In some instances, the core can contain between 10 and 200 metal atoms, between 10 and 150 metal atoms, between 10 and 133 metal atoms, between 10 and 100 metal atoms, between 10 and 95 metal atoms, between 10 and 90 metal atoms, between 10 and 80 metal atoms, between 10 and 76 metal atoms, between 10 and 70 metal atoms, between 10 and 65 metal atoms, between 10 and 60 metal atoms, between 10 and 55 metal atoms, between 10 and 52 metal atoms, between 10 and 45 metal atoms, between 10 and 40 metal atoms, between 10 and 36 metal atoms, between 10 and 30 metal atoms, between 10 and 28 metal atoms, between 10 and 25 metal atoms, between 10 and 22 metal atoms, or between 10 and 20 metal atoms. In some instances, the core can contain 25 metal atoms. In some instances, the core can contain a mixture of Au atoms and Ag atoms where the total number of Au atoms and Ag atoms is 25. In some instances, the core contains only Au atoms, wherein the number of Au atoms can be 11, 13, 20, 22, 28, 36, 44, 52, 76, 92, 130, or 133. In some instances, the metal nanoclusters are organo-soluble and the core of the metal nanoclusters is not Au₂₅, Au₃₈, or Au₁₄₄. In some instances, the metal nanoclusters are aqueous soluble and the core of the metal nanoclusters is not Au₂₂.

In some instances, the core can contain a mixture of two types of metal atoms where the number of metal atoms of the first type to the number of metal atoms of the second type can have a ratio between about 0.01 and about 25, between about 0.04 and about 25, between about 0.04 and about 24, between about 0.05 and about 20, between about 0.1 and about 15, between about 0.2 and about 10, between about 0.5 and about 5, between about 0.1 and about 1, between about 0.2 and about 1, between about 0.5 and about 1, or between about 0.5 and about 0.9. In some instances, the number of metal atoms of the first type to the number of metal atoms of the second type has a ratio of 12:13. In some instances, the core contains Au atoms and Ag atoms where the number of Au atoms to the number of Ag atoms has a ratio of 12:13. In some instances, the number of metal atoms of the first type to the number of metal atoms of the second type has a ratio of (25-x) to x, where x is a positive integer ≤13. In some instances, the number of Au atoms to the number of metal atoms other than Au atoms has a ratio of (25-x) to x, where x is a positive integer ≤13. In some instances, the number of Au atoms to the number of metal atoms of another type has a ratio of (25-x) to x, where x is a positive integer ≤13. In some instances, the number of gold atoms to the number of silver atoms has a ratio of (25-x) to x, where x is a positive integer ≤13. In some instances, the number of metal atoms of the first type to the number of metal atoms of the second type has a ratio of (38-x) to x, where x is a positive integer ≤13. In some instances, the number of Au atoms to the number of metal atoms other than Au atoms has a ratio of (38-x) to x, where x is a positive integer ≤13. In some instances, the number of Au atoms to the number of metal atoms of another type has a ratio of (38-x) to x, where x is a positive integer ≤13. In some instances, the number of gold atoms to the number of silver atoms has a ratio of (38-x) to x, where x is a positive integer ≤13.

In some instances, the number of metal atoms of the first type to the number of metal atoms of the second type has a ratio of 1:15, 2:15, 3:15, 4:15, 5:15, 6:15, 7:15, 8:15, 9:15, 10:15, 11:15, 12:15, 13:15, 14:15, 15:15, 16:15, 17:15, 18:15, 19:15, 20:15, 21:15, 22:15, 23:15, 24:15, 25:15, 1:14, 2:14, 3:14, 4:14, 5:14, 6:14, 7:14, 8:14, 9:14, 10:14, 11:14, 12:14, 13:14, 14:14, 15:14, 16:14, 17:14, 18:14, 19:14, 20:14, 21:14, 22:14, 23:14, 24:14, 25:14, 1:13, 2:13, 3:13, 4:13, 5:13, 6:13, 7:13, 8:13, 9:13, 10:13, 11:13, 12:13, 13:13, 14:13, 15:13, 16:13, 17:13, 18:13, 19:13, 20:13, 21:13, 22:13, 23:13, 24:13, 25:13, 1:12, 2:12, 3:12, 4:12, 5:12, 6:12, 7:12, 8:12, 9:12, 10:12, 11:12, 12:12, 13:12, 14:12, 15:12, 16:12, 17:12, 18:12, 19:12, 20:12, 21:12, 22:12, 23:12, 24:12, 25:12, 1:11, 2:11, 3:11, 4:11, 5:11, 6:11, 7:11, 8:11, 9:11, 10:11, 11:11, 12:11, 13:11, 14:11, 15:11, 16:11, 17:11, 18:11, 19:11, 20:11, 21:11, 22:11, 23:11, 24:11, 25:11, 1:10, 2:10, 3:10, 4:10, 5:10, 6:10, 7:10, 8:10, 9:10, 10:10, 11:10, 12:10, 13:10, 14:10, 15:10, 16:10, 17:10, 18:10, 19:10, 20:10, 21:10, 22:10, 23:10, 24:10, 25:10, 1:9, 2:9, 3:9, 4:9, 5:9, 6:9, 7:9, 8:9, 9:9, 10:9, 11:9, 12:9, 13:9, 14:9, 15:9, 16:9, 17:9, 18:9, 19:9, 20:9, 21:9, 22:9, 23:9, 24:9, 25:9, 1:8, 2:8, 3:8, 4:8, 5:8, 6:8, 7:8, 8:8, 9:8, 10:8, 11:8, 12:8, 13:8, 14:8, 15:8, 16:8, 17:8, 18:8, 19:8, 20:8, 21:8, 22:8, 23:8, 24:8, 25:8, 1:7, 2:7, 3:7, 4:7, 5:7, 6:7, 7:7, 8:7, 9:7, 10:7, 11:7, 12:7, 13:7, 14:7, 15:7, 16:7, 17:7, 18:7, 19:7, 20:7, 21:7, 22:7, 23:7, 24:7, 25:7, 1:6, 2:6, 3:6, 4:6, 5:6, 6:6, 7:6, 8:6, 9:6, 10:6, 11:6, 12:6, 13:6, 14:6, 15:6, 16:6, 17:6, 18:6, 19:6, 20:6, 21:6, 22:6, 23:6, 24:6, 25:6, 1:5, 2:5, 3:5, 4:5, 5:5, 6:5, 7:5, 8:5, 9:5, 10:5, 11:5, 12:5, 13:5, 14:5, 15:5, 16:5, 17:5, 18:5, 19:5, 20:5, 21:5, 22:5, 23:5, 24:5, 25:5, 1:4, 2:4, 3:4, 4:4, 5:4, 6:4, 7:4, 8:4, 9:4, 10:4, 11:4, 12:4, 13:4, 14:4, 15:4, 16:4, 17:4, 18:4, 19:4, 20:4, 21:4, 22:4, 23:4, 24:4, 25:4, 1:3, 2:3, 3:3, 4:3, 5:3, 6:3, 7:3, 8:3, 9:3, 10:3, 11:3, 12:3, 13:3, 14:3, 15:3, 16:3, 17:3, 18:3, 19:3, 20:3, 21:3, 22:3, 23:3, 24:3, 25:3, 1:2, 2:2, 3:2, 4:2, 5:2, 6:2, 7:2, 8:2, 9:2, 10:2, 11:2, 12:2, 13:2, 14:2, 15:2, 16:2, 17:2, 18:2, 19:2, 20:2, 21:2, 22:2, 23:2, 24:2, 25:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, or 25:1.

In some instances, the number of gold atoms to the number of silver atoms has a ratio of 1:15, 2:15, 3:15, 4:15, 5:15, 6:15, 7:15, 8:15, 9:15, 10:15, 11:15, 12:15, 13:15, 14:15, 15:15, 16:15, 17:15, 18:15, 19:15, 20:15, 21:15, 22:15, 23:15, 24:15, 25:15, 1:14, 2:14, 3:14, 4:14, 5:14, 6:14, 7:14, 8:14, 9:14, 10:14, 11:14, 12:14, 13:14, 14:14, 15:14, 16:14, 17:14, 18:14, 19:14, 20:14, 21:14, 22:14, 23:14, 24:14, 25:14, 1:13, 2:13, 3:13, 4:13, 5:13, 6:13, 7:13, 8:13, 9:13, 10:13, 11:13, 12:13, 13:13, 14:13, 15:13, 16:13, 17:13, 18:13, 19:13, 20:13, 21:13, 22:13, 23:13, 24:13, 25:13, 1:12, 2:12, 3:12, 4:12, 5:12, 6:12, 7:12, 8:12, 9:12, 10:12, 11:12, 12:12, 13:12, 14:12, 15:12, 16:12, 17:12, 18:12, 19:12, 20:12, 21:12, 22:12, 23:12, 24:12, 25:12, 1:11, 2:11, 3:11, 4:11, 5:11, 6:11, 7:11, 8:11, 9:11, 10:11, 11:11, 12:11, 13:11, 14:11, 15:11, 16:11, 17:11, 18:11, 19:11, 20:11, 21:11, 22:11, 23:11, 24:11, 25:11, 1:10, 2:10, 3:10, 4:10, 5:10, 6:10, 7:10, 8:10, 9:10, 10:10, 11:10, 12:10, 13:10, 14:10, 15:10, 16:10, 17:10, 18:10, 19:10, 20:10, 21:10, 22:10, 23:10, 24:10, 25:10, 1:9, 2:9, 3:9, 4:9, 5:9, 6:9, 7:9, 8:9, 9:9, 10:9, 11:9, 12:9, 13:9, 14:9, 15:9, 16:9, 17:9, 18:9, 19:9, 20:9, 21:9, 22:9, 23:9, 24:9, 25:9, 1:8, 2:8, 3:8, 4:8, 5:8, 6:8, 7:8, 8:8, 9:8, 10:8, 11:8, 12:8, 13:8, 14:8, 15:8, 16:8, 17:8, 18:8, 19:8, 20:8, 21:8, 22:8, 23:8, 24:8, 25:8, 1:7, 2:7, 3:7, 4:7, 5:7, 6:7, 7:7, 8:7, 9:7, 10:7, 11:7, 12:7, 13:7, 14:7, 15:7, 16:7, 17:7, 18:7, 19:7, 20:7, 21:7, 22:7, 23:7, 24:7, 25:7, 1:6, 2:6, 3:6, 4:6, 5:6, 6:6, 7:6, 8:6, 9:6, 10:6, 11:6, 12:6, 13:6, 14:6, 15:6, 16:6, 17:6, 18:6, 19:6, 20:6, 21:6, 22:6, 23:6, 24:6, 25:6, 1:5, 2:5, 3:5, 4:5, 5:5, 6:5, 7:5, 8:5, 9:5, 10:5, 11:5, 12:5, 13:5, 14:5, 15:5, 16:5, 17:5, 18:5, 19:5, 20:5, 21:5, 22:5, 23:5, 24:5, 25:5, 1:4, 2:4, 3:4, 4:4, 5:4, 6:4, 7:4, 8:4, 9:4, 10:4, 11:4, 12:4, 13:4, 14:4, 15:4, 16:4, 17:4, 18:4, 19:4, 20:4, 21:4, 22:4, 23:4, 24:4, 25:4, 1:3, 2:3, 3:3, 4:3, 5:3, 6:3, 7:3, 8:3, 9:3, 10:3, 11:3, 12:3, 13:3, 14:3, 15:3, 16:3, 17:3, 18:3, 19:3, 20:3, 21:3, 22:3, 23:3, 24:3, 25:3, 1:2, 2:2, 3:2, 4:2, 5:2, 6:2, 7:2, 8:2, 9:2, 10:2, 11:2, 12:2, 13:2, 14:2, 15:2, 16:2, 17:2, 18:2, 19:2, 20:2, 21:2, 22:2, 23:2, 24:2, 25:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, or 25:1.

In some instances, the core can contain one type of metal atoms (i.e., with no other type of metal atom present), a mixture of two types of metal atoms, a mixture of three types of metal atoms, a mixture of four types of metal atoms, a mixture of five types of metal atoms, or a mixture of six types of metal atoms. The ratio of the different types of metal atoms to the other types of metal atoms in such mixtures can be any ratio, such as the ratios discussed above in the context of mixtures of two types of metal atoms.

2. Ligands

The ligands bound to the core can be the same type of ligands or a mixture of different types of ligands. The ligands can be bound to the core covalently or semi-covalently. The ligands can be in the form of a monolayer or multilayers. In some instances, the ligands are in the form of a monolayer.

The ligands can be thiolates, phosphines, other non-metallic elements, or combinations thereof. In some instances, the ligands are thiolates. In some instances, the ligands are phosphines. In some instances, the ligands of the metal nanoclusters are halogens. In some instances, the ligands of the metal nanoclusters are a mixture of thiolates and halogens. In some instances, the ligands of the metal nanoclusters are a mixture of phosphines and halogens. In some instances, the ligands are a mixture of thiolates and phosphines. In some instances, the ligands of the metal nanoclusters can contain a mixture of thiolates, phosphines, and halogens.

a. Thiolates and Phosphines

The number of thiolates and the number of phosphines bound to the core can have a ratio between about 0.01 and about 100, between about 0.01 and about 50, between about 0.01 and about 20, between about 0.01 and about 10, between about 0.01 and about 5, between about 0.01 and about 2, between about 0.01 and about 1, between about 0.01 and about 0.5, between about 0.1 and about 100, between about 0.1 and about 50, between about 0.1 and about 20, between about 0.1 and about 10, between about 0.1 and about 5, between about 0.1 and about 2, between about 0.1 and about 1, between about 0.1 and about 0.5, between about 0.2 and about 2, or between about 0.2 and about 1. In some instances, the number of thiolates and the number of phosphines has a ratio of about 0.5.

In some instances, the thiolates bound to the core can be monothiolates, dithiolates, or a mixture of both. In some instances, the thiolates bound to the core are monothiolates. In some instances, the thiolates bound to the core are dithiolates. In some instances, the thiolates bound to the core are a mixture of monothiolates and dithiolates. In some instances, the dithiolates bound to the core can be 1,4 dithiolates, 1,2-dithiolates, 1,3-dithiolates, 2,3-dithiolates, 2,4-dithiolates, 3,4-dithiolates, or combinations thereof.

Exemplary thiolates include, but are not limited to, 4-tert-butylbenzyl mercaptan (TBBM), 4-tert-butylbenzenethiol (TBBT), phenylethylenethiol (PET), naphthalenethiol (NT), durene dithiol, SC₂H₄Ph, alkane thiols, p/m/o-benzene thiols, lipoic acid (LA), mercaptosuccinic acid (MSA), tiopronin, methionine, and glutathione.

In some instances, the phosphines bound to the core can be monophosphines, diphosphines, triphosphines, or combinations thereof. In some instances, the phosphines bound to the core are monophosphines. In some instances, the phosphines bound to the core are diphosphines. In some instances, the phosphines bound to the core are triphosphines. In some instances, the phosphines bound to the core are a mixture of monophosphines and diphosphines. In some instances, the phosphines bound to the core are a mixture of monophosphines and triphosphines. In some instances, the phosphines bound to the core are a mixture of diphosphines and triphosphines.

Exemplary phosphines include, but are not limited to, tripheynlphosphine (TPP), TPP derivatives (e.g. substituents (such as hydroxyl group, amino group, etc.) on the phenyl ring of TPP), tri-(fused ring)-phosphine, trioctylphosphine (TOP), and 1,1,1-tris(diphenylphosphinomethyl)ethane (TPPME). In some instances, the ligands can be TBBM. In some instances, the ligands can be TBBT. In some instances, the ligands can be PET. In some instances, the ligands can be NT. In some instances, the ligands can be lipoic acid. In some instances, the ligands can be mercaptosuccinic acid. In some instances, the ligands are not lipoic acid. In some instances, the ligands can be a mixture of triphenylphosphine and PET.

In some instances, the ligands bound to the core are a mixture of monothiolates and monophosphines. In some instances, the ligands bound to the core are a mixture of dithiolates and monophosphines. In some instances, the ligands bound to the core are a mixture of monothiolates and diphosphines. In some instances, the ligands bound to the core are a mixture of dithiolates and diphosphines. In some instances, the ligands bound to the core are a mixture of monothiolates, dithiolates, and monophosphines. In some instances, the ligands bound to the core are a mixture of monothiolates, dithiolates, and diphosphines. In some instances, the ligands bound to the core are a mixture of monothiolates, monophosphines, and diphosphines. In some instances, the ligands bound to the core are a mixture of dithiolates, monophosphines, and diphosphines.

The polarity/hydrophilicity of the ligands can determine the solubility of the metal nanoclusters. Exemplary thiolate ligands for organo-soluble metal nanoclusters include, but are not limited to, 4-tert-butylbenzyl mercaptan (TBBM), 4-tert-butylbenzenethiol (TBBT), phenylethylenethiol (PET), naphthalenethiol (NT), durene dithiol, and SC₂H₄Ph. Exemplary thiolate ligands for aqueous soluble metal nanoclusters include, but are not limited to, lipoic acid (LA), mercaptosuccinic acid (MSA), methionine, tiopronin, and glutathione. An exemplary phosphine ligand for organo-soluble metal nanoclusters is tripheynlphosphine (TPP). In some instances, the metal nanoclusters are aqueous soluble and are not Au₂₂LA₁₂. In some instances, the metal nanocluster are aqueous soluble and are not methionine.

In some instances, organo-soluble metal nanoclusters can be converted to aqueous soluble metal nanocluster by modifying the ligands on the metal nanoclusters with hydrophilic functional groups. Typical functional groups that can make the metal nanoclusters aqueous soluble include charged or uncharged polar groups. Exemplary hydrophilic functional groups include, but are not limited to, hydroxyl groups, carboxylic acid groups, sulfonate groups, sulfate groups, sulfite groups, phosphate groups, phosphonate groups, phosphate groups, amino groups, quaternary ammonium groups, pyridinium groups, nitro groups, oligo-groups, and polyethylene groups. In some instances, the ligands can be functionalized with oligo- or poly-ethylene glycol (PEG) moieties.

The number of ligands bound to the core can vary depending on the number of metal atoms and the arrangement of metal atoms in the core. In some instances, the number of ligands can be between 5 and 5000, between 5 and 1000, between 5 and 500, between 5 and 200, between 5 and 100, between 5 and 90, between 5 and 80, between 5 and 70, between 5 and 60, between 5 and 55, between 5 and 50, between 5 and 45, between 5 and 40, between 5 and 35, between 5 and 30, between 5 and 25, between 5 and 20, between 5 and 15, between 5 and 10, between 10 and 100, between 10 and 90, between 10 and 80, between 10 and 70, between 10 and 60, between 10 and 55, between 10 and 45, between 10 and 40, between 10 and 35, between 10 and 30, between 10 and 25, between 10 and 20, between 10 and 15, between 15 and 100, between 15 and 90, between 15 and 80, between 15 and 70, between 15 and 60, between 15 and 55, between 15 and 45, between 15 and 40, between 15 and 35, between 15 and 30, between 15 and 25, or between 15 and 20. In some instances, the number of ligands bound to the core is 15. In some instances, the number of ligands bound to the core is 16. In some instances, the number of ligands bound to the core is 20. In some instances, the number of ligands bound to the core is 24. In some instances, the number of ligands bound to the core is 28. In some instances, the number of ligands bound to the core is 32. In some instances, the number of ligands bound to the core is 38. In some instances, the number of ligands bound to the core is 44. In some instances, the number of ligands bound to the core is 52. In some instances, the ligands can be 5 thiolates and 10 phosphines. In some instances, the ligands can be 5 PET and 10 triphenylphosphine.

b. Other Non-metallic Elements

The ligands of metal nanoclusters can include other non-metallic elements. The non-metallic elements can be bound to the core and/or other ligands (e.g. thiolates and/or phosphines) of the metal nanoclusters. In some instances, the non-metallic elements are bound to the core of the metal nanoclusters. In some instances, the non-metallic elements are bound to other ligands of the metal nanocluster. In some instances, the non-metallic elements are bound to the core and other ligands of the metal nanoclusters. In some instances, the non-metallic elements are bound to the thiolates and/or phosphines of the metal nanoclusters. In some instances, the non-metallic elements are bound to the metal atoms in the core to bridge the metal atoms. In some instances, the non-metallic elements are bound to the metal atoms on the surface of the core.

Exemplary non-metallic elements include, but are not limited to, oxygen, sulfur, selenium, phosphorous, halogens, and a combination thereof. In some instances, the non-metallic elements included in the metal nanoclusters are halogens including fluorine, chlorine, bromine, iodine, astatine, and a combination thereof. In some instances, the non-metallic elements included in the metal nanoclusters are a combination of different halogens, such as fluorine and chlorine or chlorine and bromine. In some instances, the non-metallic elements included in the metal nanoclusters are chlorine.

The number of non-metallic elements can vary depending on the number of metal atoms in the core, the number of ligands, and the location of binding. In some instances, the number of non-metallic elements included in the metal nanoclusters can be between 1 and 5000, between 1 and 1000, between 1 and 500, between 1 and 200, between 1 and 100, between 1 and 90, between 1 and 80, between 1 and 70, between 1 and 60, between 1 and 55, between 1 and 50, between 1 and 45, between 1 and 40, between 1 and 35, between 1 and 30, between 1 and 25, between 1 and 20, between 1 and 15, between 1 and 10, or between 1 and 5. In some instances, the number of chlorine included in the metal nanoclusters can be between 1 and 5000, between 1 and 1000, between 1 and 500, between 1 and 200, between 1 and 100, between 1 and 90, between 1 and 80, between 1 and 70, between 1 and 60, between 1 and 55, between 1 and 50, between 1 and 45, between 1 and 40, between 1 and 35, between 1 and 30, between 1 and 25, between 1 and 20, between 1 and 15, between 1 and 10, or between 1 and 5.

In some instances, the number of non-metallic elements included in the metal nanoclusters is 2. In some instances, the number of non-metallic elements bound to the core is 2. In some instances, the number of non-metallic elements bound to the metal atoms on the surface of the core is 2. In some instances, the number of chlorine bound to the core is 2. In some instances, 2 chlorines are bound to the Au atoms in the Au core. In some instances, 2 chlorines are bound to the Au atoms in the Au/Ag core. In some instances, 2 chlorines are bound to the Ag atoms in the Au/Ag core.

3. Targeting Moieties

The metal nanoclusters can include targeting moieties to provide recognition and/or targeting functions. For example, the targeting moieties of the metal nanoclusters can be used to capture an analyte or a target containing and/or producing the analyte. Targets can be captured include, but are not limited to cells, bacteria, proteins, enzymes, nucleic acids, metabolites, and drugs. The metal nanoclusters can be functionalized with one or more targeting moieties. The targeting moieties can be bound to the core of the metal nanoclusters and/or the ligands of the metal nanoclusters. In some instances, the targeting moieties can be bound to the core of the metal nanoclusters. In some instances, the targeting moieties can be bound to the ligands of the metal nanoclusters. In some instances, the targeting moieties can be bound to the core and the ligands of the metal nanoclusters. The bounding between the targeting moieties and the metal nanoclusters can be covalent and/or non-covalent. In some instances, the targeting moieties are bound to the core covalently and/or non-covalently. In some instances, the targeting moieties are bound to the core covalently. In some instances, the targeting moieties are bound to the core non-covalently. In some instances, the targeting moieties are bound to the core both covalently and non-covalently. In some instances, the targeting moieties are bound to the ligands covalently and/or non-covalently. In some instances, the targeting moieties are bound to the ligands covalently. In some instances, the targeting moieties are bound to the ligands non-covalently. In some instances, the targeting moieties are bound to the ligands both covalently and non-covalently.

Targeting moieties are known in the art. Exemplary targeting moieties include, but are not limited to, oligo or polynucleotides, antibodies, receptors, enzymes, proteins, oligonucleic acids, biomarkers, aptamers, folic acid, cofactors, biotin, lactoferrin, transferrin, tat protein, and streptavidin. In some instances, the targeting moieties are aptamers, antibodies, or a combination thereof. In some instances, the targeting moieties are aptamers. In some instances, the targeting moieties are antibodies. In some instances, the targeting moieties are a combination of aptamers and antibodies.

In some instances, the metal nanoclusters include aptamers and/or antibodies bound to the ligands of the metal nanoclusters covalently and/or non-covalently. In some instances, the metal nanoclusters include aptamers bound to the ligands of the metal nanoclusters covalently and/or non-covalently. In some instances, the metal nanoclusters include aptamers bound to the ligands of the metal nanoclusters covalently. In some instances, the metal nanoclusters include aptamers bound to the ligands of the metal nanoclusters non-covalently. In some instances, the metal nanoclusters include aptamers bound to the ligands of the metal nanoclusters both covalently and non-covalently. In some instances, the metal nanoclusters include antibodies bound to the ligands of the metal nanoclusters covalently and/or non-covalently. In some instances, the metal nanoclusters include antibodies bound to the ligands of the metal nanoclusters covalently. In some instances, the metal nanoclusters include antibodies bound to the ligands of the metal nanoclusters non-covalently. In some instances, the metal nanoclusters include antibodies bound to the ligands of the metal nanoclusters both covalently and non-covalently. In some instances, the metal nanoclusters include aptamers and antibodies bound to the ligands of the metal nanoclusters covalently and/or non-covalently. In some instances, the metal nanoclusters include aptamers and antibodies bound to the ligands of the metal nanoclusters covalently. In some instances, the metal nanoclusters include aptamers and antibodies bound to the ligands of the metal nanoclusters non-covalently. In some instances, the metal nanoclusters include aptamers and antibodies bound to the ligands of the metal nanoclusters both covalently and non-covalently.

B. Conductive Substrate

The conductive substrate is a substance capable of conducting an electric current. In some instances, the metal nanoclusters can be assembled on the surface of the conductive substrate. The conductive substrate can be organic or inorganic in nature, as long as it is able to conduct electrons through the material. The conductive substrate can be a polymeric conductor, a metallic conductor, a semiconductor, a carbon-based material, a metal oxide, or a modified conductor. The conductive substrate can be any suitable form such as a film, a mesh, or a disk. The conductive substrate can have any suitable shape such as regular shapes including, but not limited to, square, circle, triangle, and rectangle, and irregular shapes such as a waveform. In some instances, the conductive substrate is a printed electrode made of metals. In some instances, the printed electrode is disposable.

In some instances, the conductive substrate is made of a metallic conductor. Suitable metallic conductors include, but are not limited to, gold, chromium, platinum, iron, nickel, copper, silver, stainless steel, mercury, tungsten and other metals suitable for electrode construction. The metallic conductor can be a metal alloy which is made of a combination of metals disclosed herein. In addition, conductive substrates which are metallic conductors can be constructed of nanomaterials made of gold, cobalt, diamond, and other suitable metals. In some instances, the conductive substrate can be platinum. In some instances, the conductive substrate can be gold. In some instances, the conductive substrate can be silver.

In some instances, the conductive substrate is made from carbon-based materials. Exemplary carbon-based materials are carbon cloth, carbon paper, carbon screen printed electrodes, carbon paper, carbon black, carbon powder, carbon fiber, singe-walled carbon nanotubes, double-walled carbon nanotubes, multi-walled carbon nanotubes, carbon nanotube arrays, diamond-coated conductors, glassy carbon and mesoporous carbon. In addition, other exemplary carbon-based materials are graphene, graphite, uncompressed graphite worms, delaminated purified flake graphite, high performance graphite and carbon powders, highly ordered pyrolytic graphite, pyrolytic graphite, and polycrystalline graphite. In some instances, the conductive substrate can be printed carbon. In some instances, the conductive substrate can be glassy carbon.

In some instances, the conductive substrate can be a semiconductor. Suitable semiconductors are prepared from silicon and germanium, which can be doped (i.e., the intentional introduction of impurities into an intrinsic semiconductor for the purpose of modulating its electrical and structural properties) with other elements. The semiconductors can be doped with phosphorus, boron, gallium, arsenic, indium, antimony, or combinations thereof.

Other conductive substrate can be metal oxides, metal sulfides, main group compounds, and modified materials. Exemplary conductive substrates of this type include, but are not limited to, indium-tin-oxide (ITO) glass, nanoporous titanium oxide, tin oxide coated glass, cerium oxide particles, molybdenum sulfide, boron nitride nanotubes, aerogels modified with a conductive material such as gold, solgels modified with conductive material such as carbon, ruthenium carbon aerogels, and mesoporous silicas modified with a conductive material such as gold. In some instances, the conductive substrate is ITO glass.

In some instances, the conductive substrate contains one or more conducting materials. In instances where the conductive substrate contains two or more conducting materials, the first conducting material can be a conducting polymer and the second conducting material can be a different type of conducting material. The conducting polymers include but are not limited to poly(fluorine)s, polyphenylenes, polypyrenes, polyazulenes, polynaphthalenes, poly(pyrrole)s, polycarbozoles, polyindoles, polyzaepines, polyanilines, poly(thiophene)s, poly(3,4-ethylenedioxythiophene), poly(p-phenylene sulfide), poly(acetylene)s, poly(p-phenylene vinylene), and polyimides. The second conducting material can be sputter coated on top of the conducting polymer, and the aggregate of the two makes up the conductive substrate.

C. Coreactants

In some instances, the ECL sensors can include coreactants. The coreactants can be associated with the metal nanoclusters covalently or non-covalently, or not associated with the metal nanoclusters but can reach a close proximity to the metal nanoclusters to allow electron transfer between the metal nanoclusters and the coreactants. Exemplary coreactants include, but are not limited to, amines, oxalates, persulfates, hydrogen peroxide, nitrile, unsubstituted cyano, substituted cyano, unsubstituted benzophenone, substituted benzophenone, unsubstituted benzoic acid, substituted benzoic acid, unsubstituted naphthalene, substituted naphthalene, unsubstituted biphenyl, and substituted biphenyl. In some instances, the coreactants are amines. In some instances, the coreactants can be primary amines, secondary amines, tertiary amines, or combinations thereof. In some instances, the coreactants can be tertiary amines Exemplary tertiary amines include, but are not limited to, cetirizine, cetirizine derivative, tripropylamine, N,N-diethylethylenediamine, piperazine, and piperazine derivatives.

In some instances, the ECL sensors do not include coreactants. In some instances, the coreactants are covalently bound to the metal nanoclusters. The coreactants can be covalently bound to the core and/or the ligands of the metal nanoclusters. In some instances, the coreactants can be covalently bound to the core of the metal nanoclusters. In some instances, the coreactants can be covalently bound to the ligands of the metal nanoclusters. In some instances, the coreactants can be covalently bound to the core and the ligands of the metal nanoclusters. In some instances, the coreactants can be covalently attached to the termini of the ligands. Coupling reactions that can be used to covalently attach coreactants to the ligands are known in the art. For example, the coreactants can contain amine groups that react with the terminus carboxylic groups of the ligands to covalently attach the coreactants to the ligands of the metal nanoclusters. In some instances, the coreactants are covalently bound to the termini of the ligands of the metal nanoclusters and the metal nanoclusters are not Au₂₂LA₁₂.

In some instances, the coreactants can be associated with the core and/or ligands of the metal nanoclusters though non-covalent interactions. In some instances, the coreactants can be associate with the core of the metal nanoclusters through non-covalent interactions. In some instances, the coreactants can be associate with the ligands of the metal nanoclusters through non-covalent interactions. In some instances, the coreactants can be associate with the core and the ligands of the metal nanoclusters through non-covalent interactions. Exemplary non-covalent interactions include, but are not limited to, electrostatic, π effects, van der Waals forces, hydrogen bonding, and combinations thereof.

In some instances, the coreactants are not associated with the nanoclusters but can reach a close proximity to the metal nanoclusters to allow electron transfer between the coreactants and the metal nanoclusters.

In some instances, the coreactants can be assembled on the surface of the conductive substrate together with the metal nanoclusters. In some instances, the coreactants can be assembled on the surface of the conductive substrate without the metal nanoclusters on the surface of the conductive substrate.

In some instances, the coreactants are not assembled on the surface of the conductive substrate but can reach a close proximity to the surface of the conductive substrate to allow electron transfers between the coreactants and the conductive substrate. In some instances, the coreactants are not assembled on the surface of the conductive substrate and the metal nanoclusters are assembled on the surface of the conductive substrate. In some instances, both the coreactants and the metal nanoclusters are not assembled on the surface of the conductive substrate but can reach a close proximity to the surface of the conductive substrate to allow electron transfers between the coreactants and the conductive substrate and/or between the metal nanoclusters and the conductive substrate.

D. Reader

The ECL sensors and ECL sensing arrays can be connected to an acquisition system, optionally including a display component to form an ECL sensing system.

1. Acquisition System

An acquisition system can be a potentiostat, a power supply, or any suitable system to provide a potential to the ECL sensors or the ECL sensing array. The acquisition system can also include a detection component such as a camera or any suitable component to detect ECL generated from the ECL sensors or the ECL sensing array. Typically, the acquisition system can be connected to a software that can convert data into a graph, chart or table, for an analyte or a plurality of analytes.

2. Display Component

The display component can be a portable display system with a screen to display a sensor reading. Exemplary display systems include smartphones, tablets, laptops, desktops, and smartwatches, which are commercially available. The display systems typically include electronic conversion means, such as a software, to convert the signals received from the acquisition system to a concentration value or a graph, which can then be displayed on the screen. Such conversion means are known in the art.

E. Packaging

The ECL sensors and ECL sensing arrays can be packaged to protect the metal nanoclusters, the conductive substrates, and/or the coreactants prior to use. Examples of packaging are known in the art and include molded or sealed pouches with temperature and/or humidity control. The pouches can be foil pouches, paper pouches, cardboard boxes, polymeric pouches, or combinations thereof.

The ECL sensors, ECL sensing arrays, and reader can be packaged as one unit. Alternatively, the ECL sensors and ECL sensing arrays can be packaged separately from the reader, and used as needed with a reader containing an acquisition system and/or display components provided by the end users.

### III. Methods of Making the Electrochemiluminescence Sensors

ECL sensors described herein contain metal nanoclusters. In some instances, the ECL sensors can contain a conductive substrate. In some instances, the metal nanoclusters can be assembled on the surface of the conductive substrate. In some instances, the ECL sensors can further contain coreactants. In some instances, the coreactants can be covalently attached to the metal nanoclusters, associated with the metal nanoclusters non-covalently, or not associated with the metal nanoclusters. In some instances, the coreactants can be assembled on the surface of the conductive surface.

Metal nanoclusters can be prepared using any suitable methods known in the art or variations thereof. An exemplary general method is the size-focusing method (Zeng, et al., J. Am. Chem. Soc., 138(12):3950-3953 (2016); Zeng, et al., Sci. Adv., 1(2):e1500045 (2015)). The size-focusing method typically contains two steps: step (i) synthesize a controlled mixture of nanoclusters with a suitable size range, e.g., a size range with narrow distribution that covers the target size, which can be achieved by kinetic control, and step (ii) focus the mixed sizes into a single size by applying harsh reaction conditions, e.g., a high temperature between about 80 C and about 120° C. and excess thiols for chemical etching. The basic principle of size-focusing is that the harsh process selects the most stable size within the initially controlled size range in step (i). The synthesis of nanoclusters in step (i) typically include mixing metal salts with ligands followed by reducing the metal salts with a reductant such as NaBH₄ to form the core of the metal nanoclusters. Ligands are bound to the core through self-assembling reactions. In another exemplary method, AuAg nanoclusters can be prepared by a reaction between two metal nanocluster precursors, e.g., [Au₁₁(PPh₃)₈Cl₂]⁺ cluster and Ag(I)—SPhC₂H₄ complexes. An exemplary synthesis of the metal nanocluster precursors is described in Example 1 below. The metal nanoclusters can be characterized by Mass Spectrometry, UV-vis spectrometry, nuclear magnetic resonance, X-ray spectroscopy, Light scattering method using laser light, X-rays, and neutron scattering, IR spectroscopy, elemental analysis, and electrochemistry.

In some instances, coreactants can be covalently attached to the metal nanoclusters. For example, coreactants can be covalently or semi-covalently attached to the core of the metal nanoclusters through thiol groups or phosphino groups. In some instances, coreactants can be covalently attached to the ligands of the metal nanoclusters. For example, coreactants can be covalently attached to the terminus of the ligands of the metal nanoclusters through coupling reactions. Coupling reactions are known in the art. For example, the coreactant can contain amine groups that react with the terminus carboxylic groups of the ligands to covalently attach the coreactant to the ligands of the metal nanoclusters (Wang, et al., J. Am. Chem. Soc., 138(20):6380-6383 (2016)).

In some instances, the metal nanoclusters, coreactants, and/or conductive substrate are physically connected or in close proximity by placement into a sample. In some instances, the metal nanoclusters and/or coreactants can be assembled on the surface of the conductive substrate physically or chemically by any appropriate means. In some instances, the metal nanoclusters and/or coreactants can be dissolved in an appropriate solvent prior to the surface assembling. The solvent can be organic, aqueous, or a combination of both. Exemplary organic solvents include, but are not limited to, alcohols, esters, ethers, ketones, and nitrated and halogenated hydrocarbons, such as acetonitrile and methylene chloride, or a combination thereof. In some instances, solvent or a combination of solvents that have a slow evaporation rate is used for surface assembling of metal nanoclusters on the surface of the conductive substrate. For example, solvent or a mixture of two or more solvents that has an evaporation rate equal to or slower than the evaporation rate of DCM at room temperature can be used, such as ACN (boiling point 82° C.), chloroform (boiling point 60° C.), a mixture of DCM and ACN, or a mixture of DCM and chloroform. When a mixture of solvents is used to assemble the metal nanoclusters on the surface of the conductive substrate, the first solvent and the second solvent can have a volume ratio between 0.01 and 100, between 0.01 and 90, between 0.01 and 80, between 0.01 and 70, between 0.01 and 60, between 0.01 and 50, between 0.01 and 40, between 0.01 and 30, between 0.01 and 20, between 0.01 and 10, between 0.1 and 100, between 0.1 and 90, between 0.1 and 80, between 0.1 and 70, between 0.1 and 60, between 0.1 and 50, between 0.1 and 40, between 0.1 and 30, between 0.1 and 20, between 0.1 and 10, or between 0.1 and 5, such as 1. For example, a mixture of DCM:chloroform of 1:1 volume ratio can be used for assembling the metal nanoclusters on the surface of the conductive substrate. In some instances, the metal nanoclusters and/or coreactants are assembled on the surface of the conductive substrate physically by coating such as by spin-coating, dip-coating, drop-casting, Langmuir-Blogdett (L-B) type pulling, or electropolymerization, or otherwise deposing the individual components on the conductive substrate. In some instances, the solubilized metal nanoclusters and/or coreactants are deposited on the surface of the conductive substrate by a coating method described above, followed by drying at an appropriate temperature, e.g., at room temperature. In some instances, the metal nanoclusters and coreactants can be deposited separately, e.g., in layers, or they can be integrated into one deposition layer. In some instances, only metal nanoclusters are assembled on the surface of the conductive electrode by a coating method described herein.

Multiple drop addition, relative slow spin speed, and longer incubation time can be used to extend the sample-surface interaction time. Other solvents such as ACN (boiling point 82° C.) and chloroform (boiling point 60° C.) can be introduced. In addition to lower evaporation rates (i.e. slower evaporation), poorer solvent for the bimetallic NCs should also increase the NCs' affinity/interaction with ITO surface and self-assembly processes relative to DCM. Besides the slower evaporation rate of the solvent during spin coating, the changes in solvent polarity/affinity will also affect other interactions such as NCs with ITO surface and NCs themselves that affect the surface morphology or assembly pattern, and the correspondingly ECL and other properties. Spin-coating with mixed solvents to reduce the evaporation rate, the nanoclusters can self-assemble into microcrystals on the surface. Such nanocluster microcrystals, ordered assemblies on the surface, and their solid-state photoluminescence have not been previously observed. This discovery opens a new paradigm for the production and use of atomically precise nanoclusters, both from the fundamental perspective and for applications based on their physiochemical properties. The dimension of individual microcrystals, the distribution and coverage of the microcrystals, as well as their assembly can be optimized. Those depend on parameters such as the solvent ratio, nanocluster concentration, spin speed, drop volume, drop rate, and electrode surface preparations. Factors for choosing these parameters are illustrated in Example 6. Generally, slower solvent evaporation rate allows better interactions between the nanoclusters and the electron, which produces better surface distribution and coverage. For example, with slower evaporating mixed solvents, a faster spin speed and smaller drop volume produces a highly uniform film across a large coverage area on the electrode.

In some instances, the metal nanoclusters and/or coreactants can form a film on the surface of the conductive substrate. In some instances, the metal nanoclusters can form a film on the surface of the conductive substrate. In some instances, the metal nanoclusters can form a uniform film on the surface of the conductive substrate. In some instances, the metal nanoclusters can form a non-uniform film on the surface of the conductive substrate. In some instances, the distribution of the metal nanoclusters on a solid face depends on the relative hydrophilicity-hydrophobicity of the solvent used to dissolve the metal nanoclusters and/or coreactants, terminal groups of the metal nanoclusters, and the surface of the conductive substrate. In some instances, metal nanocluster films can be prepared by spin coating the solubilized metal nanoclusters in a solvent on the conductive substrate. In some instances, the speed of rotation during spin coating, metal nanoclusters concentration in the solvent, and the solvent evaporation rate can affect the uniformity of the metal nanocluster film on the surface of the conductive substrate. In some instances, the thickness of metal nanocluster films can be tuned by adjusting the metal nanoclusters concentration in the solvent and/or using layer-by-layer assembly.

In some instances, a protective layer such as oligoethylene glycol and polyethylene glycol (PEG) can be coated on top of the metal nanocluster film and/or the coreactant film. In some instances, a layer of PEG can be coated on top of the metal nanocluster and coreactant film. In some instances, a layer of polyethylene glycol can be coated on top of the metal nanocluster film. The protective film can reduce nonspecific adsorption of interferences in a sample, e.g., serum proteins in a biological sample.

In some instances, the film of metal nanoclusters can be characterized by electrochemical methods such as electrochemical impedance spectroscopy (EIS), cyclic voltammetry, linear sweeping voltammetry, differential pulse voltammetry, chronoamperometry, and amperometry. In some instances, the metal nanocluster film can be densely packed on the surface. In some instances, the metal nanocluster film can be porous. In some instances, the porous film is favorable because it allows the analytes in a sample to assess the metal nanoclusters and better interacts with the metal nanoclusters and the conductive substrate.

An acquisition system, such as a potentiostat or power supply as well as camera, is commercially available. In some instances, the acquisition system can contain a lead to connect the conductive substrate of the ECL sensor or the ECL sensing array to provide a potential. In some instances, the acquisition system can contain a conductive substrate to provide a potential to the ECL sensor. The acquisition system can also include a detection component such as a camera or any suitable component to detect ECL generated from the ECL sensors or the ECL sensing arrays. The acquisition system can then be connected to a display system, such as a device with a display screen. Exemplary display systems include smartphones, tablets, laptops, desktops, and smartwatches, are commercially available. The display systems typically include electronic conversion means, such as software, to convert the signals received from the acquisition system to a concentration value or a graph, which is then displayed on the screen. Such conversion means are known in the art.

### IV. Methods of Using the Electrochemiluminescence Sensors

The disclosed ECL sensors and ECL sensing arrays can be portable, wearable, or attachable to a subject. In some instances, the ECL sensors and ECL sensing arrays are small enough to be applied onto a medical device or onto a subject. The ECL sensors and ECL sensing arrays can be connected to an acquisition system such as a potentiostat or power supply, coupled with a camera, and, optionally, to a display system. The display system can be a portable display system with a screen to display sensor reading. Exemplary portable display systems include, but are not limited to, smartphones, tablets, laptops, desktop, pagers, watches, and glasses.

One of the various aspects of the disclosed ECL sensors is a method of testing the presence, absence, or concentration of an analyte of interest in a sample. In some instances, the ECL sensors permit effective sensing method using ECL that is rapid, simple, and allows for sensitive and specific detection of analytes of interest in a sample at a low cost. In some instances, methods of testing the presence, absence, or concentration of an analyte of interest in a sample can include: (i) contacting the sample with the ECL sensor, (ii) applying a potential to the sensor, and (iii) detecting the ECL and/or a redox current of the metal nanoclusters. In some instances, methods of screening the presence, absence, or concentration of a plurality of analytes of interest in a sample include: (i) contacting the sample with the ECL sensors of an ECL sensing array, (ii) applying a potential to the sensor, and (iii) detecting the ECL and/or redox currents of the metal nanoclusters. In some instances, the potential applied to each ECL sensors of the ECL sensing array can be the same. In some instances, the potential applied to each ECL sensors of the ECL sensing array can be different from each other.

In some instances, the potential applied to some of the ECL sensors of the ECL sensing array can be the same and the potential applied to other of the ECL sensors of the ECL sensing array can be different from one another. In some instances, the potential applied to some of the ECL sensors of the ECL sensing array can be the same and the potential applied to other of the ECL sensors of the ECL sensing array can be different from each other and from the ECL sensor of the array having the same applied potential. In some instances, the potential applied to some of the ECL sensors of the ECL sensing array can be the same and the potential applied to other of the ECL sensors of the ECL sensing array can be different from every other ECL sensor in the array. In some instances, the potential applied to one or more sets of the ECL sensors of the ECL sensing array can be the same as the ECL sensors in the same set. In some instances, the different of such sets of ECL sensors in the array can have a different applied potential than the ECL sensors in the other sets.

In some instances, the ECL sensors contain metal nanoclusters assembled on the surface of a conductive substrate. In some instances, the surface assembled metal nanoclusters can be coated with a protective layer such as a layer of PEG. In some instances, the metal nanoclusters of the ECL sensor can get in close proximity to a conductive substrate when placed into a sample to allow electron transfer between the conductive substrate and the metal nanoclusters. In some instances, the metal nanoclusters and coreactants of the ECL sensor can get in close proximity to a conductive substrate when placed into a sample to allow electron transfer among the metal nanoclusters, the coreactants, and the conductive substrate.

In some instances, the potential can be applied by linearly sweeping from a first potential to a second potential. In some instances, the potential can be applied by linear sweeping from a first potential to a second potential, then to a third potential, where the third potential is different from the first and second potential. In some instances, the potential can be applied by linear sweeping from a first potential to a second potential, from the second potential to a third potential, then from the third potential to a fourth potential, where the third potential is different from the first and second potential, and where the fourth potential is different from the third potential. In some instances, the fourth potential can be the same as or different from the first and/or second potential. In some instances, the fourth potential is different from the first, second, and third potential. In some instances, the fourth potential can be the same as the first potential and different from the third potential. In some instances, the fourth potential can be the same as the second potential and different from the third potential.

Generally, in the linear sweeping method, the second potential is not the same as the first potential, the third potential is not the same as the second potential, and the fourth potential is not the same as the third potential.

In some instances, the potential can be applied by linearly sweeping between a first potential and a second potential in cycles (cyclic sweeping). In some instances, the potential can be applied by cyclic sweeping between a first potential and a second potential, then linear sweeping from the second potential to a third potential, where the third potential is different from the first potential. In some instances, one cycle of sweeping is when the potential is swept from the first potential to the second potential and returns to the first potential. In some instances, the number of cycles can be at least 1, at least 2, at least 3, at least 5, at least 10, at least 15, at least 20, at least 30, at least 40, at least 50, at least 60, at least 70, at least 80, at least 90, or up to 100, such as 1, 2, 3, 4, 5, 6, 7, 8, 9, 10, 11, 15, 20, 25, 30, 35, 40, 45, 50, 55, 60, 65, 70, 75, 80, 85, 90, 95, or 100. In some instances, the number of cycles is 2. In some instances, the number of cycles is 4. In some instances, the number of cycles is 5.

In some instances, the potential can be applied by linear sweeping from a first potential to a second potential, then cyclic sweeping between the second potential and a third potential, where the third potential is different from the first potential. In such instances, one cycle of sweeping is when the potential is swept from the second potential to the third potential and returns to the second potential. In some instances, the number of cycles can be at least 1, at least 2, at least 3, at least 5, at least 10, at least 15, at least 20, at least 30, at least 40, at least 50, at least 60, at least 70, at least 80, at least 90, or up to 100, such as 1, 2, 3, 4, 5, 6, 7, 8, 9, 10, 11, 15, 20, 25, 30, 35, 40, 45, 50, 55, 60, 65, 70, 75, 80, 85, 90, 95, or 100. In some instances, the number of cycles is 2. In some instances, the number of cycles is 4. In some instances, the number of cycles is 5.

In some instances, the potential can be applied by linear sweeping from a first potential to a second potential, then from the second potential to a third potential, followed by cyclic sweeping between the third potential and a fourth potential, where the third potential is different from the first potential and where the fourth potential is different from the third potential. In such instances, one cycle of sweeping is when the potential is swept from the third potential to the fourth potential and returns to the third potential. In some instances, the number of cycles can be at least 1, at least 2, at least 3, at least 5, at least 10, at least 15, at least 20, at least 30, at least 40, at least 50, at least 60, at least 70, at least 80, at least 90, or up to 100, such as 1, 2, 3, 4, 5, 6, 7, 8, 9, 10, 11, 15, 20, 25, 30, 35, 40, 45, 50, 55, 60, 65, 70, 75, 80, 85, 90, 95, or 100. In some instances, the number of cycles is 2. In some instances, the number of cycles is 4. In some instances, the number of cycles is 5.

Generally, in the cyclic sweeping method, the second potential is not the same as the first potential, the third potential is not the same as the second potential, and the fourth potential is not the same as the third potential.

In some instances, the potential can be applied by stepping between a first potential and a second potential cyclically, where the first potential and the second potential in a cycle is each held for a time period. In some instances, one cycle of stepping is when the potential is stepped from the first potential to the second potential and returns to the first potential. In some instances, the potential can be applied by stepping from a first potential to a second potential, then from the second potential to a third potential, where the third potential is different from the first potential. In such instances, one cycle of stepping is when the potential is stepped from the first potential to the second potential, from the second potential to the third potential, then returns from the third potential to the first potential. The cycle can be repeated and each potential in a cycle is held for a time period.

In some instances, the potential can be applied by stepping from a first potential to a second potential, from the second potential to a third potential, then from the third potential to a fourth potential, where the third potential can be the same as or different from the first potential, and where the fourth potential can be the same as or different from the first and second potential. In some instances, the third potential can be the same as the first potential and the fourth potential can be different from the first and second potentials. In some instances, the third potential can be different from the first potential and the fourth potential can different from the first, second, and third potential. In some instances, the third potential can be different from the first potential and the fourth potential can be the same as the second potential. Generally, in the stepping method, the second potential is not the same as the first potential, the third potential is not the same as the second potential, and the fourth potential is not the same as the third potential. In such instances, one cycle of stepping is when the potential is stepped from the first potential to the second potential, from the second potential to the third potential, from the third potential to the fourth potential, then returns from the fourth potential to the first potential. The cycle can be repeated and each potential in a cycle is held for a time period.

In some instances, the number of cycles for potential stepping can be at least 1, at least 2, at least 3, at least 5, at least 10, at least 15, at least 20, at least 30, at least 40, at least 50, at least 60, at least 70, at least 80, at least 90, at least 100, at least 150, at least 200, at least 240, at least 300, at least 400, or up to 500, such as 1, 2, 3, 4, 5, 6, 7, 8, 9, 10, 11, 15, 20, 25, 30, 35, 40, 45, 50, 55, 60, 65, 70, 75, 80, 85, 90, 95, 100, 150, 200, or 240. In some instances, the number of cycles is 1. In some instances, the number of cycles is 2. In some instances, the number of cycles is 3. In some instances, the number of cycles is 4. In some instances, the number of cycles is 5. In some instances, the number of cycles is 10. In some instances, the number of cycles is 20. In some instances, the number of cycles is 50. In some instances, the number of cycles is 100. In some instances, the number of cycles is 200. In some instances, the number of cycles is 240. In some instances, the ECL signal of the metal nanoclusters can be stable for at least 10 cycles, at least at least 15, at least 20, at least 30, at least 40, at least 50, at least 60, at least 70, at least 80, at least 90, at least 100, at least 150, at least 200, at least 240, at least 300, at least 400, or up to 500 cycles of potential stepping. “Stable” generally means that the change of ECL intensity of the metal nanoclusters during measurement is equal to or less than 10% of the ECL intensity measured from the second cycle. In some instances, the ECL signal generated from metal nanoclusters assembled on a conductive substrate surface, such as a metal nanocluster film on the conductive substrate surface, can be stable for at least 10 cycles, at least at least 15, at least 20, at least 30, at least 40, at least 50, at least 60, at least 70, at least 80, at least 90, at least 100, at least 150, at least 200, at least 240, at least 300, at least 400, or up to 500 cycles of potential stepping. For example, the ECL signal generated from a metal nanocluster film assembled on a conductive substrate surface can be stable for at least 240 cycles of potential stepping.

In some instances, the time period for holding each potential in a cycle can be the same. In some instances, the time period for holding each potential can be different. In some instances, the time period for holding the first potential is different from the time period for holding the second and third potential, where the time period for holding the second and the third potential is the same. In some instances, the time period for holding the second potential is different from the time period for holding the first and third potential, where the time period for holding the first and the third potential is the same. In some instances, the time period for holding the third potential is different from the time period for holding the first and second potential, where the time period for holding the first and the second potential is the same. In some instances, the time period for holding the first potential, second potential, and third potential is different from one another. In some instances, the time period for holding the first potential is different from the time period for holding the second, third, and fourth potential, where the time period for holding the second, third, and fourth potential is the same. In some instances, the time period for holding the second potential is different from the time period for holding the first, third, and fourth potential, where the time period for holding the first, third, and fourth potential is the same. In some instances, the time period for holding the second and third potential is the same and the time period for holding the first and fourth potential is the same but different from the time period for holding the first and the third potential. In some instances, the time period for holding the first and second potential is the same and the time period for holding the third and fourth potential is the same but different from the time period for holding the first and the second potential. In some instances, the time period for holding the first and second potential is the same and different from the time period for holding the third and the fourth potential, where the time periods for holding the third and the fourth potential are different.

In some instances, the time period for holding each potential can be independently between about 0.01 s and about 5000 s, between about 0.01 s and about 2400 s, between about 0.01 s and about 1200 s, between about 0.01 s and about 1000 s, between about 0.01 s and about 600 s, between about 0.01 s and about 500 s, between about 0.01 s and about 400 s, between about 0.01 s and about 240 s, between about 0.01 s and about 120 s, between about 0.01 s and about 100 s, between about 0.01 s and about 60 s, between about 0.01 s and about 30 s, between about 0.01 s and about 20 s, between about 0.01 s and about 10 s, between about 0.01 s and about 5 s, between about 0.05 s and about 240 s, between about 0.05 s and about 120 s, between about 0.05 s and about 60 s, between about 0.05 s and about 30 s, between about 0.05 s and about 20 s, between about 0.05 s and about 10 s, between about 0.05 s and about 5 s, between about 0.1 s and about 240 s, between about 0.1 s and about 120 s, between about 0.1 s and about 60 s, between about 0.1 s and about 30 s, between about 0.1 s and about 20 s, between about 0.1 s and about 10 s, between about 0.1 s and about 5 s, between about 0.2 s and about 240 s, between about 0.2 s and about 120 s, between about 0.2 s and about 60 s, between about 0.2 s and about 30 s, between about 0.2 s and about 20 s, between about 0.2 s and about 10 s, between about 0.2 s and about 5 s, between about 0.5 s and about 240 s, between about 0.5 s and about 120 s, between about 0.5 s and about 60 s, between about 0.5 s and about 30 s, between about 0.5 s and about 20 s, between about 0.5 s and about 10 s, between about 0.5 s and about 5 s, between about 1 s and about 240 s, between about 1 s and about 120 s, between about 1 s and about 60 s, between about 1 s and about 30 s, between about 1 s and about 20 s, between about 1 s and about 10 s, or between about 1 s and about 5 s. In some instances, the time period for holding each potential can be between about 0.1 s and about 10 s. In some instances, the time period for holding each potential can be between about 0.1 s and about 8 s. In some instances, the time period for holding each potential can be between about 0.1 s and about 5 s. the time period for holding each potential can be between about 0.1 s and about 3 s. the time period for holding each potential can be between about 0.1 s and about 2 s. the time period for holding each potential can be between about 0.1 s and about 1 s. In some instances, the time period for holding each potential can be between about 1 s and about 10 s. In some instances, the time period for holding each potential can be between about 1 s and about 8 s. In some instances, the time period for holding each potential can be between about 1 s and about 6 s. In some instances, the time period for holding each potential can be between about 1 s and about 5 s. In some instances, the time period for holding each potential can be between about 1 s and about 3 s.

In some instances, the first potential and the second potential in the stepping method are each held for about 5 seconds in a cycle. In some instances, the first potential and the second potential in the stepping method are each held for about 0.2 seconds in a cycle. In some instances, the first potential in the stepping method is held for 0.3 second and the second potential is held for 0.1 second in a cycle. In some instances, the first, second, and third potential in the stepping method are each held for about 5 seconds in a cycle. In some instances, the first, second, third, and fourth potential in the stepping method are each held for about 5 seconds in a cycle. In some instances, the first and second potential in the stepping method are each held for about 5 seconds and the third and fourth potential are each held for about 3 seconds in a cycle. In some instances, the first and second potential in the stepping method are each held for about 5 seconds, the third potential is held for 1 seconds, and the fourth potential is held for about 3 seconds in a cycle.

In some instances, the first potential and the second potential in the stepping method are each held for 5 seconds in a cycle. In some instances, the first, second, and third potential in the stepping method are each held for 5 seconds in a cycle. In some instances, the first, second, third, and fourth potential in the stepping method are each held for 5 seconds in a cycle. In some instances, the first and second potential in the stepping method are each held for 5 seconds and the third and fourth potential are each held for 3 seconds in a cycle. In some instances, the first and second potential in the stepping method are each held for 5 seconds, the third potential is held for 1 seconds, and the fourth potential is held for 3 seconds in a cycle.

The metal nanoclusters can generate self-annihilation ECL, coreactant ECL, or a combination thereof. In some instances, the metal nanoclusters are capable of generating ECL in the absence of coreactants (i.e. self-annihilation ECL). In some instances, the self-annihilation ECL intensity from the metal nanoclusters is at least 2 times, at least 3 times, at least 5 times, at least 10 times, at least 12 times, at least 15 times, at least 20 times, at least 25 times, at least 30 times, at least 40 times, at least 50 times, at least 100 times higher than that from Ru(bpy)₃ under the same conditions. In an exemplary case, the self-annihilation ECL intensity from AgₓAu₂₅₋ₓ nanoclusters (x is a positive integer ≤13) (e.g., Au₁₂Ag₁₃ nanoclusters) is about ten times higher than that from Ru(bpy)₃ under the same conditions. In some instances, the metal nanoclusters are capable of generating ECL in the presence of a coreactant (i.e. coreactant ECL). In some instances, the coreactant ECL intensity from the metal nanoclusters is at least 10 times, at least 20 times, at least 50 times, at least 100 times, at least 120 times, at least 150 times, at least 200 times, at least 250 times, at least 300 times, at least 350 times, or at least 400 times higher than that from Ru(bpy)₃ under the same conditions. In an exemplary case, with a coreactant such as tripropylamine (TPrA), the coreactant ECL of AgₓAu₂₅₋ₓ nanoclusters (x is a positive integer ≤13) (e.g., Au₁₂Ag₁₃ nanoclusters) is about 400 times higher than Ru(bpy)₃ under the same conditions.

In some instances, the strong ECL of the metal nanoclusters can be attributed to one or more metal atoms in the metal core of the metal nanoclusters that produce stability of the metal core. For example, it has been discovered that the strong ECL of Au₁₂Ag₁₃ nanoclusters can be attributed to the 13th Ag atom at the central position. Without being bound to a particular theory of operation, this central Ag atom appears to stabilize the charges on LUMO orbital and makes the rod-shape Ag₁₃Au₁₂ core more rigid. Thus, arrangements of metal atoms that produce similar stability can also produce higher ECL. Such metal nanoclusters with high ECL provide new tools in applications such as sensing and assay analysis.

In some instances, the potential applied to the ECL sensor is sufficient to provide enough energy to activate the corresponding energy states of the metal nanoclusters, the coreactant, the analytes, or combinations thereof. In some instances, the applied potential is sufficient to provide enough energy to activate the corresponding energy states of the metal nanoclusters. For example, the first potential and the second potential cover the first oxidation peak and the first reduction peak of the metal nanoclusters respectively, and vice versa. In some instances, the applied potential is sufficient to provide enough energy to activate the corresponding energy states of the coreactant. In some instances, the applied potential is sufficient to provide enough energy to activate the corresponding energy states of the analytes. In some instances, the applied potential is sufficient to provide enough energy to activate the corresponding energy states of the metal nanoclusters and the coreactants. In some instances, the applied potential is sufficient to provide enough energy to activate the corresponding energy states of the metal nanoclusters and the analytes. In some instances, the applied potential is sufficient to provide enough energy to activate the corresponding energy states of the metal nanoclusters, the coreactants, and the analytes.

In some instances, the potential applied to the ECL sensor that is sufficient to provide enough energy to activate the corresponding energy states of the metal nanoclusters, the coreactant, the analytes, or combinations thereof is less negative than −1.8 V, less negative than −1.7 V, less negative than −1.6 V, or less negative than-1.5 V versus a reference electrode, such as a Ag/AgCl reference electrode. In some instances, the potential applied to the ECL sensor that is sufficient to provide enough energy to activate the corresponding energy states of the metal nanoclusters, the coreactant, the analytes, or combinations thereof is between about −1.5 V and about 2 V, between about −1.5 V and about 1.9 V, between about −1.5 V and about 1.8 V, between about −1.5 V and about 1.7 V, between about −1.5 V and about 1.6 V, between about −1.5 V and about 1.5 V, between about −1.5 V and about 1.4 V, between about −1.5 V and about 1.3 V, between about −1.3 V and about 1.3 V, between about −1.2 V and about 1.2 V, between about −1.1 V and about 1.1 V, between about −1.0 V and about 1.0 V, between about −1.2 V and about 1.1 V, between about −1.2 V and about 1.0 V, between about −1.2 V and about 0.9 V, between about −1.2 V and about 0.8 V, between about −1.1V and about 1.0 V, between about −1.1 V and about 0.9 V, between about −1.1 V and about 0.8 V, between about −1.0 V and about 0.9 V, between about −1.0 V and about 0.8 V, between about −1.1 V and about 1.2 V, between about −1.0 V and about 1.2 V, between about −0.9 V and about 1.2 V, between about −0.8 V and about 1.2 V, between about −1.0 V and about 1.1 V, between about −0.9 V and about 1.1 V, between about −0.8 V and about 1.1 V, between about −0.9 V and about 1.0 V, between about −0.8 V and about 1.0 V, between about −0.9 V and about 0.9 V, between about −0.9 V and about 0.8 V, between about −0.8 V and about 0.9 V, between about −0.8 V and about 0.8 V, versus a reference electrode, such as a Ag/AgCl reference electrode.

In some instances, the potential applied to the ECL sensor is sufficient to provide enough energy to activate one or more of the corresponding energy states of the metal nanoclusters, the coreactant, the analytes, or combinations thereof. In some instances, the applied potential is sufficient to provide enough energy to activate one or more of the corresponding energy states of the metal nanoclusters. For example, the first potential and the second potential cover one or more oxidation peaks and one or more reduction peaks of the metal nanoclusters respectively, and vice versa. In some instances, the applied potential is sufficient to provide enough energy to activate one or more of the corresponding energy states of the coreactant. In some instances, the applied potential is sufficient to provide enough energy to activate one or more of the corresponding energy states of the analytes. In some instances, the applied potential is sufficient to provide enough energy to activate one or more of the corresponding energy states of the metal nanoclusters and the coreactants. In some instances, the applied potential is sufficient to provide enough energy to activate one or more of the corresponding energy states of the metal nanoclusters and the analytes. In some instances, the applied potential is sufficient to provide enough energy to activate one or more of the corresponding energy states of the metal nanoclusters, the coreactants, and the analytes.

In some instances, the potential applied to the ECL sensor that is sufficient to provide enough energy to activate one or more of the corresponding energy states of the metal nanoclusters, the coreactant, the analytes, or combinations thereof is less negative than-1.8 V, less negative than-1.7 V, less negative than-1.6 V, or less negative than-1.5 V versus a reference electrode, such as a Ag/AgCl reference electrode. In some instances, the potential applied to the ECL sensor that is sufficient to provide enough energy to activate one or more of the corresponding energy states of the metal nanoclusters, the coreactant, the analytes, or combinations thereof is between about −1.5 V and about 2 V, between about −1.5 V and about 1.9 V, between about −1.5 V and about 1.8 V, between about −1.5 V and about 1.7 V, between about −1.5 V and about 1.6 V, between about −1.5 V and about 1.5 V, between about −1.5 V and about 1.4 V, between about −1.5 V and about 1.3 V, between about −1.3 V and about 1.3 V, between about −1.2 V and about 1.2 V, between about −1.1 V and about 1.1 V, between about −1.0 V and about 1.0 V, between about −1.2 V and about 1.1 V, between about −1.2 V and about 1.0 V, between about −1.2 V and about 0.9 V, between about −1.2 V and about 0.8 V, between about −1.1V and about 1.0 V, between about −1.1 V and about 0.9 V, between about −1.1 V and about 0.8 V, between about −1.0 V and about 0.9 V, between about −1.0 V and about 0.8 V, between about −1.1 V and about 1.2 V, between about −1.0 V and about 1.2 V, between about −0.9 V and about 1.2 V, between about −0.8 V and about 1.2 V, between about −1.0 V and about 1.1 V, between about −0.9 V and about 1.1 V, between about −0.8 V and about 1.1 V, between about −0.9 V and about 1.0 V, between about −0.8 V and about 1.0 V, between about −0.9 V and about 0.9 V, between about −0.9 V and about 0.8 V, between about −0.8 V and about 0.9 V, between about −0.8 V and about 0.8 V, versus a reference electrode, such as a Ag/AgCl reference electrode.

In some instances, the potential applied to the ECL sensor is sufficient to provide enough energy to activate a particular corresponding energy state of the metal nanoclusters, the coreactant, the analytes, or combinations thereof. In some instances, the applied potential is sufficient to provide enough energy to activate a particular corresponding energy state of the metal nanoclusters. For example, the first potential and the second potential cover a particular oxidation peak and a particular reduction peak of the metal nanoclusters respectively, and vice versa. In some instances, the applied potential is sufficient to provide enough energy to activate a particular corresponding energy state of the coreactant. In some instances, the applied potential is sufficient to provide enough energy to activate a particular corresponding energy state of the analytes. In some instances, the applied potential is sufficient to provide enough energy to activate a particular corresponding energy state of the metal nanoclusters and the coreactants. In some instances, the applied potential is sufficient to provide enough energy to activate a particular corresponding energy state of the metal nanoclusters and the analytes. In some instances, the applied potential is sufficient to provide enough energy to activate a particular corresponding energy state of the metal nanoclusters, the coreactants, and the analytes.

In some instances, the potential applied to the ECL sensor that is sufficient to provide enough energy to activate a particular corresponding energy states of the metal nanoclusters, the coreactant, the analytes, or combinations thereof is less negative than-1.8 V, less negative than-1.7 V, less negative than-1.6 V, or less negative than-1.5 V versus a reference electrode, such as a Ag/AgCl reference electrode. In some instances, the potential applied to the ECL sensor that is sufficient to provide enough energy to activate a particular corresponding energy state of the metal nanoclusters, the coreactant, the analytes, or combinations thereof is between about −1.5 V and about 2 V, between about −1.5 V and about 1.9 V, between about −1.5 V and about 1.8 V, between about −1.5 V and about 1.7 V, between about −1.5 V and about 1.6 V, between about −1.5 V and about 1.5 V, between about −1.5 V and about 1.4 V, between about −1.5 V and about 1.3 V, between about −1.3 V and about 1.3 V, between about −1.2 V and about 1.2 V, between about −1.1 V and about 1.1 V, between about −1.0 V and about 1.0 V, between about −1.2 V and about 1.1 V, between about −1.2 V and about 1.0 V, between about −1.2 V and about 0.9 V, between about −1.2 V and about 0.8 V, between about −1.1V and about 1.0 V, between about −1.1 V and about 0.9 V, between about −1.1 V and about 0.8 V, between about −1.0 V and about 0.9 V, between about −1.0 V and about 0.8 V, between about −1.1 V and about 1.2 V, between about −1.0 V and about 1.2 V, between about −0.9 V and about 1.2 V, between about −0.8 V and about 1.2 V, between about −1.0 V and about 1.1 V, between about −0.9 V and about 1.1 V, between about −0.8 V and about 1.1 V, between about −0.9 V and about 1.0 V, between about −0.8 V and about 1.0 V, between about −0.9 V and about 0.9 V, between about −0.9 V and about 0.8 V, between about −0.8 V and about 0.9 V, between about −0.8 V and about 0.8 V, versus a reference electrode, such as a Ag/AgCl reference electrode.

In some instances, the potential applied to the ECL sensor is sufficient to provide enough energy to activate one of the corresponding energy states of the metal nanoclusters, the coreactant, the analytes, or combinations thereof. In some instances, the applied potential is sufficient to provide enough energy to activate one of the corresponding energy states of the metal nanoclusters. For example, the first potential and the second potential cover one of the oxidation peaks and one of the reduction peaks of the metal nanoclusters respectively, and vice versa. In some instances, the applied potential is sufficient to provide enough energy to activate one of the corresponding energy states of the coreactant. In some instances, the applied potential is sufficient to provide enough energy to activate one of the corresponding energy states of the analytes. In some instances, the applied potential is sufficient to provide enough energy to activate one of the corresponding energy states of the metal nanoclusters and the coreactants. In some instances, the applied potential is sufficient to provide enough energy to activate one of the corresponding energy states of the metal nanoclusters and the analytes. In some instances, the applied potential is sufficient to provide enough energy to activate one of the corresponding energy states of the metal nanoclusters, the coreactants, and the analytes.

In some instances, the potential applied to the ECL sensor that is sufficient to provide enough energy to activate one of the corresponding energy states of the metal nanoclusters, the coreactant, the analytes, or combinations thereof is less negative than-1.8 V, less negative than-1.7 V, less negative than-1.6 V, or less negative than-1.5 V versus a reference electrode, such as a Ag/AgCl reference electrode. In some instances, the potential applied to the ECL sensor that is sufficient to provide enough energy to activate one of the corresponding energy states of the metal nanoclusters, the coreactant, the analytes, or combinations thereof is between about −1.5 V and about 2 V, between about −1.5 V and about 1.9 V, between about −1.5 V and about 1.8 V, between about −1.5 V and about 1.7 V, between about −1.5 V and about 1.6 V, between about −1.5 V and about 1.5 V, between about −1.5 V and about 1.4 V, between about −1.5 V and about 1.3 V, between about −1.3 V and about 1.3 V, between about −1.2 V and about 1.2 V, between about −1.1 V and about 1.1 V, between about −1.0 V and about 1.0 V, between about −1.2 V and about 1.1 V, between about −1.2 V and about 1.0 V, between about −1.2 V and about 0.9 V, between about −1.2 V and about 0.8 V, between about −1.1V and about 1.0 V, between about −1.1 V and about 0.9 V, between about −1.1 V and about 0.8 V, between about −1.0 V and about 0.9 V, between about −1.0 V and about 0.8 V, between about −1.1 V and about 1.2 V, between about −1.0 V and about 1.2 V, between about −0.9 V and about 1.2 V, between about −0.8 V and about 1.2 V, between about −1.0 V and about 1.1 V, between about −0.9 V and about 1.1 V, between about −0.8 V and about 1.1 V, between about −0.9 V and about 1.0 V, between about −0.8 V and about 1.0 V, between about −0.9 V and about 0.9 V, between about −0.9 V and about 0.8 V, between about −0.8 V and about 0.9 V, between about −0.8 V and about 0.8 V, versus a reference electrode, such as a Ag/AgCl reference electrode.

In some instances, the potential applied to the ECL sensor is sufficient to provide enough energy to activate a plurality of the corresponding energy states of the metal nanoclusters, the coreactant, the analytes, or combinations thereof. In some instances, the applied potential is sufficient to provide enough energy to activate a plurality of the corresponding energy states of the metal nanoclusters. For example, the first potential and the second potential cover a plurality of oxidation peaks and a plurality of reduction peaks of the metal nanoclusters respectively, and vice versa. In some instances, the applied potential is sufficient to provide enough energy to activate a plurality of the corresponding energy states of the coreactant. In some instances, the applied potential is sufficient to provide enough energy to activate a plurality of the corresponding energy states of the analytes. In some instances, the applied potential is sufficient to provide enough energy to activate a plurality of the corresponding energy states of the metal nanoclusters and the coreactants. In some instances, the applied potential is sufficient to provide enough energy to activate a plurality of the corresponding energy states of the metal nanoclusters and the analytes. In some instances, the applied potential is sufficient to provide enough energy to activate a plurality of the corresponding energy states of the metal nanoclusters, the coreactants, and the analytes.

In some instances, the potential applied to the ECL sensor that is sufficient to provide enough energy to activate a plurality of the corresponding energy states of the metal nanoclusters, the coreactant, the analytes, or combinations thereof is less negative than-1.8 V, less negative than-1.7 V, less negative than-1.6 V, or less negative than-1.5 V versus a reference electrode, such as a Ag/AgCl reference electrode. In some instances, the potential applied to the ECL sensor that is sufficient to provide enough energy to activate a plurality of the corresponding energy states of the metal nanoclusters, the coreactant, the analytes, or combinations thereof is between about −1.5 V and about 2 V, between about −1.5 V and about 1.9 V, between about −1.5 V and about 1.8 V, between about −1.5 V and about 1.7 V, between about −1.5 V and about 1.6 V, between about −1.5 V and about 1.5 V, between about −1.5 V and about 1.4 V, between about −1.5 V and about 1.3 V, between about −1.3 V and about 1.3 V, between about −1.2 V and about 1.2 V, between about −1.1 V and about 1.1 V, between about −1.0 V and about 1.0 V, between about −1.2 V and about 1.1 V, between about −1.2 V and about 1.0 V, between about −1.2 V and about 0.9 V, between about −1.2 V and about 0.8 V, between about −1.1V and about 1.0 V, between about −1.1 V and about 0.9 V, between about −1.1 V and about 0.8 V, between about −1.0 V and about 0.9 V, between about −1.0 V and about 0.8 V, between about −1.1 V and about 1.2 V, between about −1.0 V and about 1.2 V, between about −0.9 V and about 1.2 V, between about −0.8 V and about 1.2 V, between about −1.0 V and about 1.1 V, between about −0.9 V and about 1.1 V, between about −0.8 V and about 1.1 V, between about −0.9 V and about 1.0 V, between about −0.8 V and about 1.0 V, between about −0.9 V and about 0.9 V, between about −0.9 V and about 0.8 V, between about −0.8 V and about 0.9 V, between about −0.8 V and about 0.8 V, versus a reference electrode, such as a Ag/AgCl reference electrode.

In some instances, the potential applied to the ECL sensor that is sufficient to provide enough energy to activate the corresponding energy states of the metal nanoclusters is less negative than-1.8 V, less negative than-1.7 V, less negative than-1.6 V, or less negative than-1.5 V versus a reference electrode, such as a Ag/AgCl reference electrode. In some instances, the potential applied to the ECL sensor that is sufficient to provide enough energy to activate the corresponding energy states of the metal nanoclusters is between about −1.5 V and about 2 V, between about −1.5 V and about 1.9 V, between about −1.5 V and about 1.8 V, between about −1.5 V and about 1.7 V, between about −1.5 V and about 1.6 V, between about −1.5 V and about 1.5 V, between about −1.5 V and about 1.4 V, between about −1.5 V and about 1.3 V, between about −1.3 V and about 1.3 V, between about −1.2 V and about 1.2 V, between about −1.1 V and about 1.1 V, between about −1.0 V and about 1.0 V, between about −1.2 V and about 1.1 V, between about −1.2 V and about 1.0 V, between about −1.2 V and about 0.9 V, between about −1.2 V and about 0.8 V, between about −1.1V and about 1.0 V, between about −1.1 V and about 0.9 V, between about −1.1 V and about 0.8 V, between about −1.0 V and about 0.9 V, between about −1.0 V and about 0.8 V, between about −1.1 V and about 1.2 V, between about −1.0 V and about 1.2 V, between about −0.9 V and about 1.2 V, between about −0.8 V and about 1.2 V, between about −1.0 V and about 1.1 V, between about −0.9 V and about 1.1 V, between about −0.8 V and about 1.1 V, between about −0.9 V and about 1.0 V, between about −0.8 V and about 1.0 V, between about −0.9 V and about 0.9 V, between about −0.9 V and about 0.8 V, between about −0.8 V and about 0.9 V, between about −0.8 V and about 0.8 V, versus a Ag/AgCl reference electrode. In some instances, the potential applied to the ECL sensor that is enough to cover at least one of the oxidation peaks of the metal nanoclusters respectively is less than 2 V, less than 1.9 V, less than 1.8 V, less than 1.7 V, less than 1.6 V, less than 1.5 V, less than 1.4 V, less than 1.3 V, less than 1.2 V, less than 1.1 V, less than 1.0 V, less than 0.9 V, or less than 0.8 V versus a Ag/AgCl reference electrode, for example, between 0 V and 2 V, between 0 V and 1.9 V, between 0 V and 1.8 V, between 0 V and 1.7 V, between 0 V and 1.6 V, between 0 V and 1.5 V, between 0 V and 1.4 V, between 0 V and 1.3 V, between 0 V and 1.2 V, between 0 V and 1.1 V, or between 0 V and 1 V versus a Ag/AgCl reference electrode. In some instances, the potential applied to the ECL sensor that is enough to cover at least one of the reduction peaks of the metal nanoclusters respectively is less negative than-1.8 V, less negative than-1.7 V, less negative than-1.6 V, less negative than-1.5 V, less negative than-1.4 V, less negative than-1.3 V, less negative than-1.2 V, less negative than-1.1 V, less negative than-1.0 V, less negative than-0.9 V, or less negative than-0.8 V versus a Ag/AgCl reference electrode, for example, between −1.7 V and 0 V, between-1.6 V and 0 V, between-1.5 V and 0 V, between-1.4 V and 0 V, or between-1.3 V and 0 V versus a Ag/AgCl reference electrode. In some instances, the potential applied to the ECL sensor that is enough to cover the dominant oxidation peak of the metal nanoclusters respectively is less than 2 V, less than 1.9 V, less than 1.8 V, less than 1.7 V, less than 1.6 V, less than 1.5 V, less than 1.4 V, less than 1.3 V, less than 1.2 V, less than 1.1 V, less than 1.0 V, less than 0.9 V, or less than 0.8 V versus a Ag/AgCl reference electrode, for example, between 0 V and 2 V, between 0 V and 1.9 V, between 0 V and 1.8 V, between 0 V and 1.7 V, between 0 V and 1.6 V, between 0 V and 1.5 V, between 0 V and 1.4 V, between 0 V and 1.3 V, between 0 V and 1.2 V, between 0 V and 1.1 V, or between 0 V and 1 V versus a Ag/AgCl reference electrode. In some instances, the potential applied to the ECL sensor that is enough to cover the dominant reduction peak of the metal nanoclusters respectively is less negative than-1.8 V, less negative than-1.7 V, less negative than-1.6 V, less negative than-1.5 V, less negative than-1.4 V, less negative than-1.3 V, less negative than-1.2 V, less negative than-1.1 V, less negative than-1.0 V, less negative than-0.9 V, or less negative than-0.8 V versus a Ag/AgCl reference electrode, for example, between-1.7 V and 0 V, between-1.6 V and 0 V, between-1.5 V and 0 V, between-1.4 V and 0 V, or between-1.3 V and 0 V versus a Ag/AgCl reference electrode. The term “dominant oxidation peak” generally refers to the peak having the highest oxidation current among all oxidation peaks. The term “dominant reduction peak” generally refers to the peak having the highest reduction current among all reduction peaks.

In some instances, the potential applied to the ECL sensor is to generate self-annihilation ECL. Generally, ECL signals generated from self-annihilation pathway need both reduced metal nanoclusters and oxidized metal nanoclusters produced by a first potential and a second potential. Typically, the first potential is a negative potential that is sufficient to provide enough energy to activate the corresponding energy states of the metal nanocluster (e.g., to reduce the metal nanoclusters) or to activate the most dominant reduction peak of the metal nanoclusters and the second potential is a positive potential that is sufficient to provide enough energy to activate the corresponding energy states of the metal nanocluster (e.g., to oxidize the metal nanoclusters) or to activate the most dominant oxidation peak of the metal nanoclusters. Alternatively, the first potential is a positive potential that is sufficient to provide enough energy to activate the corresponding energy states of the metal nanocluster (e.g., to oxidize the metal nanoclusters) or to activate the most dominant oxidation peak of the metal nanoclusters and the second potential is a negative potential that is sufficient to provide enough energy to activate the corresponding energy states of the metal nanocluster (e.g., to reduce the metal nanoclusters) or to activate the most dominant reduction peak of the metal nanoclusters. In some instances, the negative potential sufficient to provide enough energy to activate the corresponding energy states of the metal nanoclusters (e.g., reduce the metal nanoclusters) is less negative than-1.8 V, less negative than-1.7 V, less negative than-1.6 V, less negative than-1.5 V, less negative than-1.4 V, less negative than-1.3 V, less negative than-1.2 V, less negative than-1.1 V, less negative than-1.0 V, less negative than-0.9 V, less negative than-0.8 V, less negative than-0.7 V, less negative than-0.6 V, or less negative than-0.5 V versus a reference electrode, such as a Ag/AgCl reference electrode. In some instances, the negative potential sufficient to provide enough energy to activate the most dominant reduction peak of the metal nanoclusters (e.g., reduce the metal nanoclusters) is less negative than-1.8 V, less negative than-1.7 V, less negative than-1.6 V, less negative than-1.5 V, less negative than-1.4 V, less negative than-1.3 V, less negative than-1.2 V, less negative than-1.1 V, less negative than-1.0 V, less negative than-0.9 V, less negative than-0.8 V, less negative than-0.7 V, less negative than-0.6 V, or less negative than-0.5 V versus a reference electrode, such as a Ag/AgCl reference electrode. In some instances, the positive potential sufficient to provide enough energy to activate the corresponding energy states of the metal nanoclusters (e.g., oxidize the metal nanoclusters) is less than 2 V, less than 1.9 V, less than 1.8 V, less than 1.7 V, less than 1.6 V, less than 1.5 V, less than 1.4 V, less than 1.3 V, less than 1.2 V, less than 1.1 V, less than 1.0 V, less than 0.9 V, less than 0.8 V, less than 0.7 V, less than 0.6 V, or less than 0.5 V versus a reference electrode, such as a Ag/AgCl reference electrode. In some instances, the positive potential sufficient to provide enough energy to activate the corresponding energy states of the metal nanoclusters (e.g., oxidize the metal nanoclusters) is less than 2 V, less than 1.9 V, less than 1.8 V, less than 1.7 V, less than 1.6 V, less than 1.5 V, less than 1.4 V, less than 1.3 V, less than 1.2 V, less than 1.1 V, less than 1.0 V, less than 0.9 V, less than 0.8 V, less than 0.7 V, less than 0.6 V, or less than 0.5 V versus a reference electrode, such as a Ag/AgCl reference electrode. In some instances, the positive potential sufficient to provide enough energy to activate the most dominant oxidation peak of the metal nanoclusters (e.g., oxidize the metal nanoclusters) is less than 2 V, less than 1.9 V, less than 1.8 V, less than 1.7 V, less than 1.6 V, less than 1.5 V, less than 1.4 V, less than 1.3 V, less than 1.2 V, less than 1.1 V, less than 1.0 V, less than 0.9 V, less than 0.8 V, less than 0.7 V, less than 0.6 V, or less than 0.5 V versus a reference electrode, such as a Ag/AgCl reference electrode. Each of the first potential and the second potential can be independently held for any time periods described above. In some instances, the first potential is held for a time period longer than the second potential. In some instances, the first potential is held for a time period the same or substantially the same as the second potential.

In some instances, the analytes can interact with the metal nanoclusters and/or the coreactants of the ECL sensors. In some instances, the analytes can interact with the metal nanoclusters. In some instances, the analytes can interact with the coreactants of the ECL sensors. In some instances, the analytes can interact with the metal nanoclusters and the coreactants of the ECL sensors. In some instances, the interactions between the analytes and/or the metal nanoclusters and/or the coreactants of the ECL sensors include non-covalent interactions as described herein. In some instances, the interactions between the analytes and/or the metal nanoclusters and/or the coreactants of the ECL sensors are electron transfers between the analytes and/or the metal nanoclusters. In some instances, the ECL sensors does not include coreactants. In some instances, the ECL sensors include metal nanoclusters assembled on the surface of a conductive substrate and does not include coreactants. In some instances, the ECL sensors include organo-soluble metal nanoclusters assembled on the surface of a conductive substrate and does not include coreactants. In some instances, the ECL sensors include metal nanoclusters and coreactants. In some instances, the ECL sensors include aqueous soluble metal nanoclusters and coreactants. In some instances, the ECL sensors include aqueous soluble metal nanoclusters and coreactants covalently attached to the metal nanoclusters. The level of increase or decrease of the ECL of the metal nanoclusters is correlated to the concentration of the analyte.

In some instances, the ECL of the metal nanoclusters increases or decreases upon an interaction between the analyte and the metal nanoclusters and/or the coreactants as compared to the ECL of the metal nanoclusters in the absence of the analyte. In some instances, the ECL of the metal nanoclusters increases or decreases upon an interaction between the analyte and the metal nanoclusters as compared to the ECL of the metal nanoclusters in the absence of the analyte. In some instances, the ECL of the organo-soluble metal nanoclusters increases or decreases upon an interaction between the analyte and the organo-soluble metal nanoclusters as compared to the ECL of the metal nanoclusters in the absence of the analyte. In some instances, the ECL of the metal nanoclusters increases or decreases upon an interaction between the analyte, the metal nanoclusters and the coreactants as compared to the ECL of the metal nanoclusters in the absence of the analyte. In some instances, the ECL of the aqueous soluble metal nanoclusters increases or decreases upon an interaction between the analyte, the metal nanoclusters, and the coreactants as compared to the ECL of the metal nanoclusters in the absence of the analyte. The level of increase or decrease of the ECL of the metal nanoclusters is correlated to the concentration of the analyte.

In some instances, the ECL of the metal nanoclusters can increase or decrease over a period of time upon an interaction between the analyte and the metal nanoclusters and/or the coreactant as compared to the ECL of the metal nanoclusters in the absence of the analyte. In some instances, the ECL of the metal nanoclusters can increase or decrease over a period of time upon an interaction between the analyte and the metal nanoclusters as compared to the ECL of the metal nanoclusters in the absence of the analyte. In some instances, the ECL of the metal nanoclusters can increase or decrease over a period of time upon an interaction between the analyte, the metal nanoclusters, and the coreactant as compared to the ECL of the metal nanoclusters in the absence of the analyte. In some instances, the ECL of the organo-soluble metal nanoclusters can increase or decrease over a period of time upon an interaction between the analyte and the organo-soluble metal nanoclusters and/or the coreactant as compared to the ECL of the organo-soluble metal nanoclusters in the absence of the analyte. In some instances, the ECL of the organo-soluble metal nanoclusters can increase or decrease over a period of time upon an interaction between the analyte and the organo-soluble metal nanoclusters as compared to the ECL of the organo-soluble metal nanoclusters in the absence of the analyte. In some instances, the ECL of the organo-soluble metal nanoclusters can increase or decrease over a period of time upon an interaction between the analyte, the organo-soluble metal nanoclusters, and the coreactant as compared to the ECL of the organo-soluble metal nanoclusters in the absence of the analyte. In some instances, the level of increase or decrease of the ECL of the metal nanoclusters over a period of time can be correlated to the concentration and/or amount of the analyte. In some instances, the increases or decreases of the ECL of the organo-soluble metal nanoclusters over a period of time can be correlated to the concentration and/or amount of the analyte. For example, the ECL intensity of the metal nanoclusters can be measured every 1 second, every 0.5 second, every 0.2 second, every 0.1 second, every 0.05 second, every 0.02 second, or every 0.01 second upon the addition of an analyte, which can be plotted as a function of time (intensity-time curve), where the slope of the intensity-time curve can be correlated to the concentration of the analyte.

In some instances, the time period over which the ECL of the metal nanoclusters can increase or decrease upon an interaction between the metal nanoclusters and the analyte and/or the coreactant can be between about 1 second and about 30 minutes, between about 1 second and about 25 minutes, between about 1 second and about 20 minutes, between about 1 minute and about 30 minutes, between about 1 minute and about 25 minutes, between about 1 minute and about 20 minutes, between about 1 minute and about 15 minutes, between about 1 minute and about 10 minutes, between about 1 minute and about 5 minutes, between about 2 minute and about 30 minutes, between about 2 minute and about 25 minutes, between about 2 minute and about 20 minutes, between about 2 minute and about 15 minutes, between about 2 minute and about 10 minutes, between about 5 minute and about 30 minutes, between about 5 minute and about 25 minutes, between about 5 minute and about 20 minutes, between about 5 minute and about 15 minutes, 5 minute and about 10 minutes, between about 1 second and about 10 minutes, between about 1 second and about 5 minutes, between about 1 second and about 4 minutes, between about 1 second and about 3 minutes, between about 1 second and about 2 minutes, between about 1 second and about 1.5 minutes, between about 1 second and about 1 minutes, between about 1 second and about 50 seconds, between about 1 second and about 45 seconds, between about 1 second and about 40 seconds, between about 1 second and about 35 seconds, between about 1 second and about 30 seconds, between about 1 second and about 25 seconds, between about 1 second and about 20 seconds, between about 1 second and about 15 seconds, between about 1 second and about 10 seconds, between about 1 second and about 8 seconds, between about 1 second and about 6 seconds, between about 1 second and about 5 seconds, between about 1 second and about 4 seconds, between about 1 second and about 3 seconds, between about 1 second and about 2 seconds, between about 0.1 second and about 2 seconds, between about 0.1 second and about 1 second, between about 0.1 second and about 0.5 second, between about 0.01 second and about 1 second, between about 0.01 second and about 0.5 second, between about 0.01 second and about 0.2 second, between about 0.01 second and about 0.1 second, between about 0.01 second and about 0.05 second.

In some instances, the time period over which the ECL of the organo-soluble metal nanoclusters can increase or decrease upon an interaction between the organo-soluble metal nanoclusters and the analyte, and/or the coreactant can be between about 1 second and about 30 minutes, between about 1 second and about 25 minutes, between about 1 second and about 20 minutes, between about 1 minute and about 30 minutes, between about 1 minute and about 25 minutes, between about 1 minute and about 20 minutes, between about 1 minute and about 15 minutes, between about 1 minute and about 10 minutes, between about 1 minute and about 5 minutes, between about 2 minute and about 30 minutes, between about 2 minute and about 25 minutes, between about 2 minute and about 20 minutes, between about 2 minute and about 15 minutes, between about 2 minute and about 10 minutes, between about 5 minute and about 30 minutes, between about 5 minute and about 25 minutes, between about 5 minute and about 20 minutes, between about 5 minute and about 15 minutes, 5 minute and about 10 minutes, between about 1 second and about 10 minutes, between about 1 second and about 5 minutes, between about 1 second and about 4 minutes, between about 1 second and about 3 minutes, between about 1 second and about 2 minutes, between about 1 second and about 1.5 minutes, between about 1 second and about 1 minutes, between about 1 second and about 50 seconds, between about 1 second and about 45 seconds, between about 1 second and about 40 seconds, between about 1 second and about 35 seconds, between about 1 second and about 30 seconds, between about 1 second and about 25 seconds, between about 1 second and about 20 seconds, between about 1 second and about 15 seconds, between about 1 second and about 10 seconds, between about 1 second and about 8 seconds, between about 1 second and about 6 seconds, between about 1 second and about 5 seconds, between about 1 second and about 4 seconds, between about 1 second and about 3 seconds, between about 1 second and about 2 seconds, between about 0.1 second and about 2 seconds, between about 0.1 second and about 1 second, between about 0.1 second and about 0.5 second, between about 0.01 second and about 1 second, between about 0.01 second and about 0.5 second, between about 0.01 second and about 0.2 second, between about 0.01 second and about 0.1 second, between about 0.01 second and about 0.05 second.

In some instances, the redox current of the metal nanoclusters increases or decreases upon an interaction between the analyte and the metal nanoclusters and/or the coreactants as compared to the redox current of the metal nanoclusters in the absence of the analyte. In some instances, the redox current of the metal nanoclusters increases or decreases upon an interaction between the analyte and the metal nanoclusters as compared to the redox current of the metal nanoclusters in the absence of the analyte. In some instances, the redox current of the organo-soluble metal nanoclusters increases or decreases upon an interaction between the analyte and the organo-soluble metal nanoclusters as compared to the redox current of the metal nanoclusters in the absence of the analyte. In some instances, the redox current of the metal nanoclusters increases or decreases upon an interaction between the analyte, the metal nanoclusters and the coreactants as compared to the redox current of the metal nanoclusters in the absence of the analyte. In some instances, the redox current of the aqueous soluble metal nanoclusters increases or decreases upon an interaction between the analyte, the metal nanoclusters, and the coreactants as compared to the redox current of the metal nanoclusters in the absence of the analyte. The level of increase or decrease of the redox current of the metal nanoclusters is correlated to the concentration of the analyte.

In some instances, the ECL of the metal nanoclusters increases upon an interaction between the analyte and the metal nanoclusters and/or the coreactants as compared to the ECL of the metal nanoclusters in the absence of the analyte. In some instances, the ECL of the metal nanoclusters increases upon an interaction between the analyte and the metal nanoclusters as compared to the ECL of the metal nanoclusters in the absence of the analyte. In some instances, the ECL of the organo-soluble metal nanoclusters increases upon an interaction between the analyte and the organo-soluble metal nanoclusters as compared to the ECL of the metal nanoclusters in the absence of the analyte. In some instances, the ECL of the metal nanoclusters increases upon an interaction between the analyte, the metal nanoclusters and the coreactants as compared to the ECL of the metal nanoclusters in the absence of the analyte. In some instances, the ECL of the aqueous soluble metal nanoclusters increases upon an interaction between the analyte, the metal nanoclusters, and the coreactants as compared to the ECL of the metal nanoclusters in the absence of the analyte. The level of increase of the ECL of the metal nanoclusters is correlated to the concentration of the analyte.

In some instances, the ECL of the metal nanoclusters increases over a period of time upon an interaction between the analyte and the metal nanoclusters and/or the coreactant as compared to the ECL of the metal nanoclusters in the absence of the analyte. In some instances, the ECL of the metal nanoclusters increases over a period of time upon an interaction between the analyte and the metal nanoclusters as compared to the ECL of the metal nanoclusters in the absence of the analyte. In some instances, the ECL of the metal nanoclusters increases over a period of time upon an interaction between the analyte, the metal nanoclusters, and the coreactant as compared to the ECL of the metal nanoclusters in the absence of the analyte. In some instances, the ECL of the organo-soluble metal nanoclusters increases over a period of time upon an interaction between the analyte and the organo-soluble metal nanoclusters and/or the coreactant as compared to the ECL of the organo-soluble metal nanoclusters in the absence of the analyte. In some instances, the ECL of the organo-soluble metal nanoclusters increases over a period of time upon an interaction between the analyte and the organo-soluble metal nanoclusters as compared to the ECL of the organo-soluble metal nanoclusters in the absence of the analyte. In some instances, the ECL of the organo-soluble metal nanoclusters increases over a period of time upon an interaction between the analyte, the organo-soluble metal nanoclusters, and the coreactant as compared to the ECL of the organo-soluble metal nanoclusters in the absence of the analyte. In some instances, the increases of the ECL of the metal nanoclusters over a period of time can be correlated to the concentration and/or amount of the analyte. In some instances, the increases of the ECL of the organo-soluble metal nanoclusters over a period of time can be correlated to the concentration and/or amount of the analyte. For example, the ECL intensity of the metal nanoclusters can be measured every 1 second, every 0.5 second, every 0.2 second, every 0.1 second, every 0.05 second, every 0.02 second, or every 0.01 second upon the addition of an analyte, which can be plotted as a function of time (intensity-time curve), where the slope of the intensity-time curve can be correlated to the concentration of the analyte.

In some instances, the time period over which the ECL of the metal nanoclusters increases upon an interaction between the analyte and the metal nanoclusters, and/or coreactant can be between about 1 second and about 30 minutes, between about 1 second and about 25 minutes, between about 1 second and about 20 minutes, between about 1 minute and about 30 minutes, between about 1 minute and about 25 minutes, between about 1 minute and about 20 minutes, between about 1 minute and about 15 minutes, between about 1 minute and about 10 minutes, between about 1 minute and about 5 minutes, between about 2 minute and about 30 minutes, between about 2 minute and about 25 minutes, between about 2 minute and about 20 minutes, between about 2 minute and about 15 minutes, between about 2 minute and about 10 minutes, between about 5 minute and about 30 minutes, between about 5 minute and about 25 minutes, between about 5 minute and about 20 minutes, between about 5 minute and about 15 minutes, 5 minute and about 10 minutes, between about 1 second and about 10 minutes, between about 1 second and about 5 minutes, between about 1 second and about 4 minutes, between about 1 second and about 3 minutes, between about 1 second and about 2 minutes, between about 1 second and about 1.5 minutes, between about 1 second and about 1 minutes, between about 1 second and about 50 seconds, between about 1 second and about 45 seconds, between about 1 second and about 40 seconds, between about 1 second and about 35 seconds, between about 1 second and about 30 seconds, between about 1 second and about 25 seconds, between about 1 second and about 20 seconds, between about 1 second and about 15 seconds, between about 1 second and about 10 seconds, between about 1 second and about 8 seconds, between about 1 second and about 6 seconds, between about 1 second and about 5 seconds, between about 1 second and about 4 seconds, between about 1 second and about 3 seconds, between about 1 second and about 2 seconds, between about 0.1 second and about 2 seconds, between about 0.1 second and about 1 second, between about 0.1 second and about 0.5 second, between about 0.01 second and about 1 second, between about 0.01 second and about 0.5 second, between about 0.01 second and about 0.2 second, between about 0.01 second and about 0.1 second, between about 0.01 second and about 0.05 second.

In some instances, the time period over which the ECL of the organo-soluble metal nanoclusters increases upon an interaction between the organo-soluble metal nanoclusters and the analyte, and/or the coreactant can be between about 1 second and about 30 minutes, between about 1 second and about 25 minutes, between about 1 second and about 20 minutes, between about 1 minute and about 30 minutes, between about 1 minute and about 25 minutes, between about 1 minute and about 20 minutes, between about 1 minute and about 15 minutes, between about 1 minute and about 10 minutes, between about 1 minute and about 5 minutes, between about 2 minute and about 30 minutes, between about 2 minute and about 25 minutes, between about 2 minute and about 20 minutes, between about 2 minute and about 15 minutes, between about 2 minute and about 10 minutes, between about 5 minute and about 30 minutes, between about 5 minute and about 25 minutes, between about 5 minute and about 20 minutes, between about 5 minute and about 15 minutes, 5 minute and about 10 minutes, between about 1 second and about 10 minutes, between about 1 second and about 5 minutes, between about 1 second and about 4 minutes, between about 1 second and about 3 minutes, between about 1 second and about 2 minutes, between about 1 second and about 1.5 minutes, between about 1 second and about 1 minutes, between about 1 second and about 50 seconds, between about 1 second and about 45 seconds, between about 1 second and about 40 seconds, between about 1 second and about 35 seconds, between about 1 second and about 30 seconds, between about 1 second and about 25 seconds, between about 1 second and about 20 seconds, between about 1 second and about 15 seconds, between about 1 second and about 10 seconds, between about 1 second and about 8 seconds, between about 1 second and about 6 seconds, between about 1 second and about 5 seconds, between about 1 second and about 4 seconds, between about 1 second and about 3 seconds, between about 1 second and about 2 seconds, between about 0.1 second and about 2 seconds, between about 0.1 second and about 1 second, between about 0.1 second and about 0.5 second, between about 0.01 second and about 1 second, between about 0.01 second and about 0.5 second, between about 0.01 second and about 0.2 second, between about 0.01 second and about 0.1 second, between about 0.01 second and about 0.05 second.

In some instances, the redox current of the metal nanoclusters increases upon an interaction between the analyte and the metal nanoclusters and/or the coreactants as compared to the redox current of the metal nanoclusters in the absence of the analyte. In some instances, the redox current of the metal nanoclusters increases upon an interaction between the analyte and the metal nanoclusters as compared to the redox current of the metal nanoclusters in the absence of the analyte. In some instances, the redox current of the organo-soluble metal nanoclusters increases upon an interaction between the analyte and the organo-soluble metal nanoclusters as compared to the redox current of the metal nanoclusters in the absence of the analyte. In some instances, the redox current of the metal nanoclusters increases upon an interaction between the analyte, the metal nanoclusters and the coreactants as compared to the redox current of the metal nanoclusters in the absence of the analyte. In some instances, the redox current of the aqueous soluble metal nanoclusters increases upon an interaction between the analyte, the metal nanoclusters, and the coreactants as compared to the redox current of the metal nanoclusters in the absence of the analyte. The level of increase of the redox current of the metal nanoclusters is correlated to the concentration of the analyte.

In some instances, the ECL of the metal nanoclusters decreases upon an interaction between the analyte and the metal nanoclusters and/or the coreactants as compared to the ECL of the metal nanoclusters in the absence of the analyte. In some instances, the ECL of the metal nanoclusters decreases upon an interaction between the analyte and the metal nanoclusters as compared to the ECL of the metal nanoclusters in the absence of the analyte. In some instances, the ECL of the organo-soluble metal nanoclusters decreases upon an interaction between the analyte and the organo-soluble metal nanoclusters as compared to the ECL of the metal nanoclusters in the absence of the analyte. In some instances, the ECL of the metal nanoclusters decreases upon an interaction between the analyte, the metal nanoclusters and the coreactants as compared to the ECL of the metal nanoclusters in the absence of the analyte. In some instances, the ECL of the aqueous soluble metal nanoclusters decreases upon an interaction between the analyte, the metal nanoclusters, and the coreactants as compared to the ECL of the metal nanoclusters in the absence of the analyte. The level of decrease of the ECL of the metal nanoclusters is correlated to the concentration of the analyte.

In some instances, the ECL of the metal nanoclusters decreases over a period of time upon an interaction between the analyte and the metal nanoclusters and/or the coreactant as compared to the ECL of the metal nanoclusters in the absence of the analyte. In some instances, the ECL of the metal nanoclusters decreases over a period of time upon an interaction between the analyte and the metal nanoclusters as compared to the ECL of the metal nanoclusters in the absence of the analyte. In some instances, the ECL of the metal nanoclusters decreases over a period of time upon an interaction between the analyte, the metal nanoclusters, and the coreactant as compared to the ECL of the metal nanoclusters in the absence of the analyte. In some instances, the ECL of the organo-soluble metal nanoclusters decreases over a period of time upon an interaction between the analyte and the organo-soluble metal nanoclusters and/or the coreactant as compared to the ECL of the organo-soluble metal nanoclusters in the absence of the analyte. In some instances, the ECL of the organo-soluble metal nanoclusters decreases over a period of time upon an interaction between the analyte and the organo-soluble metal nanoclusters as compared to the ECL of the organo-soluble metal nanoclusters in the absence of the analyte. In some instances, the ECL of the organo-soluble metal nanoclusters decreases over a period of time upon an interaction between the analyte, the organo-soluble metal nanoclusters, and the coreactant as compared to the ECL of the organo-soluble metal nanoclusters in the absence of the analyte. In some instances, the decreases of the ECL of the metal nanoclusters over a period of time can be correlated to the concentration and/or amount of the analyte. In some instances, the decreases of the ECL of the organo-soluble metal nanoclusters over a period of time can be correlated to the concentration and/or amount of the analyte. For example, the ECL intensity of the metal nanoclusters can be measured every 1 second, every 0.5 second, every 0.2 second, every 0.1 second, every 0.05 second, every 0.02 second, or every 0.01 second upon the addition of an analyte, which can be plotted as a function of time (intensity-time curve), where the slope of the intensity-time curve can be correlated to the concentration and/or amount of the analyte.

In some instances, the time period over which the ECL of the metal nanoclusters decreases upon an interaction between the metal nanoclusters and the analyte and/or the coreactant can be between about 1 second and about 30 minutes, between about 1 second and about 25 minutes, between about 1 second and about 20 minutes, between about 1 minute and about 30 minutes, between about 1 minute and about 25 minutes, between about 1 minute and about 20 minutes, between about 1 minute and about 15 minutes, between about 1 minute and about 10 minutes, between about 1 minute and about 5 minutes, between about 2 minute and about 30 minutes, between about 2 minute and about 25 minutes, between about 2 minute and about 20 minutes, between about 2 minute and about 15 minutes, between about 2 minute and about 10 minutes, between about 5 minute and about 30 minutes, between about 5 minute and about 25 minutes, between about 5 minute and about 20 minutes, between about 5 minute and about 15 minutes, 5 minute and about 10 minutes, between about 1 second and about 10 minutes, between about 1 second and about 5 minutes, between about 1 second and about 4 minutes, between about 1 second and about 3 minutes, between about 1 second and about 2 minutes, between about 1 second and about 1.5 minutes, between about 1 second and about 1 minutes, between about 1 second and about 50 seconds, between about 1 second and about 45 seconds, between about 1 second and about 40 seconds, between about 1 second and about 35 seconds, between about 1 second and about 30 seconds, between about 1 second and about 25 seconds, between about 1 second and about 20 seconds, between about 1 second and about 15 seconds, between about 1 second and about 10 seconds, between about 1 second and about 8 seconds, between about 1 second and about 6 seconds, between about 1 second and about 5 seconds, between about 1 second and about 4 seconds, between about 1 second and about 3 seconds, between about 1 second and about 2 seconds, between about 0.1 second and about 2 seconds, between about 0.1 second and about 1 second, between about 0.1 second and about 0.5 second, between about 0.01 second and about 1 second, between about 0.01 second and about 0.5 second, between about 0.01 second and about 0.2 second, between about 0.01 second and about 0.1 second, between about 0.01 second and about 0.05 second.

In some instances, the time period over which the ECL of the organo-soluble metal nanoclusters decreases upon an interaction between the organo-soluble metal nanoclusters and the analyte and/or the coreactant can be between about 1 second and about 30 minutes, between about 1 second and about 25 minutes, between about 1 second and about 20 minutes, between about 1 minute and about 30 minutes, between about 1 minute and about 25 minutes, between about 1 minute and about 20 minutes, between about 1 minute and about 15 minutes, between about 1 minute and about 10 minutes, between about 1 minute and about 5 minutes, between about 2 minute and about 30 minutes, between about 2 minute and about 25 minutes, between about 2 minute and about 20 minutes, between about 2 minute and about 15 minutes, between about 2 minute and about 10 minutes, between about 5 minute and about 30 minutes, between about 5 minute and about 25 minutes, between about 5 minute and about 20 minutes, between about 5 minute and about 15 minutes, 5 minute and about 10 minutes, between about 1 second and about 10 minutes, between about 1 second and about 5 minutes, between about 1 second and about 4 minutes, between about 1 second and about 3 minutes, between about 1 second and about 2 minutes, between about 1 second and about 1.5 minutes, between about 1 second and about 1 minutes, between about 1 second and about 50 seconds, between about 1 second and about 45 seconds, between about 1 second and about 40 seconds, between about 1 second and about 35 seconds, between about 1 second and about 30 seconds, between about 1 second and about 25 seconds, between about 1 second and about 20 seconds, between about 1 second and about 15 seconds, between about 1 second and about 10 seconds, between about 1 second and about 8 seconds, between about 1 second and about 6 seconds, between about 1 second and about 5 seconds, between about 1 second and about 4 seconds, between about 1 second and about 3 seconds, between about 1 second and about 2 seconds, between about 0.1 second and about 2 seconds, between about 0.1 second and about 1 second, between about 0.1 second and about 0.5 second, between about 0.01 second and about 1 second, between about 0.01 second and about 0.5 second, between about 0.01 second and about 0.2 second, between about 0.01 second and about 0.1 second, between about 0.01 second and about 0.05 second.

In some instances, the redox current of the metal nanoclusters decreases upon an interaction between the analyte and the metal nanoclusters and/or the coreactants as compared to the redox current of the metal nanoclusters in the absence of the analyte. In some instances, the redox current of the metal nanoclusters decreases upon an interaction between the analyte and the metal nanoclusters as compared to the redox current of the metal nanoclusters in the absence of the analyte. In some instances, the redox current of the organo-soluble metal nanoclusters decreases upon an interaction between the analyte and the organo-soluble metal nanoclusters as compared to the redox current of the metal nanoclusters in the absence of the analyte. In some instances, the redox current of the metal nanoclusters decreases upon an interaction between the analyte, the metal nanoclusters and the coreactants as compared to the redox current of the metal nanoclusters in the absence of the analyte. In some instances, the redox current of the aqueous soluble metal nanoclusters decreases upon an interaction between the analyte, the metal nanoclusters, and the coreactants as compared to the redox current of the metal nanoclusters in the absence of the analyte. The level of decrease of the redox current of the metal nanoclusters is correlated to the concentration of the analyte.

Methods for determining the energy states of a species are known in the art, such as Electrochemical methods and absorption spectroscopy in UV-vis-near IR range (Wang, et al., JACS, 138:6380-6383 (2016); Wang, et al., Chem Electro Chem, 4:1697-1701 (2017); and Wang, et al., Electrochimica Acta, 282:369-376 (2018)). For example, the energy states of the metal nanoclusters, the coreactants, and/or the analytes can be determined by measuring the redox currents of the metal nanoclusters, the coreactants, and/or the analytes at different applied potentials. In some instances, the redox currents of the metal nanoclusters, the coreactants, and/or the analytes were measured by linearly sweeping from a first potential to a second potential. In some instances, the redox currents of the metal nanoclusters, the coreactants, and/or the analytes were measured by linearly sweeping between a first potential and a second potential in cycles. In some instances, the appearance of an oxidation and/or reduction peak is indicative of the corresponding energy states of the metal nanoclusters, the coreactants, and/or the analytes.

In some instances, the ECL and the redox current of the ECL sensors can be measured simultaneously. In some instances, the ECL and the redox current of the ECL sensors can be measured separately. In some instances, the ECL and the redox current of the ECL sensors can be measured sequentially. In some instances, the ECL of the ECL sensors can be measured before the redox current of the ECL sensors is measured. In some instances, the redox current of the ECL sensors can be measured before the ECL of the ECL sensors is measured. In some instances, the ECL and redox current of the ECL sensors measured can be used together to improve the specificity of the ECL sensors. For example, the ECL and redox current (I) of the ECL sensors measured can have a ratio ECL/I, which captures sufficient kinetics profiles of a particular analyte to improve the ECL sensor specificity.

Generally, ECL generation involves multiple steps and the mechanism is more complex as compared to photoluminescence. ECL generation can follow a self-annihilation pathway or a coreactant pathway. Generally, it is the case that a high photoluminescence (PL) intensity of a metal nanocluster does not correlate with a high ECL intensity of the metal nanocluster. It is known that the signal generation mechanisms for PL and ECL are fundamentally different. See, for example, Miao, Chemical Reviews, 108(7):2506-2553 (2008). Without being bound to a particular theory of operation, ECL generation involves more complex reactions compared to PL. For example, self-annihilation ECL generated from the oxidation and reduction of the same ECL reagents suffers from self-quenching of radical species. When coreactants are used, the addition of millimolar to submolar coreactants complicates the detection system and ECL generation mechanisms. For example, metal nanoclusters with strong PL may have negligible (i.e., near zero) ECL due to side reactions and/or insufficient electrode potentials.

An exemplary self-annihilation ECL pathway for metal nanoclusters (NC) is shown below:

| | |
|---|---|
| [NCs] + e⁻ → [NCs]^{-•} | (a) |
| [NCs]^{-•} − e⁻ → [NCs]^{*} | (b) |
| [NCs] − e⁻ → [NCs]^{+•} | (c) |
| [NCs]^{+•} + e⁻ → [NCs]^{*} | (d) |
| [NCs]^{-•} + [NCs]^{+•} → [NCs]^{*} + [NCs] | (e) |
| [NCs]^{*} → [NCs] + hv | (f) |

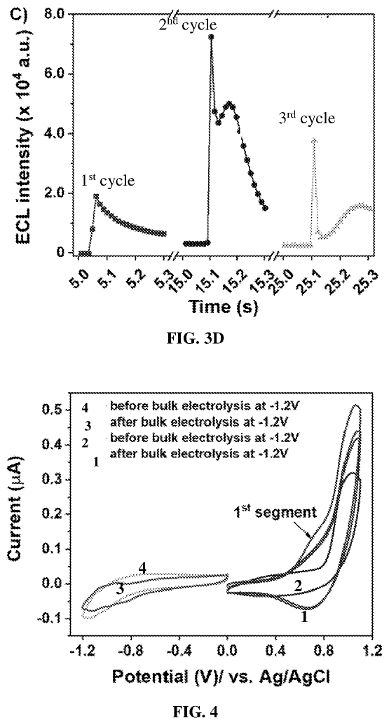

In steps a & c, the reduced and oxidized species are generated by electrode electron transfer reactions. Step e is the diffusive reaction between the two types of radical intermediates, after which the excited [NC]* releases the energy via photon emission (step f). The mechanism for the oxidative ECL peak is depicted in FIG. 6A. An electron is firstly injected into the LUMO of NCs such as Au₁₂Ag₁₃ (step a). Those cathodically produced radicals will be oxidized once the electrode potential is stepped below HOMO (step b). Loss of HOMO electron directly produces the excited species [NC]* which relaxes to the ground state and emits light. This pathway only involves those pre-reduced NCs directly at the electrode surface vicinity. Further, loss of the electron in LUMO after step a, however, will quench the emission process. Therefore, the ECL displays a transient peak which quickly decays within milliseconds. Similarly, FIG. 6B depicts the reductive ECL processes. Loss of HOMO electron (step c) followed by the reduction to LUMO states forms the excited state [NC]* (step d) which gives rise to the ECL. Adsorption on electrode surface of the reduced NCs intermediates, which carries fewer overall charges and thus are more likely than the highly charged ones, will be better available after the electrode potential is stepped toward positive, and account for the much higher oxidative ECL.

In some instances, the sample for testing can be a buffer solution, a biological sample, or a combination of both. Exemplary buffer solutions include, but are not limited to, phosphate buffer solution (PBS), salt water, MES buffer, Bis-Tris buffer, ADA, ACES, PIPES, MOPSO, Bis-Tris propane, BES, MOPS, TES, HEPES, DIPSO, MOBS, TAPSO, Trizma, HEPPSO, POPSO, TEA, EPPS, Tricine, Gly-gly, Bicine, HEPBS, TAPS, AMPD, TABS, AMPSO, CHES, CAPSO, AMP, CAPS, CABS, or combinations thereof. The buffer solution can have a pH between 3 and 8.5. In some instances, the buffer solution has a pH of 7.4. Exemplary biological samples include bodily fluids or mucus such as saliva, sputum, tear, sweat, urine, exudate, blood, serum, plasma, and vaginal discharge. The analytes can be drugs, biomarkers, metabolites, metal ions, or combinations thereof.

In some instances, the analytes are drugs. In some instances, the analytes are piperazine derivative drugs. Exemplary piperazine derivative drugs include, but are not limited to, HEPES, clozapine, olanzapine, perphenazine, trazodone, and N-substituted piperazines such as morphine, 1-benzylpiperazine.

In some instances, the ECL sensors can be used to determine drug abuse in a subject. In some instances, the ECL sensors include metal nanoclusters assembled on the surface of a conductive substrate. The sample containing piperazine derivative drugs are brought in contact with the ECL sensors. Upon applying a potential by stepping between a first potential and a second potential, the corresponding energy states of the metal nanoclusters and the piperazine derivative drugs are activated. The electron transfer reaction between the activated metal nanoclusters and the piperazine derivative drugs increases the ECL of the metal nanoclusters as compared to the ECL of the metal nanoclusters in the absence of the piperazine derivative drugs. The level of increase of the ECL of the metal nanoclusters is correlated to the concentration of the piperazine derivative drugs. In some instances, an ECL sensing array including two or more ECL sensors can be used to screen a plurality of different piperazine derivative drugs by applying different potentials for each ECL sensor. In some instances, when the analyte of interest can increase the ECL of the metal nanoclusters as compared to the ECL of the metal nanoclusters in the absence of the analyte, the ECL sensor does not include any additional coreactants.

In some instances, the analytes can be metal ions such as Ca²⁺, Mg²⁺, Zn²⁺, Fe²⁺, and Fe³⁺. In some instances, the ECL sensors include metal nanoclusters and coreactants. The coreactants can be non-covalently associated with the metal nanoclusters or in close proximity to the metal nanoclusters upon placement in a sample. In some instances, the metal ions and coreactants can be assembled on the surface of a conductive substrate. In some instances, the metal ions and coreactants are not assembled on the surface of a conductive substrate. In some instances, the sample containing metal ions are brought in contact with the ECL sensors. In some instances, the metal ions can interact with the metal nanoclusters and/or the coreactants through non-covalent interactions. Upon applying a potential by stepping between a first potential and a second potential, the corresponding energy states of the metal nanoclusters and the coreactants are activated. As a result, the ECL of the metal nanoclusters increases or decreases as compared to the metal nanoclusters in the absence of the metal ions. The level of increase or decrease of the ECL of the metal nanoclusters is correlated to the concentration of the metal ions. In some instances, an ECL sensing array including two or more ECL sensors can be used to screen a plurality of different metal ions based on the different interactions between the metal ions and the metal nanoclusters and/or the coreactants.

In some instances, the sample volume for testing can be between about 0.1 μL and about 1 mL. In some instances, the volume of test sample can be between about 0.1 μL and about 100 μL, between about 0.1 μL and about 50 μL, between about 0.1 μL and about 30 μL, between about 1 μL and about 30 μL, or between about 10 μL and about 30 μL. In some instances, the sample volume for testing is about 30 μL.

The ECL signal can be measured by any suitable means for photon detection. In some instances, the ECL of the metal nanoclusters can be detected by a camera. In some instances, an acquisition system, including, for example, a potentiostat or a power supply to provide a potential to the ECL sensors coupled with a detection component such as a camera, can be used to detect the ECL of the ECL sensors. In some instances, the acquisition system can be connected to a display component. In some instances, the display component can provide for electronic conversion, such as via software, to convert the signals received from the acquisition system to a concentration value or a graph, which is then displayed on the screen. Such electronic conversions are known in the art.

### V. Kits

Kits containing the ECL sensors or ECL sensing arrays for testing samples, and, optionally, one or more containers with buffers for preparing the samples for detection are also described herein. The kits can also include an instruction manual for sampling and detection of the one or more analytes. Kits can also include instructions on instrument and/or software settings for calibrating and detecting the analyte concentration.

The disclosed ECL sensors and methods can be further understood through the following numbered paragraphs.

1. An electrochemiluminescence sensor comprising metal nanoclusters.

2. The electrochemiluminescence sensor of paragraph 1, wherein the metal nanoclusters are organo-soluble or aqueous soluble.

3. The electrochemiluminescence sensor of paragraph 1 or paragraph 2, wherein the metal nanocluster comprises a metal core and a plurality of ligands.

4. The electrochemiluminescence sensor of paragraph 3, wherein the metal core comprises metal atoms of the same type or a mixture of metal atoms of different types.

5. The electrochemiluminescence sensor of paragraph 3 or paragraph 4, wherein the ligands comprise thiolates, phosphines, halogens, or combinations thereof.

6. The electrochemiluminescence sensor of paragraph 4 or paragraph 5, wherein the metal atoms are selected from the group consisting of gold, silver, aluminum, tin, magnesium, copper, nickel, iron, cobalt, magnesium, platinum, palladium, iridium, vanadium, rhodium, and ruthenium.

7. The electrochemiluminescence sensor of any one of paragraphs 4-6, wherein the metal atoms are gold.

8. The electrochemiluminescence sensor of any one of paragraphs 4-6, wherein the mixture of metal atoms contains gold and silver.

9. The electrochemiluminescence sensor of any one of paragraphs 3-8, wherein the metal nanoclusters further comprise targeting moieties bound to the core, to the ligands, or to both the core and the ligands of the metal nanoclusters.

10. The electrochemiluminescence sensor of any one of paragraphs 1-9 further comprising a conductive substrate.

11. The electrochemiluminescence sensor of paragraph 10, wherein the metal nanoclusters are assembled on the surface of the conductive substrate.

12. The electrochemiluminescence sensor of any one of paragraphs 1-11 further comprising a coreactant.

13. The electrochemiluminescence sensor of paragraph 12, wherein the coreactant is associated with the metal nanoclusters covalently or non-covalently.

14. The electrochemiluminescence sensor of paragraph 12 or paragraph 13, wherein the coreactant is selected from the group consisting of amines, oxalates, persulfates, hydrogen peroxide, nitrile, unsubstituted cyano, substituted cyano, unsubstituted benzophenone, substituted benzophenone, unsubstituted benzoic acid, substituted benzoic acid, unsubstituted naphthalene, substituted naphthalene, unsubstituted biphenyl, and substituted biphenyl.

15. The electrochemiluminescence sensor of any one of paragraphs 12-14, wherein the coreactant is an amine.

16. The electrochemiluminescence sensor of any one of paragraph 12-15, wherein the coreactant is a tertiary amine.

17. The electrochemiluminescence sensor of any one of paragraphs 1-16, wherein the metal nanoclusters display near-IR electrochemiluminescence.

18. The electrochemiluminescence sensor of any one of paragraphs 1-17, wherein the metal nanoclusters display electrochemiluminescence higher than tris(bipyridine)ruthenium(II) complex under the same conditions.

19. The electrochemiluminescence sensor of any one of paragraphs 1-18, wherein the metal nanoclusters display electrochemiluminescence that is at least 2 times, at least 5 times, at least 10 times, at least 20 times, at least 25 times, at least 30 times, at least 50 times, at least 100 times, at least 150 times, at least 200 times, at least 250 times, at least 300 times, at least 350 times, or at least 400 times higher than tris(bipyridine)ruthenium(II) complex under the same conditions.

20. The electrochemiluminescence sensor of any one of paragraphs 1-19, wherein the metal nanoclusters are rod-shaped.

21. An electrochemiluminescence sensing array comprising two or more of the electrochemiluminescence sensors of any one of paragraphs 1-20.

22. A method of testing the presence, absence, or concentration of an analyte of interest in a sample, the method comprising:

(i) contacting the sample with the electrochemiluminescence sensor of any one of paragraphs 1-20,

(ii) applying a potential to the sensor, and

(iii) detecting the electrochemiluminescence and/or a redox current of the metal nanoclusters.

23. A method of screening the presence, absence, or concentration of a plurality of analytes of interest in a sample, the method comprising:

(i) contacting the sample with the electrochemiluminescence sensor array of paragraph 21,

(ii) applying a potential to the sensor, and

(iii) detecting the electrochemiluminescence and/or redox currents of the metal nanoclusters.

24. The method of paragraph 23, wherein the potential applied is the same or different for each of the electrochemiluminescence sensors.

25. The method of any one of paragraphs 22-24, wherein the potential is applied by linear sweeping from a first potential to a second potential, cyclic sweeping between a first potential and a second potential, or stepping between a first potential to a second potential.

26. The method of any one of paragraphs 22-25, wherein the potential is sufficient to provide enough energy to activate the corresponding energy states of the metal nanoclusters, the coreactant, the analyte, or combinations thereof.

27. The method of any one of paragraphs 22-26, wherein the analyte interacts with the metal nanoclusters and/or the coreactant.

28. The method of any one of paragraphs 22-27, wherein the electrochemiluminescence of the metal nanoclusters increases or decreases upon an interaction between the analyte and the metal nanoclusters and/or the coreactant as compared to the electrochemiluminescence of the metal nanoclusters in the absence of the analyte.

29. The method of paragraph 28, wherein the level of increase or decrease of the electrochemiluminescence of the metal nanoclusters is correlated to the concentration of the analyte.

30. The method of any one of paragraphs 22-29, wherein the redox current of the metal nanoclusters increases or decreases upon an interaction between the analyte and the metal nanoclusters and/or the coreactant as compared to the redox current of the metal nanoclusters in the absence of the analyte.

31. The method of paragraph 30, wherein the level of increase or decrease of the redox current of the metal nanoclusters is correlated to the concentration of the analyte.

32. The method of any one of paragraphs 22-31, wherein the sample is a buffer solution, a biological sample, or a combination of both.

33. The method of any one of paragraphs 22-32, wherein the sample is a biological sample, wherein the biological sample is a bodily fluid or mucus selected from the group consisting of saliva, sputum, tear, sweat, urine, exudate, blood, serum, plasma, and vaginal discharge.

34. The method of any one of paragraphs 22-33, wherein the analyte is a drug, metabolite, biomarker, metal ion, or combinations thereof.

35. The method of any one of paragraphs 22-34, wherein the analyte is a piperazine derivative drug.

36. The method of any one of paragraphs 22-35, wherein the electrochemiluminescence of the metal nanoclusters is detected by a camera.

The present invention will be further understood by reference to the following non-limiting examples.

### EXAMPLES

### Example 1. Synthesis and Characterization of Metal Nanoclusters

Materials and Methods

Materials

Tetrachloroauric(III) acid (HAuCl₄.3H₂O, >99.99% metals basis), Sodium borohydride (NaBH₄, >98%), and Triethyl-amine (Et₃N, 99%) were received from ACROS Organic. Triphenylphosphine (PPh₃, 99%), 2-Phenylethanethiol (HSC₂H₄Ph, 98%), Tetraoctylammonium bromide (TOABr, 98%), Tris(2,2′-bipyridyl)dichlororuthenium(II) hexahy-drate (Ru(bpy)₃, 99.95%), Tripropylamine (TPrA, ≥98%), Toluene (TOL, HPLC grade, ≥99.9%), Acetonitrile (ACN, HPLC grade, ≥99.9%), Ethanol (EtOH, HPLC grade, ≥99.9%), Hexane (Hex, HPLC grade, ≥99.9%), Methylene chloride (DCM, HPLC grade, ≥99.9%), Sodium hex-afluoroantimonate (NaSbF₆, technical grade) were from Sigma-Aldrich. In all experiments, nanopure water (>18 MΩ·cm) from a Barnstead system was used.

Synthesis of Ag(I)-Thiolate Complex

Ag(I)-SPhC₂H₄ compound was prepared following a reported procedure (Li, et al., J. Am. Chem. Soc., 132:17678-17679 (2010)). Briefly, 1 g AgNO₃ (5.89 mmol) was first dissolved in a mixed solvent of 1 ml H₂O and 5 ml EtOH under ultrasonication. Then a mixture of 2 ml Et₃N containing 5.65 mmol 2-phenylethanethiol (0.78 g) was added dropwise over 10 min under stirring forming a white turbid solution. The reaction proceeded for 30 minutes at room temperature. After centrifugation, the crude precipitate was washed with EtOH several times. The final white product [Ag(I)-thiolate] complex was collected after the removal of solvent under vacuum at room temperature.

Synthesis of [Au₁₁(PPh₃)₈Cl₂]⁺

The synthesis of triphenylphosphine-stabilized Au clusters followed the recipe described in literature (Vollenbroek, et al., Inorg. Chem., 17:1345 (1978)). An aqueous solution of HAuCl₄.3H₂O (0.4 ml, 0.2 g/mL) was dissolved in 10 ml of EtOH, and then solid triphenylphosphine (0.16 g, 0.293 mmol) was added. The solution turned into a white suspension in 1 min. a fresh solution of NaBH₄ (0.019 g, 0.48 mmol, dissolved in 5 mL EtOH) was added to the solution. After ˜2 hours, the reaction mixture containing monodisperse Au₁₁ clusters was obtained. The crude product was purified with DCM/Hex solvents.

Synthesis of Phosphine-Protected Polydispersed Au Nanoparticles

Au nanoparticles capped with triphenylphosphine (Au—PPh₃ nanoparticles) were prepared based on the recipe in the reference (Qian, et al., Inorg. Chem., 50(21):10732-10739 (2011)). HAuCl₄.3H₂O (0.16 g, 0.4 mmol, dissolved in 5 ml H₂O) and TOABr (0.254 g, 0.465 mmol, dissolved in 10 ml TOL) were mixed and stirred vigorously for 15 min to complete phase transfer process. After the aqueous phase was removed, 0.313 g (1.2 mmol) PPh₃ was added to the TOL solution under moderate stirring. After ˜3 minutes, NaBH₄ (0.070 g, 1.85 mmol, dissolved in 5 ml EtOH) was injected to the mixture. The solution immediately became dark. The reaction was allowed to proceed for 70 minutes at room temperature. The black products were washed several times with hexane and collected by rotary evaporation.

Synthesis of [AgₓAu₂₅₋ₓ(PPh₃)₁₀(SC₂H₄Ph)₅Cl₂]²⁺ (x≤12) nanoclusters [AgₓAu₂₅₋ₓ(PPh₃)₁₀(SC₂H₄Ph)₅Cl₂]²⁺ (x≤12) nanoclusters were synthesized with Au—PPh₃ nanoparticles and Ag(I)—SC₂H₄Ph compounds as precursors. Specifically, PhC₂H₄S—Ag (80 mg) and Au—PPh₃ nanoparticles (75 mg) were added to the ethanol solution (10 mL) under vigorous stirring at 298 K. After 6 h, the product was dried in vacuum, washed several times with ethanol/hexane (1:3, V/V), a mixed solvent of DCM and Hex was used to extract clusters. The brownish-yellow solution was collected and mixed with an excess of NaSbF₆. Insoluble products were collected on a filter and crystallized from a mixed solution of DCM and diethyl ether.

Synthesis of Au₂₅(SC₂H₄ pH)₁₈⁻ Nanoclusters

Au₂₅(SC₂H₄Ph)₁₈⁻ is nanoclusters were prepared following the reported procedure in reference (Zhu, et al., J. Am. Chem. Soc., 130:1138 (2008)). Specifically, HAuCl₄.3H₂O (0.1576 g, 0.4 mmol) dissolved in 5 mL nanopure water, and TOABr (0.2558 g, 0.47 mmol) dissolved in 10 mL toluene were added in a 25 mL tri-neck round bottom flask under vigorous stirring. After 15 min for the phase transfer to complete, the aqueous layer was removed, and the toluene solution of Au(III) was cooled down to 0° C. in an ice bath over a period of 30 min under magnetic stirring. PhC₂H₄SH (0.17 mL) was added; stirring was reduced to a very low speed (˜30 rpm) in 1 hour. A fresh aqueous solution of NaBH₄ (0.1550 g in 7 mL ice-cold water) was quickly added. The reaction was allowed to proceed overnight. EtOH and ACN were used to wash the products and extract the Au₂₅(SC₂H₄Ph)₁₈⁻ is respectively.

Synthesis of [AgₓAu₂₅₋ₓ(PPh₃)₁₀(SR)₅Cl₂]²⁺ (x≤13) Nanoclusters

[AgₓAu₂₅₋ₓ(PPh₃)₁₀(SR)₅Cl₂]²⁺ (x≤13) nanoclusters were synthesized by the reaction of [Au₁₁(PPh₃)₈Cl₂]⁺ clusters with Ag(I)-SPhC₂H₄ complexes. Briefly, Ag(I)—SPhC₂H₄ (80 mg, 0.33 mmol) was added to an Au₁₁ NCs (75 mg, 0.017 mmol) EtOH solution (10 mL) under vigorous stirring at 313 K. After 6 h, the product was dried under vacuum and washed several times with EtOH/Hex (1:3, V/V). NaSbF₆ was used to substitute the anions in the clusters for recrystallization. The nanoclusters crystals were redissolved for analysis.

Characterization of Au₁₂Ag₁₁

UV-visible spectra were recorded using a Shimadzu UV-1700 spectrophotometer. Fluorescence spectra were measured with a Horiba Jobin-Yvon Fluorolog 311 spectrometer equipped with PMT visible and InGaAs near-IR detectors.

Results

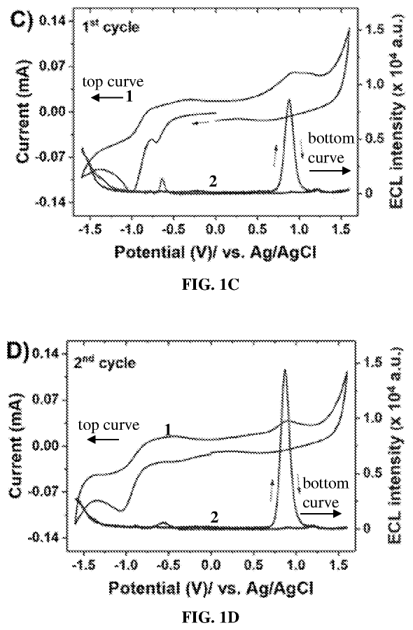

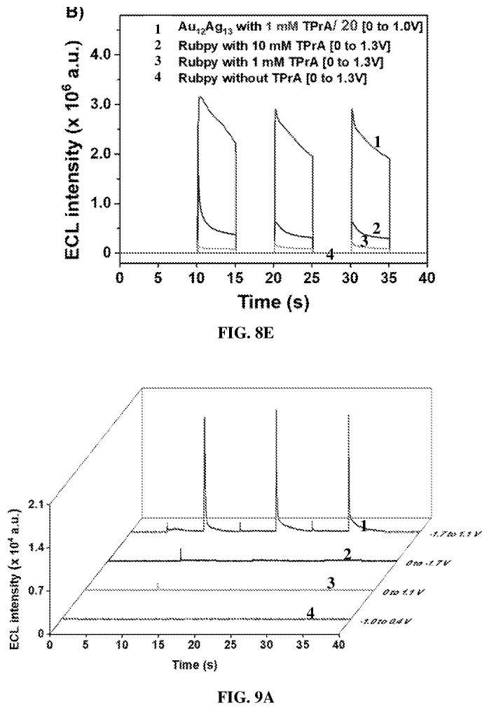

The UV-visible spectrum of Au₁₂Ag₁₃ in 1:1 TOL/ACN shows multiple absorption peaks, demonstrating the successful synthesis and purification of Au₁₂Ag₁₃ (FIG. 11) (Wang, et al., Angew. Chem. Int. Ed., 53(9):2376-80 (2014)). The discrete absorption bands corresponding to the electronic transitions of the related energy states of this Au₁₂Ag₁₃ nanocluster. The photon energy spectrum of Au₁₂Ag₁₃ in 1:1 TOL/ACN, which is a replot of the data in FIG. 11, shows the photon energy features of Au₁₂Ag₁₃ (FIG. 2C) (Wang, et al., Angew. Chem. Int. Ed., 53(9):2376-80 (2014)).

### Example 2. Metal Nanoclusters Generate Strong Self-Annihilation ECL without Coreactant

Materials and Methods

Voltammetry Measurements of Au₁₂Ag₁₃

Voltammetry was recorded with a CHI Instrument (Model 750C). In general, the nanoclusters were dissolved in 50%:50% toluene:acetonitrile at 1 mM concentration with 0.1 M TBAP (tetra-n-butylammonium perchlorate) supporting electrolyte. A Pt disk electrode (d 0.5 mm) was used as working electrode, while an Ag/AgCl wire and a Pt foil were used as reference and counter electrodes respectively. A 20 mM purging with Argon was executed before each measurement.

ECL Measurements

ECL experiments were performed with a three-electrode system in a quartz cuvette. A Pt mesh working electrode and a cuvette were aligned at a fixed position with respect to the camera for consistency. An Ag/AgCl wire as reference electrode and a Pt foil as counter electrode were used. The emission intensity was recorded with an Andor iDUS CCD camera (Model DU401A-BR-DD). The camera was externally triggered by the potentiostat (Gamry R600) for synchronization. The ECL spectrum was collected with the Fluorolog 311 spectrometer bypassing the excitation. The sample solution was purged for about 20 mM with Argon prior to the measurements.

Results

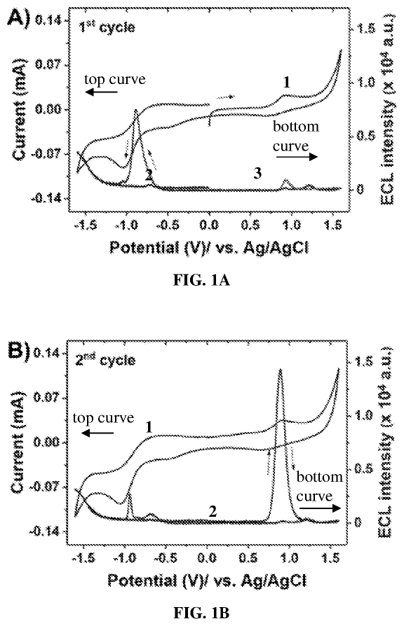

The self-annihilation ECL of Au₁₂Ag₁₃ rod without coreactant was firstly studied by cyclic voltammetry (CV) shown in FIGS. 1A-1D. Both oxidative and reductive ECL were observed, with onset potential for the major ECL peaks at +0.75 and −0.66, and peak potentials at +0.87 and −0.90 respectively. The ECL signals correlate with the currents on both the oxidation and reduction sides well. Two consecutive CV/ECL cycles were provided with opposite initial scan directions to highlight the ECL dependence on the initial/preexisting conditions. In the first cycles shown in FIGS. 1A and 1C, neither oxidative nor reductive ECL was apparent in the first segment (curve 3 in FIGS. 1A and 1C, starting from 0V to positive and negative respectively). Both the oxidative and reductive ECL appeared in the reversal scan. In other words, the reductive ECL was only observed after anodic species were produced by the electrode oxidation process, and vice versa. The CV/ECL profiles converged in second cycle, i.e., the largely similar curves in FIGS. 1B and 1D, and ultimately the CV/ECL curves overlapped in later cycles (not shown). These ECL features correspond to the classic self-annihilation pathways requiring both cathodic and anodic radical intermediates generated by the electrode reactions. Without either radical intermediate, no major ECL was detected (first segment in FIGS. 1A and 1C).

Importantly, the major reductive ECL peak in the scan to negative potentials in FIG. 1A diminished in later scans (FIGS. 1B and 1D), while the oxidative ECL got higher. The changes show that the radical intermediates by reduction were more accessible to react with the ones generated by oxidation, while the oxidized ones became unavailable to sustain the cathodic ECL. There were minor ECL features corresponding to either the small amount of impurities (other nanoclusters Ag<13 that cannot be separated so far) or the products of the radical intermediates, or both.

To better understand the ECL features, main redox activities were analyzed by differential pulse voltammetry (DPV) and CV using a more concentrated sample shown in FIGS. 2A and 2B. A pair of anodic peaks has comparable current amplitudes with the first cathodic peak in CV. The peak spacing is about 0.26 V between the pair of oxidation peaks at O1 (˜0.820 V), O2 (˜1.084 V), which corresponds to a charging energy of 0.26 eV. In reference to the electrochemical properties of similar nanoclusters such as Au₂₅, these are assigned to 2 e⁻ oxidation at HOMO and 1 e⁻ reduction to LUMO (Park, et al., Langmuir, 28(17):7049-7054 (2012)). An electrochemical HOMO-LUMO energy gap of 1.55 eV can be determined from the difference between the first oxidation (O1) and first reduction (R1, about −0.996 V) potentials after charging energy subtraction, which matches the optical gap of 1.56 eV (See FIG. 2C). The O3 (˜1.456 V) peak corresponds to the states below HOMO, which are more negative than the homometallic Au₂₅ rod. Another prominent cathodic peak (R3, about −1.416 V) can be seen in CVs, while a small shoulder can be seen (R2, about −1.292 V) in DPV. The much lower current of R2 and R3 in CV shows less stability after RE To mitigate the impacts of irreversible decomposition of the nanoclusters, in the following potential step experiments, only the first oxidation/reduction peak was invoked for ECL generation to gain mechanistic and quantitative understanding.

### Example 3. Metal Nanoclusters Generate Extremely High Transient ECL in Potential-Step Experiments

Materials and Methods

The potential on the working electrode was stepped between −1.2 V and +1.0 V cyclically every 5 s, which covered the first reduction peak and first oxidation peak. Reduced and oxidized radical intermediates were generated within the same electrode-solution interface consecutively.

Results

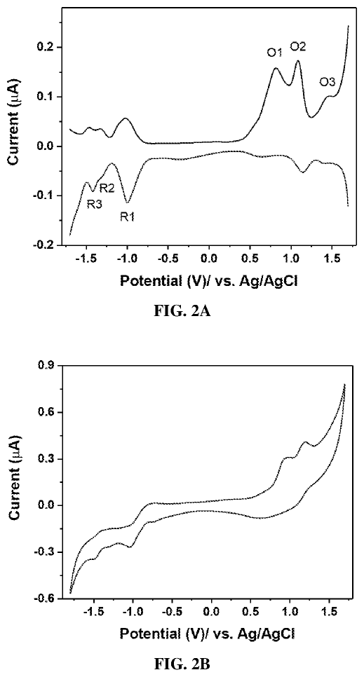

As shown in FIG. 3A, a surprisingly high oxidative ECL signal was observed under +1.0 V (onset 10, 20 and 30 s). This self-annihilation ECL is orders magnitudes higher than other gold nanoclusters tested so far in aqueous and organic solvents (Hesari, et al., Acc. Chem. Res., 50(2):218-230 (2017); Wang, et al., J. Am. Chem. Soc., 138(20):6380-6383 (2016); Wang, et al., Chem Electro Chem, 4(7):1697-1701 (2017); Fang, et al., Chem. Commun., 47(8):2369-2371 (2011)). FIG. 3B shows the ECL signal in log scale to better illustrate the gradual decay after the transient ECL peak under both positive and negative potentials. The first and last 5 s data provides the baseline, without electrode reactions for comparison. In FIGS. 3C and 3D, the first 0.3 s data was plotted for each anodic and cathodic process separately. About two/three points were captured during the initial transient process. Therefore, the ECL peak intensity, other than the first step at −1.2V, was limited by the camera's temporal resolution at 13.3 ms and could not be compared quantitatively.

Overall, the step ECL profiles include a transient signal (tens milliseconds) with extremely high intensity, followed by a gradual process throughout the applied potential period. Similar to sweeping experiments, oxidative ECL is orders magnitudes higher than the ECL under reduction potential. The oxidative ECL profiles (+1.0 V) were highly consistent. For the weaker reductive ECL, the ECL peak during first step to −1.2 V (onset at 5 s) was absent. This is similar to the first segment in CV/ECL measurements when the oxidized radical intermediate was not available. A second cathodic ECL peak/shoulder can be seen in the 2nd/3rd cycles in FIG. 3D, which was reminiscent of diffusion induced concentration profiles in classic CV analysis as well as the diffusive annihilation ECL of Rubpy (Lee, et al., ACS Appl. Mater. Interfaces, 10(48):41562-41569 (2018)). Both anodic and cathodic ECL profiles stabilized, i.e., displaying similar shapes qualitatively, toward later cycles because of the establishment of both radical intermediates at ‘steady-state’ in the diffuse layer.

Two pathways can account for these ECL features. The first is diffusion reactions of the radical intermediates, i.e., homogeneous electron transfers (ETs) among the redox species in solution. The gradual decay arose primarily from this process. Heterogeneous ETs of surface adsorbed species after the potential steps are proposed as the other pathway to explain the transient signal.

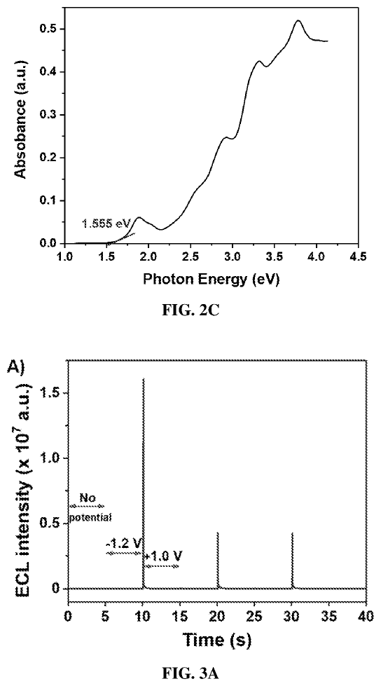

To evaluate the differences in the reduction and oxidation ECL features, electrolysis was performed to accumulate the possible surface-adsorbed intermediates and thus to amplify the impacts. The CVs before and after the electrolysis under reduction (−1.2 V) are shown in FIG. 4, clearly displaying a new redox process at about +0.7 V with both anodic and cathodic current features. Although not perfectly symmetric, the anodic and cathodic peaks at the same potential is characteristic of surface adsorbed species rather than diffusion processes. Further, the new anodic peak at about +0.7 V decreased in later cycles, corresponding to the loss of surface deposited species from reductive electrolysis. Note the potential was limited within positive potential range (curve 1 and curve 2) to avoid additional reduction. By limiting the potential to the negative range without inducing further oxidation after a separate electrolysis at about −1.2 V, the redox features are slightly better defined such as a small peak about −0.7V (in reference to FIG. 2B) but no new feature as prominent as the +0.7V ones emerged. The broad decrease in the anodic current (curve 3 vs. curve 4) shows that those adsorbed intermediates after reduction could not be oxidized until the potential was more positive than about +0.7 V. The observation also explains the diminishment of the reductive ECL in later cycles (See FIG. 1A).

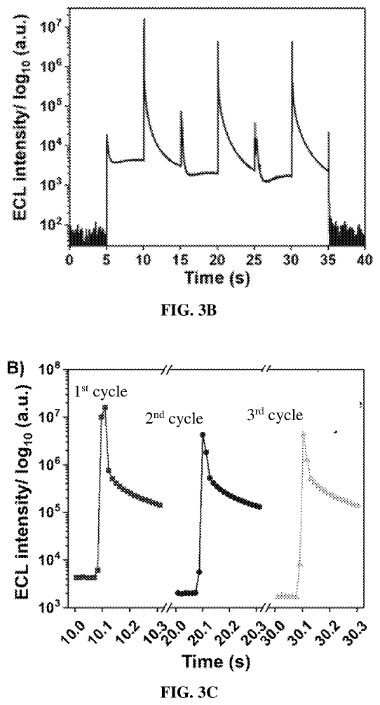

Those CV comparisons after positive electrolysis are shown in FIG. 5. No major difference was detected after the electrolysis at +1.1 V, which shows that the oxidized intermediate species are diffusive rather than surface adsorbed. Correspondingly, the anodic intermediates are less available to sustain the reductive ECL especially the transient signal. The surface adsorption features strongly support the proposed mechanism that the reduced intermediates were more accessible to react with the ones generated by oxidation, and generating higher oxidative ECL.

### Example 4. Pathway of ECL Generation by Metal Nanoclusters

Materials and Methods

The potential on the working electrode was stepped cyclically every 5 s between −1.2 V and +1.0 V, between +0.5 V and −0.5 V, between 0 V and +1 V, or between 0 V and −1.2 V.

Results

Scheme of the ECL reaction pathways:

| | |
|---|---|
| [NCs] + e⁻ → [NCs]^{-•} | (a) |
| [NCs]^{-•} − e⁻ → [NCs]^{*} | (b) |
| [NCs] − e⁻ → [NCs]^{+•} | (c) |
| [NCs]^{+•} + e⁻ → [NCs]^{*} | (d) |
| [NCs]^{-•} + [NCs]^{+•} → [NCs]^{*} + [NCs] | (e) |
| [NCs]^{*} → [NCs] + hv | (f) |

The involved energy states in the ECL reactions are shown in FIGS. 6A and 6B. In steps a and c, the reduced and oxidized species are generated by electrode ET reactions. Step e is the diffusive reaction between the two types of radical intermediates, after which the excited [NC]* releases the energy via photon emission (step f). This mechanism is the classic self-annihilation pathway (Miao, Chem. Rev., 108(7):2506-2553 (2008)). The mechanism for the transient oxidative ECL peak is shown in FIG. 6A. An electron is firstly injected into the LUMO of Au₁₂Ag₁₃ (step a). Those cathodically produced radicals are oxidized once the electrode potential is stepped below HOMO (step b). Loss of the HOMO electron directly produces the excited species [NC]* which relaxes to the ground state and emits light. This pathway only involves those pre-reduced NCs directly at the electrode surface vicinity. Further, loss of the electron in LUMO after step a, however, will quench the emission process. Therefore, the ECL displays a transient peak which quickly decays within milli-seconds. Similarly, FIG. 6B shows the reductive ECL processes (onset 5, 15 and 25 s). Loss of the HOMO electron (step c) followed by the reduction to LUMO states forms the excited state [NC]* (step d) which gives rise to the ECL. Impurities absorbed on the electrode surface, such as nanoclusters containing Ag ≤12, can have different energy states thus may serve as electron donors, which contribute to ECL signals that are much weaker than Au₁₂Ag₁₃. Adsorption of the reduced NCs intermediates on the electrode surface, which carries fewer overall charges and thus are more likely than the highly charged ones, will be better available after the electrode potential is stepped toward positive, and account for the much higher oxidative ECL.

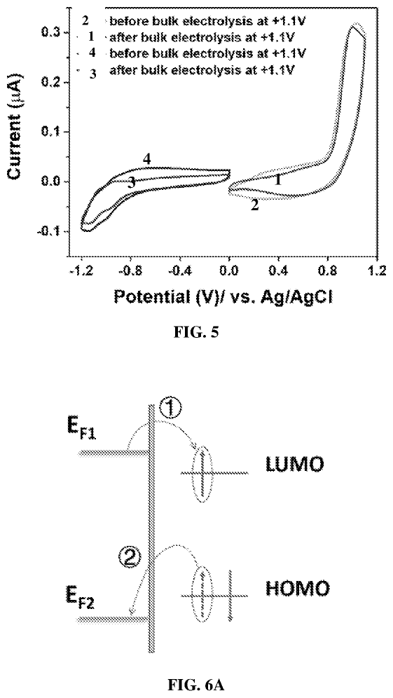

FIG. 7 shows important controls to further support the proposed mechanism of self-annihilation ECL. No ECL was detected when the electrode potential was stepped between ±0.5V because no redox reactions occurred to generate the radical species (curve 1). Weak ECL, orders magnitudes lower than the HOMO-LUMO ECL, was observed under the oxidation only (0V-+1V) and reduction only (0V-−1.2V) conditions. Similar to the small ECL peaks in the CV/ECL results shown in FIGS. 1A-1D, the weak signals are attributed to the reaction of the oxidized/reduced species with other nanoclusters/impurities. Within each ECL cycle, a fast decay of ECL intensity was observed which can be explained by the simple diffusion induced concentration profiles away from the electrode-solution interface.

### Example 5. ECL Generated by Metal Nanoclusters Shows Much Higher and Long-Lasting Signal Compared with Ru(Bpy)₃

Materials and Methods

The ECL efficiency was assessed by comparing to Ru(bpy)₃-only and Ru(bpy)₃-TPrA under the same measurement conditions (Miao, et al., J. Am. Chem. Soc., 124:14478-14485 (2002)).

Results

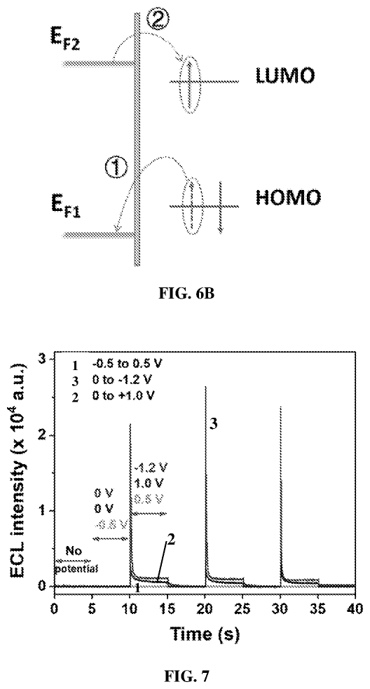

FIG. 8A shows the self-annihilation ECL profiles excited at their respective HOMO-LUMO redox potentials. The ECL intensity of Au₁₂Ag₁₃ is about ten times higher than that from Ru(bpy)₃-only compared by the integrated peak area. FIG. 8B displays the ECL intensity in log scale to show the gradual decay profile. The potential steps for Ru(bpy)₃ (−1.3 to 1.3 V) were based on the CV of Ru(bpy)₃ in the test solvent/electrolyte (See FIG. 8C, curve 1). The CV/ECL graph of Ru(bpy)₃-only is shown in FIG. 8D, which also confirms the much higher self-annihilation of Au₁₂Ag₁₃ over Ru(bpy)₃ itself.

The coreactant ECL was also evaluated in reference to the standard Ru(bpy)₃-TPrA system. As shown in FIG. 8E, the ECL signal of Au₁₂Ag₁₃ with 1 mM TPrA, which was reduced by 20-fold to fit in the same graph, was still much higher than Rubpy with 1 mM and 10 mM TPrA, respectively. Ru(bpy)₃ ECL with two TPrA concentrations are presented as a better calibration/reference. Comparing the same coreactant concentration at 1 mM, the ECL of Au₁₂Ag₁₃ (integrated area) is about 400 times higher, which is a statistically significant difference. The ECL intensity of Au₁₂Ag₁₃ showed a more gradual signal attenuation than Ru(bpy)₃ in coreactant measurements. The long lasting ECL signal is highly favorable for use of Au₁₂Ag₁₃ in ECL applications.

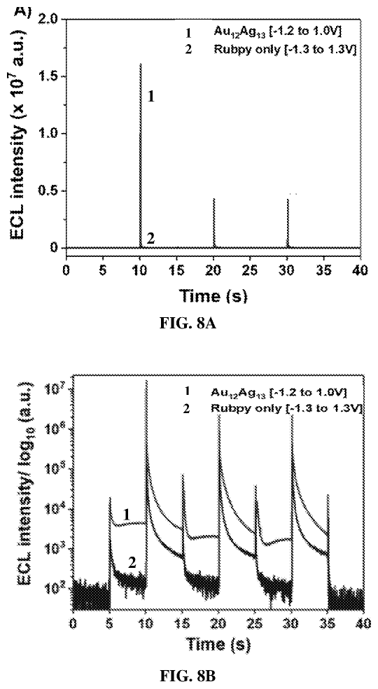

Further, the [AgₓAu₂₅₋ₓ(PPh₃)₁₀(SR)₅Cl₂]²⁺ (with Ag less than 13, x≤12) nanoclusters and prototype spherical Au₂₅(SC₂H₄Ph)₁₈⁻ nanoclusters were measured under comparable conditions as controls. Their step-ECL profiles are shown in FIGS. 9A and 9B, respectively. Both show orders magnitudes weaker ECL compared to Ag₁₃Au₁₂. The only appreciable ECL signals were recorded after the excitation of both HOMO and LUMO state via electron transfer reactions.

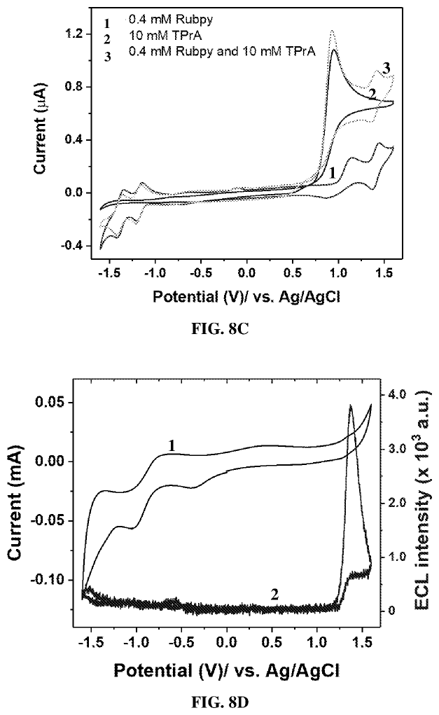

The ECL spectrum was compared with photoluminescence (PL) spectrum in FIG. 10. The ECL is clearly in the near-IR range, with a peak around 775 nm that is slightly redshifted (10 nm) compared to the PL.

The Examples have demonstrated unexpectedly intense near-IR ECL generated from an exemplary bimetal Ag₁₃Au₁₂ nanocluster. The self-annihilation ECL was found to be much higher via reductive-oxidation pathways. The observation is explained by the adsorption of LUMO-reduced NCs on electrode surfaces, which were captured by cyclic voltammetry measurements. Potential-step measurements revealed an extremely high and transient (milliseconds) ECL upon HOMO oxidation at +1.0 V after the LUMO-reduction, followed by a gradual decay under the applied constant potential. The pathways for the cathodic and anodic annihilation ECL are based on the basic energy diagram determined from the main redox features. The ECL intensity from Au₁₂Ag₁₃ is about ten times higher than that from Ru(bpy)₃ when applying the potential to activate their respective HOMO-LUMO states. With 1 mM TPrA as a coreactant, the ECL of Au₁₂Ag₁₃ is about 400 times higher than Ru(bpy)₃, the ECL standard since its establishment. The strong ECL of Au₁₂Ag₁₃ can be attributed to the 13th Ag atom at the central position. Without being bound to a particular theory of operation, this central Ag atom appears to stabilize the charges on LUMO orbital and makes the rod-shape Ag₁₃Au₁₂ core more rigid. Thus, combinations of metal atoms that produce similar stability can also produce higher ECL. Such metal nanoclusters with unexpected high ECL provide new tools in applications such as sensing and assay analysis.

### Example 6. Metal Nanoclusters Assembled on Electrode Surface Generates Near-IR ECL

Materials and Methods

Materials

[2-[4-[(4-Chlorophenyl)phenylmethyl]-1-piperazinyl]ethoxy]acetic acid dihydrochloride (Cetirizine, ≥98%), sodium perchlorate hydrate (NaClO₄.xH₂O, ≥99.99%), sodium phosphate monobasic monohydrate (NaH₂PO₄H₂O, ≥98%), sodium phosphate dibasic heptahydrate (NaH₂PO₄.7H₂O, ≥98%), chloroform (CHCl₃, ≥99.8%), were purchased from Sigma-Aldrich and used as received. Methylene chloride or dichloromethane (CH₂Cl₂, HPLC grade), acetonitrile (CH₃CN, HPLC grade) were purchased from Fisher chemical and dried before used. Rod-shape [AgₓAu₂₅₋ₓ(PPh₃)₁₀(SC₂H₄Ph)₅Cl₂]²⁺ (x≤13) NCs were provided by Dr. Manzhou Zhu's group (Wang, et al., Angewandte Chemie International Edition, 53(9):2376-2380 (2014)). In all aqueous solution preparations, nanopure water (>18 MΩcm) from a Barnstead system was used.

A stock solution of [Ag₁₃Au₁₂(PPh₃)₁₀(SC₂H₄Ph)₅Cl₂]²⁺ NCs dissolved in organic solvent dichloromethane (DCM) was used for surface film preparation. The Ag₁₃Au₁₂ NC film on ITO electrode was insoluble in the aqueous solution and thus was intrinsically stable for analysis applications, for example, biological samples at physiological pH (i.e., about pH 7.4).

ITO Electrode Preparation

Corning alkaline earth boro-aluminosilicate glasses coated with indium tin oxide (ITO) were used as working electrodes because of its optical transparency and electrical conductivity. The ITO electrodes were purchased from Delta Technologies (CB-40IN), Rs=4-10Ω. The as prepared ITO surface was relatively hydrophobic that was compatible with organic soluble NCs. The ITO electrode was first cleaned with a general cleaning process by ultrasonicating in nanopure water, ethanol, and nanopure water (1:3), and nanopure water for at least 15 minutes each before use.

Electrochemical Measurements

Cyclic voltammograms were collected using a potentiostat (Gamry Reference 600) with the sample in a Faraday Cage. A three-electrode setup uses an Ag/AgCl wire as a quasi-reference electrode, platinum (Pt) foil as a counter electrode and ITO as a working electrode. Phosphate Buffered saline (PBS) pH 7.4 was used to prepare cetirizine solutions and as controls. Scan rate was 0.1 V/s in all CV measurements.

Near IR ECL Measurements The ECL was measured in a quartz cuvette. A 3D printed spectrometer cuvette holder was used to hold the cuvette in front of the camera window at a fixed position. An ITO working electrode was fixed with a cap on the top of the cuvette to ensure consistent electrode-camera alignment. For results to be directly relevant to real life application settings, all measurements were performed under ambient conditions without degassing. Unless noted otherwise, the electrode potential was held for 2 s at −0.8 V and then stepped to 1.2 V for 6 s. The emission intensity was recorded with an Andor iDUS CCD camera (Model DU401A-BR-DD). To synchronize the camera response and the electrochemical measurements, the camera was externally triggered by the potentiostat (Gamry Reference 600) at time zero when the potential was applied. The ECL intensity was the sum of photon counts from all pixels; the exposure time is 15 ms for step ECL measurement unless otherwise noted.

Microscopy Imaging

The Ag₁₃Au₁₂ NCs on ITO were characterized by optical imaging and electrochemical measurements. A fluorescence microscope (Olympus IX73) was used for the imaging of Ag₁₃Au₁₂ NCs on ITO. A 377+/−50 nm excitation filter and 647 nm long pass emission filter were used to record photoluminescence with 33 ms exposure time. The excitation light source is a high-power LED light (Excelitas Technology, X-Cite 120 LED Boost).

Dip-Coating Protocol

Dip coating method establishes a simplified Langmuir-Blodgett type surface film preparation. A stock solution of Au₁₂Ag₁₃ NCs dissolved in DCM (absorbance at 360 nm is 0.6 measured with a 1 cm light path) was further diluted into different concentrations as deposition solutions (Wang, et al., Angewandte Chemie International Edition, 53(9):2376-2380 (2014)). After an ITO electrode (around 1 cm in length) was vertically immersed in the deposition solution, the ITO was manually pulled out at a constant speed (around 1 mm/s) and then the extra solution accumulated at the bottom was removed by tapping on a filter paper.

Spin-Coating Protocol

Three types of Au₁₂Ag₁₃ NCs solutions were spin coated to fabricate a NC film on an ITO electrode with a spin coater (Chemat Technology series KW-4A) with 1.0 CG aluminum vacuum chuck under vacuum condition. The first solution was the stock solution in pure DCM with the absorbance at 360 nm at about 0.6 measured with a 1 cm light path. The spin speed was 600 RPM and the time of spin is set to 1 min with dynamic dispense. A 5 μL drop of the NC solution was applied either 10 times or 25 times during the dynamic spinning to deposit different amount of NCs. The second solution was a 1:1 volume ratio of the stock DCM solution and ACN mixture, and the third solution was a 1:1 volume ratio of stock DCM solution with chloroform mixture. Dilution with either ACN or chloroform were made to slow down the evaporation rate for a more uniformed film deposition. During the dynamic spin, the speed was 900 RPM, and the duration was 1 minute.

Results

Metal Nanoclusters Assembled on ITO Electrode Surface by Spin-Coating with a Single Solvent

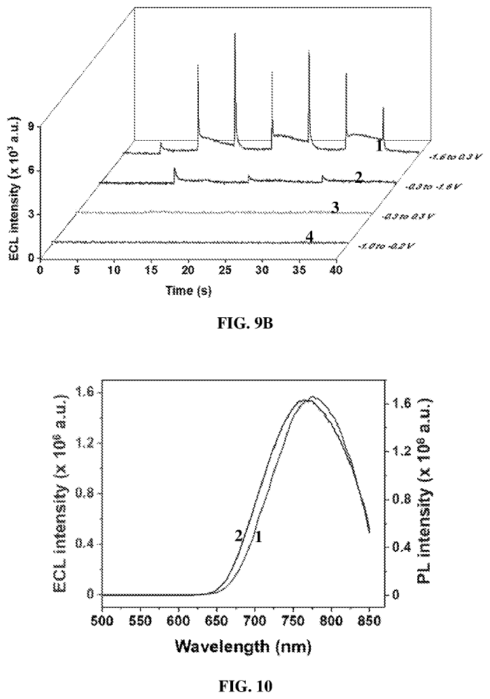

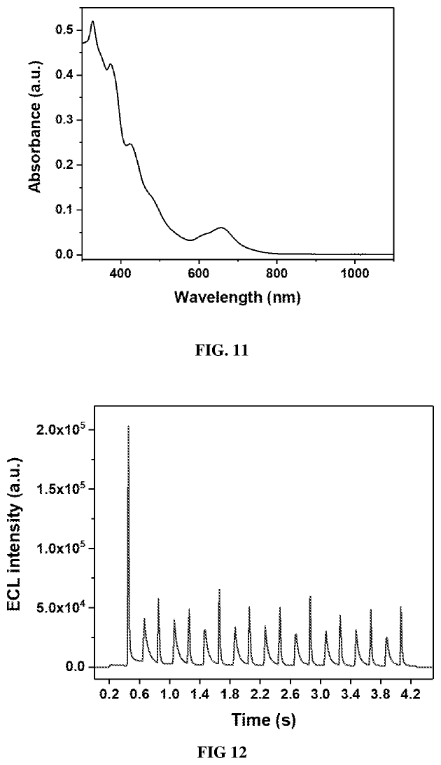

Different surface assembly methods were explored to achieve controlled surface distribution and coverage of NCs on the ITO electrode. A widely distributed and low surface coverage are of fundamental interest toward single molecule type studies, in which the signal is low. A high surface coverage and uniform film is suitable to generate strong and consistent signals for detection applications. The assembly of organic soluble metal NCs on the electrode surface makes them compatible and applicable to applications in aqueous environment. The elimination of the diffusion process involving metal NCs could also simplifies the mechanism and improves the enhancement of near-IR ECL and current signals by target analytes, such as piperazine drugs. ECL responses from surface assembled NCs prepared with different methods are compared (not shown). The results show that more material, i.e., 25 drops over 10 drops in spin-coating, generate stronger ECL signals (about 10 folds stronger). Both spin-coating preparations produce much stronger ECL signal compared to the dip-coating method. Strong ECL from the surface assembled NCs was detected in a PBS buffer at pH 7.4, where the potential was stepped from −1.0 V to 0.9 V with 0.2 s holding time for each potential (1 cycle) and repeated for 10 cycles (see FIG. 12). Further, the surface-based ECL generation can be repeated for at least 240 cycles as shown in FIG. 13. This persistent ECL generation over hundreds cycles demonstrates its potential as sensors in practical applications.

Without coreactants in the test solution, the ECL signal generates from self-annihilation pathway, i.e., both oxidized and reduced NCs are needed to generate the ECL signal. The ECL signal is therefore the strongest when the positive/negative potentials were stepped which decays quickly with each potential step. More specifically, a single oxidation or reduction potential would not generate ECL, as is the case from zero to 0.3 seconds in the first step. Pre-existing reduced/oxidized NCs are necessary (via electrode reduction/oxidation) to react with the oxidized/reduced NCs produced by the subsequently applied potential. The first cycle generates much stronger ECL compared to later cycles regardless the surface preparation methods. Degradation of NCs and irreversible changes of surface assembly structures are among possible reasons for the decreased signal in later cycles.

The near-IR photoluminescence of Au₁₂Ag₁₃ NCs displays an emission maximum at around 760 nm after excited with 365 nm wavelength. The UV-visible absorbance and luminescence intensity are convenient features to characterize the concentration/amount of the NCs in solution and on surface. Based on these, the surface NCs assembled under different conditions were directly characterized with microscope imaging of the near IR photoluminescence from the NCs. The spin-coating method with multi-drops of sample applied during spinning process deposits more material on the ITO surface. Aligned bright spots were observed in the fluorescent images, which are the results of the fast evaporation of solvent DCM during the spinning. Similar features were observed throughout the ITO surface away from the center spot where the drops were added (not shown). The dip-coating method is less effective to deposit a large amount of NCs on the ITO electrode surface, particularly with less incubation time. With 5 minutes incubation in the dip-pull procedure, the overall surface coverage increased but the distribution is still not uniform. From both the ECL and photoluminescence imaging results, the spin-coating method deposited more NCs with better surface coverage on the ITO electrode surface.

The consistent aggregate spots evenly distributed on the ITO surface show that, by fine-tuning the solvent evaporation rate and spin speed, and with an appropriate affinity difference between the ITO surface and solvent with the NCs, more uniform surface distributions or films can be obtained. DCM has a low boiling point around 40° C. The fast evaporation rate of DCM makes slow surface preparations, which is generally more favorable to prepare more uniform surface films, technically difficult. Multiple drop addition, relative slow spin speed, and longer incubation time, were adopted to extend the sample-surface interaction time. Other solvents such as ACN (boiling point 82° C.) and chloroform (boiling point 60° C.) can be introduced. In addition to lower evaporation rates (i.e. slower evaporation), poorer solvent for the bimetallic NCs should also increase the NCs' affinity/interaction with ITO surface and self-assembly processes relative to DCM.

The bimetallic Au₁₂Ag₁₃ NCs are less soluble in pure acetonitrile (ACN) compared to other solvent such as DCM. The Au₁₂Ag₁₃ NCs stock solution in DCM (absorbance about 0.6 at 365 nm) was diluted with ACN to 1:1 volume ratio. Besides the slower evaporation rate of the solvent during spin coating, the changes in solvent polarity/affinity will also affect other interactions such as NCs with ITO surface and NCs themselves that affect the surface morphology or assembly pattern, and the correspondingly ECL and other properties.

Metal Nanoclusters Assembled on ITO Electrode Surface by Spin-Coating with Mixed Solvents

When spin-coated with mixed solvent DCM and ACN, the Au₁₂Ag₁₃ NCs self-assemble into microcrystals on the ITO surface. Such NC microcrystals, ordered assemblies on the surface, and their solid-state photoluminescence have not been previously observed. The results herein open a new paradigm for the production and use of atomically precise nanoclusters, both from the fundamental perspective and for applications based on their physiochemical properties. The dimension of individual microcrystals, the distribution and coverage of the microcrystals, as well as their assembly can be optimized. Those depend on parameters such as the solvent ratio, NC concentration, and spin speed and ITO electrode surface preparations etc.

The self-annihilation ECL is weak but detectable (data not shown).

Higher surface coverage and more uniform films were obtained by spin-coating with mixed DCM:chloroform at 1:1 volume ratio compared to DCM alone. The solubility of Au₁₂Ag₁₃ NCs in DCM and chloroform is similar. Therefore, the changes in solvent affinity/interactions with NCs or ITO surface should be insignificant. Fluorescence images of surface assembled NCs prepared by spin-coating with 1:1 DCM:chloroform as mixed solvent show no microcrystals. The slower solvent evaporation rate allows better NCs-ITO interactions which produces better surface distribution and coverage. Accumulated dry spots under slow spin speed were observed (data not shown). By adopting faster spin speed and less drop volume (900 RPM & 3 μL), a highly uniform film across a large coverage area around 0.5 cm² was deposited on the ITO electrode. The whole view of ITO shows only few brighter spots. The whole area is emissive and thus no contrast is available within the fluorescence image with a few brighter spots corresponding to the few aggregates of the NCs over the highly uniform emissive film (not dark background). ECL from this NC film display similar qualitative features compared to other surface assembled NCs.

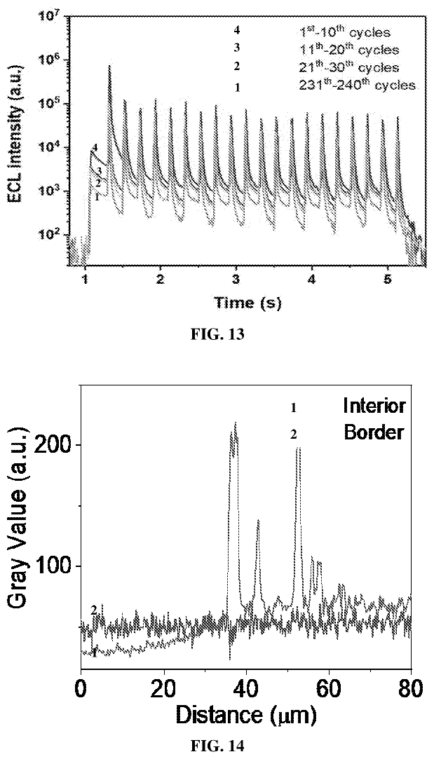

To better illustrate the uniformness of the NC film prepared with the introduction of chloroform, more quantitative image analysis was performed. The edge of the ITO electrode and the edge of the NCs surface film were included as comparison. The top line-profile in FIG. 14 at the distance <20 μm is an area outside of the NC film. A low photon counts- and less “noisy” region, statistically significant, serves as the background contrast. The high intensity peaks in the top line profile are the bright spots near the edge of the NC film corresponding to evaporation induced aggregates. The intensity of the interior film (i.e., the NC film) is consistent (at around 50 a.u. herein). The “noise” level is also higher than the blank ITO where no emissive NCs are available. This does not indicate inhomogeneity but rather a result from the optical diffraction limit and the hardware (camera/optics) resolution at hundreds of nanometers which are much larger than the NCs (about ≤2 nm in core size). The analysis here can be used to quantitate films prepared under systematically varied conditions and correlate with ECL and other function studies.

Surface Near-IR ECL Enhancement by Tertiary Amine Containing Drug

The near-IR ECL of Au₁₂Ag₁₃ NCs generated through solution diffusion processes, via self-annihilation and coreactant pathways, is described in Examples 1-5. With coreactant TPrA, the ECL signal was 400-fold stronger than that of Ru(bpy)₃ standard under the same conditions. The ECL from the integrated Au₁₂Ag₁₃ NCs-ITO electrode is measured in the absence or presence of piperazine drug which contains appropriate tertiary amine to enhance the near IR ECL signal.

The enhancement in near-IR ECL signal was demonstrated with tertiary amine containing drug cetirizine, which is the analyte of interest and also acts as a coreactant to increase the ECL signal. The compound cetirizine dihydrochloride, commonly referred to as cetirizine hydrochloride or cetirizine HCl, is the active ingredients of commercial drug Zyrtec. Zyrtec was approved by the FDA (U.S. Food and Drug Administration) as prescription in 1995, then approved for OTC (over-the-counter) drug in 2007 (FDA, Clinical pharmacology and biopharmaceutics review of cetirizine Research, C.F.D.E.A., Ed. 2010). It is a non-drowsy antihistamine that temporary reliefs the symptoms like itching swelling eyes, runny nose and sneezing from respiratory allergies or hay fever, also reduces uncomplicated skin pruritus from insect bites. Zyrtec contains 10 mg of cetirizine HCl in each tablet; the molecular formula is C₂₁H₂₅ClN₂O₃.2HCl with molecular weight 461.82 g/mol, the chemical structure is provided below. One of the tertiary amine groups has a pKa around 8, the other amine group closer to the aromatic groups has a pKa of 2.2, and the carboxyl group has a pKa of 2.9 (FDA, Clinical pharmacology and biopharmaceutics review of cetirizine Research, C.F.D.E.A., Ed. 2010; Hasan, et al., Int J Nanomedicine, 7:3351-3363 (2012); Testa, et al., Clinical & Experimental Allergy, 27(s2):13-18 (1997)). At physiological pH 7.4, the amine group with pKa 2.2 is fully deprotonated and the other one is partially deprotonated. Cetirizine was used as prototype for future tertiary amine drug sensing development due to its easy accessibility and low cost.

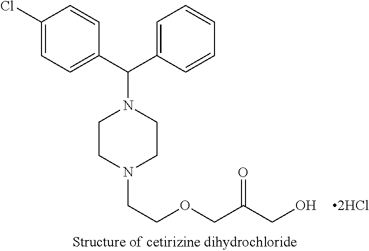 [SMILES: O=C(CO)COCCN1CCN(C(c2ccccc2)c2ccc(Cl)cc2)CC1]

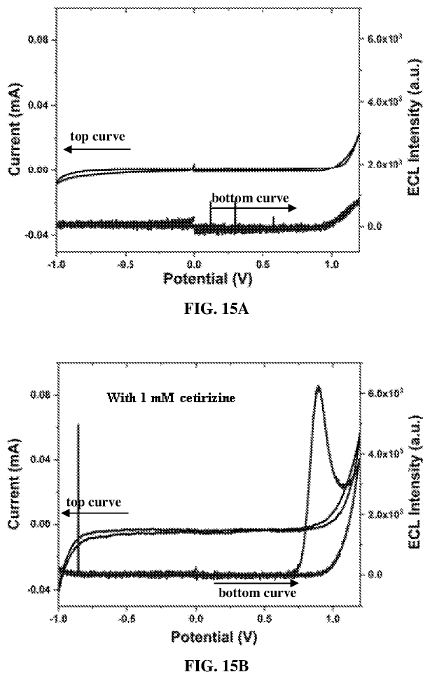

Cetirizine is the analyte of interest which can also act as a coreactant that enhances the ECL signal of surface assembled NCs prepared with DCM. A distinct ECL signal is detected at slightly less than +1.0 V (updated with the peak potential) with cetirizine added in the measurement solution shown in FIGS. 15A and 15B. The results demonstrate the signal-on response to the cetirizine drug analyte and confirm the efficiency of its function as ECL coreactants. The anodic current also increase in the same potential range. A defined current peak was not resolved (limited by possible water/solvent oxidation that would result in much higher background current). Therefore, ECL is a more suitable signal for detection of cetirizine over current in the system of Au₁₂Ag₁₃ NCs immobilized on ITO electrodes. The self-annihilation ECL was not observed in CV-ECL, unlike the near-IR ECL results in FIGS. 12 and 13 generated by potential steps. The difference is explained by the much longer time to sweep the potential within the chosen range of −1 V to 1.2 V in CV, during which the oxidized/reduced intermediate radicals would have undergone various possible side reactions.

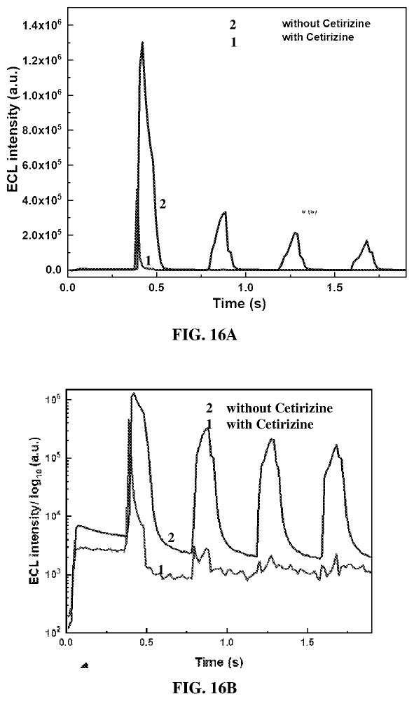

The impacts of cetirizine on the near IR ECL signal are directly compared under comparable conditions using potential step methods in FIGS. 16A and 16B. Both peak intensity and duration (peak area, or total ECL intensity) are drastically enhanced in the presence of cetirizine. Protonation of tertiary amine into quart ammonium ion is known to inhibit the ECL enhancement. At physiological pH 7.4, most nitrogen atoms in cetirizine remain deprotonated (the pKas of the two amine groups are ca. pKa 2.2 for the one closer to the aromatic rings pKa 8 for the other). The combinations of potential step parameters, −1.0 V for 0.3 s and +1.0 V for 0.1 s, for surface ECL measurements can be further optimized. Generally, applying a negative potential first with longer holding time induces higher ECL signal when an oxidation potential is subsequently applied. A faster potential step/switching is expected to reduce possible side reactions but could also generate less radicals or photons for a given exposure time.

Semi-quantitative correlations of the ECL signal with the amount of NCs used in the surface deposition are attempted. Because ECL or electrochemical reaction in general are interfacial processes, it is not necessarily true that higher concentration materials will be better in signal generation. Indeed, decreasing the total amount of NCs deposited did not cause statistically different ECL signals. Better reproducibility can establish a more quantitative trend. Regardless, the ECL signals are distinguishably higher than the signal without cetirizine in the solution. More consistent surface deposition and optimized measurement parameters should establish the calibration profiles for cetirizine or other analytes.

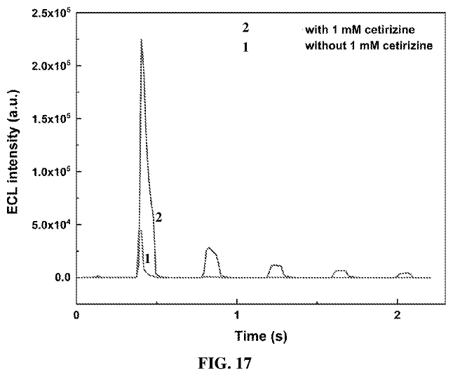

Cetirizine is also found to enhance the near-IR ECL signal of surface assembled NCs prepared with mixed solvents, i.e., DCM/chloroform. The enhanced ECL by 1 mM cetirizine is shown in FIG. 17. Qualitatively, the ECL features are similar to other surface deposited NCs with amorphous structures, displaying a peak by stepping to positive potentials followed by a gradual decay. Systematic measurements and characterization of the surface concentration or amount of NCs will allow more quantitative comparison.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. An electrochemiluminescence sensor comprising metal nanoclusters, wherein each of the metal nanoclusters comprises a metal core and a plurality of ligands, and wherein the plurality of ligands do not contain methionine.

2. The electrochemiluminescence sensor of claim 1, wherein the metal nanoclusters are organo-soluble or aqueous soluble.

3. (canceled)

4. The electrochemiluminescence sensor of claim 1, wherein the metal core comprises metal atoms of the same type or a mixture of metal atoms of different types.

5. The electrochemiluminescence sensor of claim 1, wherein the ligands comprise thiolates, phosphines, halogens, or combinations thereof.

6. The electrochemiluminescence sensor of claim 4, wherein the metal atoms are selected from the group consisting of gold, silver, aluminum, tin, magnesium, copper, nickel, iron, cobalt, magnesium, platinum, palladium, iridium, vanadium, rhodium, and ruthenium.

7. The electrochemiluminescence sensor of claim 4, wherein the metal atoms are gold.

8. The electrochemiluminescence sensor of claim 4, wherein the mixture of metal atoms contains gold and silver.

9. The electrochemiluminescence sensor of claim 1, wherein the metal nanoclusters further comprise targeting moieties bound to the core, to the ligands, or to both the core and the ligands of the metal nanoclusters.

10. The electrochemiluminescence sensor of claim 1 further comprising a conductive substrate.

11. The electrochemiluminescence sensor of claim 10, wherein the metal nanoclusters are assembled on the surface of the conductive substrate.

12. The electrochemiluminescence sensor of claim 1 further comprising a coreactant.

13. The electrochemiluminescence sensor of claim 12, wherein the coreactant is associated with the metal nanoclusters covalently or non-covalently.

14. The electrochemiluminescence sensor of claim 12, wherein the coreactant is selected from the group consisting of amines, oxalates, persulfates, hydrogen peroxide, nitrile, unsubstituted cyano, substituted cyano, unsubstituted benzophenone, substituted benzophenone, unsubstituted benzoic acid, substituted benzoic acid, unsubstituted naphthalene, substituted naphthalene, unsubstituted biphenyl, and substituted biphenyl.

15. The electrochemiluminescence sensor of claim 12, wherein the coreactant is an amine.

16. The electrochemiluminescence sensor of claim 12, wherein the coreactant is a tertiary amine.

17. The electrochemiluminescence sensor of claim 1, wherein the metal nanoclusters display near-IR electrochemiluminescence.

18. The electrochemiluminescence sensor of claim 1, wherein the metal nanoclusters display electrochemiluminescence higher than tris(bipyridine)ruthenium(II) complex under the same conditions.

19. The electrochemiluminescence sensor of claim 1, wherein the metal nanoclusters display electrochemiluminescence that is at least 2 times, at least 5 times, at least 10 times, at least 20 times, at least 25 times, at least 30 times, at least 50 times, at least 100 times, at least 150 times, at least 200 times, at least 250 times, at least 300 times, at least 350 times, or at least 400 times higher than tris(bipyridine)ruthenium(II) complex under the same conditions.

20. The electrochemiluminescence sensor of claim 1, wherein the metal nanoclusters are rod-shaped.

21. An electrochemiluminescence sensing array comprising two or more of the electrochemiluminescence sensors of claim 1.

22. A method of testing the presence, absence, or concentration of an analyte of interest in a sample, the method comprising:
(i) contacting the sample with the electrochemiluminescence sensor of claim 1,
(ii) applying a potential to the sensor, and
(iii) detecting the electrochemiluminescence and/or a redox current of the metal nanoclusters.

23. A method of screening the presence, absence, or concentration of a plurality of analytes of interest in a sample, the method comprising:
(i) contacting the sample with the electrochemiluminescence sensor array of claim 21,
(ii) applying a potential to the sensor, and
(iii) detecting the electrochemiluminescence and/or redox currents of the metal nanoclusters.

24. The method of claim 23, wherein the potential applied is the same or different for each of the electrochemiluminescence sensors.

25. The method of claim 22, wherein the potential is applied by linear sweeping from a first potential to a second potential, cyclic sweeping between a first potential and a second potential, or stepping between a first potential to a second potential.

26. The method of claim 22, wherein the potential is sufficient to provide enough energy to activate the corresponding energy states of the metal nanoclusters, the coreactant, the analyte, or combinations thereof.

27. The method of claim 22, wherein the analyte interacts with the metal nanoclusters and/or the coreactant.

28. The method of claim 22, wherein the electrochemiluminescence of the metal nanoclusters increases or decreases upon an interaction between the analyte and the metal nanoclusters and/or the coreactant as compared to the electrochemiluminescence of the metal nanoclusters in the absence of the analyte.

29. The method of claim 28, wherein the level of increase or decrease of the electrochemiluminescence of the metal nanoclusters is correlated to the concentration of the analyte.

30. The method of claim 22, wherein the redox current of the metal nanoclusters increases or decreases upon an interaction between the analyte and the metal nanoclusters and/or the coreactant as compared to the redox current of the metal nanoclusters in the absence of the analyte.

31. The method of claim 30, wherein the level of increase or decrease of the redox current of the metal nanoclusters is correlated to the concentration of the analyte.

32. The method of claim 22, wherein the sample is a buffer solution, a biological sample, or a combination of both.

33. The method of claim 22, wherein the sample is a biological sample, wherein the biological sample is a bodily fluid or mucus selected from the group consisting of saliva, sputum, tear, sweat, urine, exudate, blood, serum, plasma, and vaginal discharge.

34. The method of claim 22, wherein the analyte is a drug, metabolite, biomarker, metal ion, or combinations thereof.

35. The method of claim 22, wherein the analyte is a piperazine derivative drug.

36. The method of claim 22, wherein the electrochemiluminescence of the metal nanoclusters is detected by a camera.
